SEQUENCE LISTING (80 sequences)

SEQ ID NO: 1 - DNA, 2160 bp, Artificial Sequence
  Promoter
ctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcacc

SEQ ID NO: 2 - DNA, 774 bp, Artificial Sequence
  trpC terminator from Aspergillus nidulans
cgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcga

SEQ ID NO: 3 - DNA, 15739 bp, Artificial Sequence
  Vector
gatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggg

SEQ ID NO: 4 - DNA, 11611 bp, Artificial Sequence
  Vector
agcttgcatgcctgcaggtcgagtggagatgtggagtgggcgcttacacagtacacgaggacttctagctagaaagaaggattacctctaaacaagtgtacctgtgcattctgggtaaacgactcataggagagttgtaaaaaagtttcggccggcgtattgggtgttacggagcattcactaggcaaccatgcatggttactattgtataccatcttagtaggaantgatttcgaggtttatacctacgatgaatgtgtgtcctgtaggcttgagagttcaaggaagaaacatgcaattatctttgcgaacccaggngctggtgacggaattttcatagtcaagctatcagagtaaagaagaggagcatgtcaaagtacaattagagacaaatatatagtcgcgtggagccaagagcggattcctcagtctcgtaggtctcttgacgaccgttgatctgcttgatctcgtctcccgaaaatgaaaatagctctgctaagctattcttctcttcgccggagcctgnaaggcgttactaggttgcagtcaatgcattaatgcattgcagatgagctgtatctggaagaggtaaacccgaaaacgcgttttattcttgttgacatggagctattaaatcactagaaggcactctttgctgcttggacaaatgaacgtatcttatcgagatcctgaacaccatttgtctcaactccggagctgacatcgacaccaacgatcttatatccagattcgtcaagctgtttgatgatttcagtaacgttaagtggatcgatcccgcggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcatggtgatgtctgctcaagcggggtagctgttagtcaagctgcgatgaagtgggaaagctcgaactgaaaggttcaaaggaataagggatgggaaggatggagtatggatgtagcaaagtacttacttaggggaaataaaggttcttggatgggaagatgaatatactgaagatgggaaaagaaagagaaaagaaaagagcagctggtggggagagcaggaaaatatggcaacaaatgttggactgacgcaacgaccttgtcaaccccgccgacacaccgggcggacagacggggcaaagctgcctaccagggactgagggacctcagcaggtcgagtgcagagcaccggatgggtcgactgccagcttgtgttcccggtctgcgccgctggccagctcctgagcggcctttccggtttcatacaccgggcaaagcaggagaggcacgatatttggacgccctacagatgccggatgggccaattagggagcttacgcgccgggtactcgctctacctacttcggagaaggtactatctcgtgaatcttttaccagatcggaagcaattggacttctgtacctaggttaatggcatgctatttcgccgacggctatacacccctggcttcacattctccttcgcttactgccggtgattcgatgaagctccatattctccgatgatgcaatagattcttggtcaacgaggggcacaccagcctttccacttcggggcggaggggcggccggtcccggattaataatcatccactgcacctcagagccgccagagctgtctggccagtggcttattactcagcccttctctctgcgtccgtccgtctctccgcatgccagaaagagtcaccggtcactgtacagagctcacgagttcgtcacatttttctacaaatggtggaggcggcggattttaggctcaagtcatgaccctctgggtcactccagaatcagctaggtcaacgaataaggatgattctataggaagatccaggcaccggtcaaccatgatctggacagatttgggagctcggtataagctctccacctatcttattctgtatagtttaggcttaaagtttatccaggagatgttgctgaagtcgatttgagtccacttcctcactggtagctatacgactttgatggtcgttgtaggggctgtattaggtctcgatcaaacacaaatagaattaaatggtactcgagtccactgaaggtggcttctccgtcttccgtagccgtgccgaaatccttacagcttgtgttgtgtgacttttggttacgccgtctgacttttgtggtgagctaactagagatcatgctatatctcctgatttaatacaatgctcatcataacattccacctggaactgctagcaacgtttgacttgcattgtgcaacgccctttgcagagctatcggatgatcaatagtgccacgttctaaattcaaccaacgcaggtgccccaagccttcgacatccggatgtatttcgaaaacctcatggcgattgcagtcctcagattcatgttcattccaatgctcattggtgaataaaaggttcacagggaataagttcaaactcgagatacttgagaatattgaaagccaaaggaccctctatgctccaagctagagtctcagcctggaaagcaaatccaaatgaagctatgctacctccaattcctcatcatcttatctataatacagagtcgaagaatatcctcttgacaccgctccgtcctccgacttcaataaggagcttactcctccttgacaccacccctccagttcttctcggcgttctggagggaggccttgtcggtcttgggctggccctggctgagaaagctgttggcagccttaaagggacgctggaggtcaccagtcgctggcttcccgaagacgtggatcttaaccagattcgaaagcgccttcagcggatgatcgactggatcagaagagcgttggtgtacttgaagtacagatgcatgacggccatcatgccaacgcccatgaactggctcttaatgagctggcggaactggcccttatcgtactccatgttggtagttgtgacaggacgaggctcctcgccgcttccaagcggagcaggctcgacgtatttcagtgtcgaaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgcggccgcaattaaccctcactaaaggatccctatagtgagtcgtattatgcggccgcgaattctcatgtttgaccgcttatcatcgataagctctgctttttgttgacttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacaaggcggatcgccggaaaggacccgcaaatgataataattatcaattgcatactatcgacggcactgctgccagataacaccaccggggaaacattccatcatgatggccgtgcggacataggaagccagttcatccatcgctttcttgtctgctgccatttgctttgtgacatccagcgccgcacattcagcagcgtttttcagcgcgttttcgatcaacgtttcaatgttggtatcaacaccaggtttaactttgaacttatcggcactgacggttaccttgttctgcgctggctcatcacgcaggataccaaggctgatgttgtagatattggtcaccggctgagggttttcgattgccgctgcgtggatagcaccatttgcgatcaggcngtccttgatgaatgacactccattgcgaataagttcgaaggagacggtgtcacgaatgcgctggtccagctcggtcgattgccttttgtgcagcagaggtatcaatctcaacgccaaggctcatcgaagcgcaatattgctgctcaccaaaacgcgtattgaccaggtgttcaacggcaaatttctgcccttctgatgtcagaaaggcaaagtgattttctttctggtattcagttgctgtgtgtcggtttcagcaaaaccaagctcgcgcaattcggctgtgcagatttagaaggcagatcaccagacagcaacggccaacggaaaacagcgcatacagaacatccgtcgccgcgccgacaacgtgataatttttatgacccatgatttatttccttttagacgtgagcctgtcgcacagcaaagccgccgaaagttcctcgaagctagcttcagacgtgtctagatacgtctgctttttgttgacttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacaaggcggatcgccggaaaggacccgcaaatgataataattatcaattgcatactatcgacggcactgctgccagataacaccaccggggaaacattccatcatgatggccgtgcggacataggaagccagttcatccatcgctttcttgtctgctgccatttgctttgtgacatccagcgccgcacattcagcagcgtttttcagcgcgttttcgatcaacgtttcaatgttggtatcaacaccaggtttaactttgaacttatcggcactgacggttaccttgttctgcgctggctcatcacgcaggataccaaggctgatgttgtagatattggtcaccggctgagggttttcgattgccgctgcgtggatagcaccatttgcgatcaggcngtccttgatgaatgacactccattgcgaataagttcgaaggagacggtgtcacgaatgcgctggtccagctcggtcgattgccttttgtgcagcagaggtatcaatctcaacgccaaggctcatcgaagcgcaatattgctgctcaccaaaacgcgtattgaccaggtgttcaacggcaaatttctgcccttctgatgtcagaaaggcaaagtgattttctttctggtattcagttgctgtgtgtcggtttcagcaaaaccaagctcgcgcaattcggctgtgcagatttagaaggcagatcaccagacagcaacggccaacggaaaacagcgcatacagaacatccgtcgccgcgccgacaacgtgataatttttatgacccatgatttatttccttttagacgtgagcctgtcgcacagcaaagccgccgaaagttcctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctgcagatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaa

SEQ ID NO: 5 - DNA, 21 bp, Artificial Sequence
  Primer
cgatgtaggagggcgtggata

SEQ ID NO: 6 - DNA, 21 bp, Artificial Sequence
  Primer
gcttctgcgggcgatttgtgt

SEQ ID NO: 7 - DNA, 20 bp, Artificial Sequence
  Primer
tgagaatatcaccggaattg

SEQ ID NO: 8 - DNA, 21 bp, Artificial Sequence
  Primer
agctcgacatactgttcttcc

SEQ ID NO: 9 - DNA, 24 bp, Artificial Sequence
  Primer
gtgaatggaaatcccatcgctgtc

SEQ ID NO: 10 - DNA, 24 bp, Artificial Sequence
  Primer
agtgggtactctaaaggccatacc

SEQ ID NO: 11 - DNA, 1771 bp, Haematococcus pluvialis
  CDS (166)..(1155)
ggcacgagcttgcacgcaagtcagcgcgcgcaagtcaacacctgccggtccacagcctcaaataataaagagctcaagcgtttgtgcgcctcgacgtggccagtctgcactgccttgaacccgcgagtctcccgccgcactgactgccatagcacagctagacgaatgcagctagcagcgacagtaatgttggagcagcttaccggaagcgctgaggcactcaaggagaaggagaaggaggttgcaggcagctctgacgtgttgcgtacatgggcgacccagtactcgcttccgtcagaagagtcagacgcggcccgcccgggactgaagaatgcctacaagccaccaccttccgacacaaagggcatcacaatggcgctacgtgtcatcggctcctgggccgcagtgttcctccacgccatttttcaaatcaagcttccgacctccttggaccagctgcactggctgcccgtgtcagatgccacagctcagctggttagcggcacgagcagcctgctcgacatcgtcgtagtattctttgtcctggagttcctgtacacaggcctttttatcaccacgcatgatgctatgcatggcaccatcgccatgagaaacaggcagcttaatgacttcttgggcagagtatgcatctccttgtacgcctggtttgattacaacatgctgcaccgcaagcattgggagcaccacaaccacactggcgaggtgggcaaggaccctgacttccacaggggaaaccctggcattgtgccctggtttgccagcttcatgtccagctacatgtcgatgtggcagtttgcgcgcctcgcatggtggacggtggtcatgcagctgctgggtgcgccaatggcgaacctgctggtgttcatggcggccgcgcccatcctgtccgccttccgcttgttctactttggcacgtacatgccccacaagcctgagcctggcgccgcgtcaggctcttcaccagccgtcatgaactggtggaagtcgcgcactagccaggcgtccgacctggtcagctttctgacctgctaccacttcgacctgcactgggagcaccaccgctggcccttcgccccctggtgggagctgcccaactgccgccgcctgtctggccgaggtctggttcctgcctagctggacacactgcagtgggccctgctgccagctgggcatgcaggttgtggcaggactgggtgaggtgaaaagctgcaggcgctgctgccggacacgctgcatgggctaccctgtgtagctgccgccactaggggagggggtttgtagctgtcgagcttgccccatggatgaagctgtgtagtggtgcagggagtacacccacaggccaacacccttgcaggagatgtcttgcgtcgggaggagtgttgggcagtgtagatgctatgattgtatcttaatgctgaagcctttaggggagcgacacttagtgctgggcaggcaacgccctgcaaggtgcaggcacaagctaggctggacgaggactcggtggcaggcaggtgaagaggtgcgggagggtggtgccacacccactgggcaagaccatgctgcaatgctggcggtgtggcagtgagagctgcgtgattaactgggctatggattgtttgagcagtctcacttattctttgatatagatactggtcaggcaggtcaggagagtgagtatgaacaagttgagaggtggtgcgctgcccctgcgcttatgaagctgtaacaataaagtggttcaaaaaaaaaa

SEQ ID NO: 12 - protein, 329 aa, Haematococcus pluvialis
MQLAATVMLEQLTGSAEALKEKEKEVAGSSDVLRTWATQYSLPSEESDAARPGLKNAYKPPPSDTKGITMALRVIGSWAAVFLHAIFQIKLPTSLDQLHWLPVSDATAQLVSGTSSLLDIVVVFFVLEFLYTGLFITTHDAMHGTIAMRNRQLNDFLGRVCISLYAWFDYNMLHRKHWEHHNHTGEVGKDPDFHRGNPGIVPWFASFMSSYMSMWQFARLAWWTVVMQLLGAPMANLLVFMAAAPILSAFRLFYFGTYMPHKPEPGAASGSSPAVMNWWKSRTSQASDLVSFLTCYHFDLHWEHHRWPFAPWWELPNCRRLSGRGLVPA

SEQ ID NO: 13 - DNA, 1662 bp, Haematococcus pluvialis
  CDS (168)..(1130)
cggggcaactcaagaaattcaacagctgcaagcgcgccccagcctcacagcgccaagtgagctatcgacgtggttgtgagcgctcgacgtggtccactgacgggcctgtgagcctctgcgctccgtcctctgccaaatctcgcgtcggggcctgcctaagtcgaagaatgcacgtcgcatcggcactaatggtcgagcagaaaggcagtgaggcagctgcttccagcccagacgtcttgagagcgtgggcgacacagtatcacatgccatccgagtcgtcagacgcagctcgtcctgcgctaaagcacgcctacaaacctccagcatctgacgccaagggcatcacgatggcgctgaccatcattggcacctggaccgcagtgtttttacacgcaatatttcaaatcaggctaccgacatccatggaccagcttcactggttgcctgtgtccgaagccacagcccagcttttgggcggaagcagcagcctactgcacatcgctgcagtcttcattgtacttgagttcctgtacactggtctattcatcaccacacatgacgcaatgcatggcaccatagctttgaggcacaggcagctcaatgatctccttggcaacatctgcatatcactgtacgcctggtttgactacagcatgctgcatcgcaagcactgggagcaccacaaccatactggcgaagtggggaaagaccctgacttccacaagggaaatcccggccttgtcccctggttcgccagcttcatgtccagctacatgtccctgtggcagtttgcccggctggcatggtgggcagtggtgatgcaaatgctgggggcgcccatggcaaatctcctagtcttcatggctgcagccccaatcttgtcagcattccgcctcttctacttcggcacttacctgccacacaagcctgagccaggccctgcagcaggctctcaggtgatggcctggttcagggccaagacaagtgaggcatctgatgtgatgagtttcctgacatgctaccactttgacctgcactgggagcaccacaggtggccctttgccccctggtggcagctgccccactgccgccgcctgtccgggcgtggcctggtgcctgccttggcatgacctggtccctccgctggtgacccagcgtctgcacaagagtgtcatgctacagggtgctgcggccagtggcagcgcagtgcactctcagcctgtatggggctaccgctgtgccactgagcactgggcatgccactgagcactgggcgtgctactgagcaatgggcgtgctactgagcaatgggcgtgctactgacaatgggcgtgctactggggtctggcagtggctaggatggagtttgatgcattcagtagcggtggccaacgtcatgtggatggtggaagtgctgaggggtttaggcagccggcatttgagagggctaagttataaatcgcatgctgctcatgcgcacatatctgcacacagccagggaaatcccttcgagagtgattatgggacacttgtattggtttcgtgctattgttttattcagcagcagtacttagtgagggtgagagcagggtggtgagagtggagtgagtgagtatgaacctggtcagcgaggtgaacagcctgtaatgaatgactctgtct

SEQ ID NO: 14 - protein, 320 aa, Haematococcus pluvialis
MHVASALMVEQKGSEAAASSPDVLRAWATQYHMPSESSDAARPALKHAYKPPASDAKGITMALTIIGTWTAVFLHAIFQIRLPTSMDQLHWLPVSEATAQLLGGSSSLLHIAAVFIVLEFLYTGLFITTHDAMHGTIALRHRQLNDLLGNICISLYAWFDYSMLHRKHWEHHNHTGEVGKDPDFHKGNPGLVPWFASFMSSYMSLWQFARLAWWAVVMQMLGAPMANLLVFMAAAPILSAFRLFYFGTYLPHKPEPGPAAGSQVMAWFRAKTSEASDVMSFLTCYHFDLHWEHHRWPFAPWWQLPHCRRLSGRGLVPALA

SEQ ID NO: 15 - DNA, 729 bp, Agrobacterium aurantiacum
  CDS (1)..(729)
atgagcgcacatgccctgcccaaggcagatctgaccgccaccagcctgatcgtctcgggcggcatcatcgccgcttggctggccctgcatgtgcatgcgctgtggtttctggacgcagcggcgcatcccatcctggcgatcgcaaatttcctggggctgacctggctgtcggtcggattgttcatcatcgcgcatgacgcgatgcacgggtcggtggtgccggggcgtccgcgcgccaatgcggcgatgggccagcttgtcctgtggctgtatgccggattttcgtggcgcaagatgatcgtcaagcacatggcccatcaccgccatgccggaaccgacgacgaccccgatttcgaccatggcggcccggtccgctggtacgcccgcttcatcggcacctatttcggctggcgcgaggggctgctgctgcccgtcatcgtgacggtctatgcgctgatccttggggatcgctggatgtacgtggtcttctggccgctgccgtcgatcctggcgtcgatccagctgttcgtgttcggcacctggctgccgcaccgccccggccacgacgcgttcccggaccgccacaatgcgcggtcgtcgcggatcagcgaccccgtgtcgctgctgacctgctttcactttggcggttatcatcacgaacaccacctgcacccgacggtgccgtggtggcgcctgcccagcacccgcaccaagggggacaccgcatga

SEQ ID NO: 16 - protein, 242 aa, Agrobacterium aurantiacum
MSAHALPKADLTATSLIVSGGIIAAWLALHVHALWFLDAAAHPILAIANFLGLTWLSVGLFIIAHDAMHGSVVPGRPRANAAMGQLVLWLYAGFSWRKMIVKHMAHHRHAGTDDDPDFDHGGPVRWYARFIGTYFGWREGLLLPVIVTVYALILGDRWMYVVFWPLPSILASIQLFVFGTWLPHRPGHDAFPDRHNARSSRISDPVSLLTCFHFGGYHHEHHLHPTVPWWRLPSTRTKGDTA

SEQ ID NO: 17 - DNA, 1631 bp, Alcaligenes sp.
  CDS (99)..(827)
ctgcaggccgggcccggtggccaatggtcgcaaccggcaggactggaacaggacggcgggccggtctaggctgtcgccctacgcagcaggagtttcggatgtccggacggaagcctggcacaactggcgacacgatcgtcaatctcggtctgaccgccgcgatcctgctgtgctggctggtcctgcacgcctttacgctatggttgctagatgcggccgcgcatccgctgcttgccgtgctgtgcctggctgggctgacctggctgtcggtcgggctgttcatcatcgcgcatgacgcaatgcacgggtccgtggtgccggggcggccgcgcgccaatgcggcgatcgggcaactggcgctgtggctctatgcggggttctcgtggcccaagctgatcgccaagcacatgacgcatcaccggcacgccggcaccgacaacgatcccgatttcggtcacggagggcccgtgcgctggtacggcagcttcgtctccacctatttcggctggcgagagggactgctgctaccggtgatcgtcaccacctatgcgctgatcctgggcgatcgctggatgtatgtcatcttctggccggtcccggccgttctggcgtcgatccagattttcgtcttcggaacttggctgccccaccgcccgggacatgacgattttcccgaccggcacaacgcgaggtcgaccggcatcggcgacccgttgtcactactgacctgcttccatttcggcggctatcaccacgaacatcacctgcatccgcatgtgccgtggtggcgcctgcctcgtacacgcaagaccggaggccgcgcatgacgcaattcctcattgtcgtggcgacagtcctcgtgatggagctgaccgcctattccgtccaccgctggattatgcacggccccctaggctggggctggcacaagtcccatcacgaagagcacgaccacgcgttggagaagaacgacctctacggcgtcgtcttcgcggtgctggcgacgatcctcttcaccgtgggcgcctattggtggccggtgctgtggtggatcgccctgggcatgacggtctatgggttgatctatttcatcctgcacgacgggcttgtgcatcaacgctggccgtttcggtatattccgcggcggggctatttccgcaggctctaccaagctcatcgcctgcaccacgcggtcgaggggcgggaccactgcgtcagcttcggcttcatctatgccccacccgtggacaagctgaagcaggatctgaagcggtcgggtgtcctgcgcccccaggacgagcgtccgtcgtgatctctgatcccggcgtggccgcatgaaatccgacgtgctgctggcaggggccggccttgccaacggactgatcgcgctggcgatccgcaaggcgcggcccgaccttcgcgtgctgctgctggaccgtgcggcgggcgcctcggacgggcatacttggtcctgccacgacaccgatttggcgccgcactggctggaccgcctgaagccgatcaggcgtggcgactggcccgatcaggaggtgcggttcccagaccattcgcgaaggctccgggccggatatggctcgatcgacgggcgggggctgatgcgtgcggtgacc

SEQ ID NO: 18 - protein, 242 aa, Alcaligenes sp.
MSGRKPGTTGDTIVNLGLTAAILLCWLVLHAFTLWLLDAAAHPLLAVLCLAGLTWLSVGLFIIAHDAMHGSVVPGRPRANAAIGQLALWLYAGFSWPKLIAKHMTHHRHAGTDNDPDFGHGGPVRWYGSFVSTYFGWREGLLLPVIVTTYALILGDRWMYVIFWPVPAVLASIQIFVFGTWLPHRPGHDDFPDRHNARSTGIGDPLSLLTCFHFGGYHHEHHLHPHVPWWRLPRTRKTGGRA

SEQ ID NO: 19 - DNA, 729 bp, Paracoccus marcusii
  CDS (1)..(729)
atgagcgcacatgccctgcccaaggcagatctgaccgccacaagcctgatcgtctcgggcggcatcatcgccgcatggctggccctgcatgtgcatgcgctgtggtttctggacgcggcggcccatcccatcctggcggtcgcgaatttcctggggctgacctggctgtcggtcggattgttcatcatcgcgcatgacgcgatgcacgggtcggtcgtgccggggcgtccgcgcgccaatgcggcgatgggccagcttgtcctgtggctgtatgccggattttcgtggcgcaagatgatcgtcaagcacatggcccatcaccgccatgccggaaccgacgacgacccagatttcgaccatggcggcccggtccgctggtacgcccgcttcatcggcacctatttcggctggcgcgaggggctgctgctgcccgtcatcgtgacggtctatgcgctgatcctgggggatcgctggatgtacgtggtcttctggccgttgccgtcgatcctggcgtcgatccagctgttcgtgttcggcacttggctgccgcaccgccccggccacgacgcgttcccggaccgccataatgcgcggtcgtcgcggatcagcgaccctgtgtcgctgctgacctgctttcattttggcggttatcatcacgaacaccacctgcacccgacggtgccgtggtggcgcctgcccagcacccgcaccaagggggacaccgcatga

SEQ ID NO: 20 - protein, 242 aa, Paracoccus marcusii
MSAHALPKADLTATSLIVSGGIIAAWLALHVHALWFLDAAAHPILAVANFLGLTWLSVGLFIIAHDAMHGSVVPGRPRANAAMGQLVLWLYAGFSWRKMIVKHMAHHRHAGTDDDPDFDHGGPVRWYARFIGTYFGWREGLLLPVIVTVYALILGDRWMYVVFWPLPSILASIQLFVFGTWLPHRPGHDAFPDRHNARSSRISDPVSLLTCFHFGGYHHEHHLHPTVPWWRLPSTRTKGDTA

SEQ ID NO: 21 - DNA, 1629 bp, Synechocystis sp.
  CDS (1)..(1629)
atgatcaccaccgatgttgtcattattggggcggggcacaatggcttagtctgtgcagcctatttgctccaacggggcttgggggtgacgttactagaaaagcgggaagtaccagggggggcggccaccacagaagctctcatgccggagctatccccccagtttcgctttaaccgctgtgccattgaccacgaatttatctttctggggccggtgttgcaggagctaaatttagcccagtatggtttggaatatttattttgtgaccccagtgttttttgtccggggctggatggccaagcttttatgagctaccgttccctagaaaaaacctgtgcccacattgccacctatagcccccgagatgcggaaaaatatcggcaatttgtcaattattggacggatttgctcaacgctgtccagcctgcttttaatgctccgccccaggctttactagatttagccctgaactatggttgggaaaacttaaaatccgtgctggcgatcgccgggtcgaaaaccaaggcgttggattttatccgcactatgatcggctccccggaagatgtgctcaatgaatggttcgacagcgaacgggttaaagctcctttagctagactatgttcggaaattggcgctcccccatcccaaaagggtagtagctccggcatgatgatggtggccatgcggcatttggagggaattgccagaccaaaaggaggcactggagccctcacagaagccttggtgaagttagtgcaagcccaagggggaaaaatcctcactgaccaaaccgtcaaacgggtattggtggaaaacaaccaggcgatcggggtggaggtagctaacggagaacagtaccgggccaaaaaaggcgtgatttctaacatcgatgcccgccgtttatttttgcaattggtggaaccgggggccctagccaaggtgaatcaaaacctaggggaacgactggaacggcgcactgtgaacaataacgaagccattttaaaaatcgattgtgccctctccggtttaccccacttcactgccatggccgggccggaggatctaacgggaactattttgattgccgactcggtacgccatgtcgaggaagcccacgccctcattgccttggggcaaattcccgatgctaatccgtctttatatttggatattcccactgtattggaccccaccatggccccccctgggcagcacaccctctggatcgaattttttgccccctaccgcatcgccgggttggaagggacagggttaatgggcacaggttggaccgatgagttaaaggaaaaagtggcggatcgggtgattgataaattaacggactatgcccctaacctaaaatctctgatcattggtcgccgagtggaaagtcccgccgaactggcccaacggctgggaagttacaacggcaatgtctatcatctggatatgagtttggaccaaatgatgttcctccggcctctaccggaaattgccaactaccaaacccccatcaaaaatctttacttaacaggggcgggtacccatcccggtggctccatatcaggtatgcccggtagaaattgcgctcgggtctttttaaaacaacaacgtcgtttttggtaa

SEQ ID NO: 22 - protein, 542 aa, Synechocystis sp.
MITTDVVIIGAGHNGLVCAAYLLQRGLGVTLLEKREVPGGAATTEALMPELSPQFRFNRCAIDHEFIFLGPVLQELNLAQYGLEYLFCDPSVFCPGLDGQAFMSYRSLEKTCAHIATYSPRDAEKYRQFVNYWTDLLNAVQPAFNAPPQALLDLALNYGWENLKSVLAIAGSKTKALDFIRTMIGSPEDVLNEWFDSERVKAPLARLCSEIGAPPSQKGSSSGMMMVAMRHLEGIARPKGGTGALTEALVKLVQAQGGKILTDQTVKRVLVENNQAIGVEVANGEQYRAKKGVISNIDARRLFLQLVEPGALAKVNQNLGERLERRTVNNNEAILKIDCALSGLPHFTAMAGPEDLTGTILIADSVRHVEEAHALIALGQIPDANPSLYLDIPTVLDPTMAPPGQHTLWIEFFAPYRIAGLEGTGLMGTGWTDELKEKVADRVIDKLTDYAPNLKSLIIGRRVESPAELAQRLGSYNGNVYHLDMSLDQMMFLRPLPEIANYQTPIKNLYLTGAGTHPGGSISGMPGRNCARVFLKQQRRFW

SEQ ID NO: 23 - DNA, 776 bp, Bradyrhizobium sp.
  CDS (1)..(774)
atgcatgcagcaaccgccaaggctactgagttcggggcctctcggcgcgacgatgcgaggcagcgccgcgtcggtctcacgctggccgcggtcatcatcgccgcctggctggtgctgcatgtcggtctgatgttcttctggccgctgacccttcacagcctgctgccggctttgcctctggtggtgctgcagacctggctctatgtaggcctgttcatcatcgcgcatgactgcatgcacggctcgctggtgccgttcaagccgcaggtcaaccgccgtatcggacagctctgcctgttcctctatgccgggttctccttcgacgctctcaatgtcgagcaccacaagcatcaccgccatcccggcacggccgaggatcccgatttcgacgaggtgccgccgcacggcttctggcactggttcgccagctttttcctgcactatttcggctggaagcaggtcgcgatcatcgcagccgtctcgctggtttatcagctcgtcttcgccgttcccttgcagaacatcctgctgttctgggcgctgcccgggctgctgtcggcgctgcagctgttcaccttcggcacctatctgccgcacaagccggccacgcagcccttcgccgatcgccacaacgcgcggacgagcgaatttcccgcgtggctgtcgctgctgacctgcttccacttcggctttcatcacgagcatcatctgcatcccgatgcgccgtggtggcggctgccggagatcaagcggcgggccctggaaaggcgtgacta

SEQ ID NO: 24 - protein, 258 aa, Bradyrhizobium sp.
MHAATAKATEFGASRRDDARQRRVGLTLAAVIIAAWLVLHVGLMFFWPLTLHSLLPALPLVVLQTWLYVGLFIIAHDCMHGSLVPFKPQVNRRIGQLCLFLYAGFSFDALNVEHHKHHRHPGTAEDPDFDEVPPHGFWHWFASFFLHYFGWKQVAIIAAVSLVYQLVFAVPLQNILLFWALPGLLSALQLFTFGTYLPHKPATQPFADRHNARTSEFPAWLSLLTCFHFGFHHEHHLHPDAPWWRLPEIKRRALERRD

SEQ ID NO: 25 - DNA, 777 bp, Nostoc sp.
  CDS (1)..(777)
atggttcagtgtcaaccatcatctctgcattcagaaaaactggtgttattgtcatcgacaatcagagatgataaaaatattaataagggtatatttattgcctgctttatcttatttttatgggcaattagtttaatcttattactctcaatagatacatccataattcataagagcttattaggtatagccatgctttggcagaccttcttatatacaggtttatttattactgctcatgatgccatgcacggcgtagtttatcccaaaaatcccagaataaataattttataggtaagctcactctaatcttgtatggactactcccttataaagatttattgaaaaaacattggttacaccacggacatcctggtactgatttagaccctgattattacaatggtcatccccaaaacttctttctttggtatctacattttatgaagtcttattggcgatggacgcaaattttcggattagtgatgatttttcatggacttaaaaatctggtgcatataccagaaaataatttaattatattttggatgataccttctattttaagttcagtacaactattttattttggtacatttttgcctcataaaaagctagaaggtggttatactaacccccattgtgcgcgcagtatcccattacctcttttttggtcttttgttacttgttatcacttcggctaccacaaggaacatcacgaataccctcaacttccttggtggaaattacctgaagctcacaaaatatctttataa

SEQ ID NO: 26 - protein, 258 aa, Nostoc sp.
MVQCQPSSLHSEKLVLLSSTIRDDKNINKGIFIACFILFLWAISLILLLSIDTSIIHKSLLGIAMLWQTFLYTGLFITAHDAMHGVVYPKNPRINNFIGKLTLILYGLLPYKDLLKKHWLHHGHPGTDLDPDYYNGHPQNFFLWYLHFMKSYWRWTQIFGLVMIFHGLKNLVHIPENNLIIFWMIPSILSSVQLFYFGTFLPHKKLEGGYTNPHCARSIPLPLFWSFVTCYHFGYHKEHHEYPQLPWWKLPEAHKISL

SEQ ID NO: 27 - DNA, 789 bp, Nostoc punctiforme
  CDS (1)..(789)
ttgaatttttgtgataaaccagttagctattatgttgcaatagagcaattaagtgctaaagaagatactgtttgggggctggtgattgtcatagtaattattagtctttgggtagctagtttggcttttttactagctattaattatgccaaagtcccaatttggttgatacctattgcaatagtttggcaaatgttcctttatacagggctatttattactgcacatgatgctatgcatgggtcagtttatcgtaaaaatcccaaaattaataattttatcggttcactagctgtagcgctttacgctgtgtttccatatcaacagatgttaaagaatcattgcttacatcatcgtcatcctgctagcgaagttgacccagattttcatgatggtaagagaacaaacgctattttctggtatctccatttcatgatagaatactccagttggcaacagttaatagtactaactatcctatttaatttagctaaatacgttttgcacatccatcaaataaatctcatcttattttggagtattcctccaattttaagttccattcaactgttttatttcggaacatttttgcctcatcgagaacccaagaaaggatatgtttatccccattgcagccaaacaataaaattgccaacttttttgtcatttatcgcttgctaccactttggttatcatgaagaacatcatgagtatccccatgtaccttggtggcaacttccatctgtatataagcagagagtattcaacaattcagtaaccaattcgtaa

SEQ ID NO: 28 - protein, 262 aa, Nostoc punctiforme
LNFCDKPVSYYVAIEQLSAKEDTVWGLVIVIVIISLWVASLAFLLAINYAKVPIWLIPIAIVWQMFLYTGLFITAHDAMHGSVYRKNPKINNFIGSLAVALYAVFPYQQMLKNHCLHHRHPASEVDPDFHDGKRTNAIFWYLHFMIEYSSWQQLIVLTILFNLAKYVLHIHQINLILFWSIPPILSSIQLFYFGTFLPHREPKKGYVYPHCSQTIKLPTFLSFIACYHFGYHEEHHEYPHVPWWQLPSVYKQRVFNNSVTNS

SEQ ID NO: 29 - DNA, 762 bp, Nostoc punctiforme
  CDS (1)..(762)
gtgatccagttagaacaaccactcagtcatcaagcaaaactgactccagtactgagaagtaaatctcagtttaaggggcttttcattgctattgtcattgttagcgcatgggtcattagcctgagtttattactttcccttgacatctcaaagctaaaattttggatgttattgcctgttatactatggcaaacatttttatatacgggattatttattacatctcatgatgccatgcatggcgtagtatttccccaaaacaccaagattaatcatttgattggaacattgaccctatccctttatggtcttttaccatatcaaaaactattgaaaaaacattggttacaccaccacaatccagcaagctcaatagacccggattttcacaatggtaaacaccaaagtttctttgcttggtattttcattttatgaaaggttactggagttgggggcaaataattgcgttgactattatttataactttgctaaatacatactccatatcccaagtgataatctaacttacttttgggtgctaccctcgcttttaagttcattacaattattctattttggtacttttttaccccatagtgaaccaatagggggttatgttcagcctcattgtgcccaaacaattagccgtcctatttggtggtcatttatcacgtgctatcattttggctaccacgaggaacatcacgaatatcctcatatttcttggtggcagttaccagaaatttacaaagcaaaatag

SEQ ID NO: 30 - protein, 253 aa, Nostoc punctiforme
VIQLEQPLSHQAKLTPVLRSKSQFKGLFIAIVIVSAWVISLSLLLSLDISKLKFWMLLPVILWQTFLYTGLFITSHDAMHGVVFPQNTKINHLIGTLTLSLYGLLPYQKLLKKHWLHHHNPASSIDPDFHNGKHQSFFAWYFHFMKGYWSWGQIIALTIIYNFAKYILHIPSDNLTYFWVLPSLLSSLQLFYFGTFLPHSEPIGGYVQPHCAQTISRPIWWSFITCYHFGYHEEHHEYPHISWWQLPEIYKAK

SEQ ID NO: 31 - DNA, 1608 bp, Haematococcus pluvialis
  CDS (3)..(971)
ctacatttcacaagcccgtgagcggtgcaagcgctctgccccacatcggcccacctcctcatctccatcggtcatttgctgctaccacgatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagggtgcggaaccaggcacgctggtttcacacctcatgcctgtgataaggtgtggctagagcgatgcgtgtgagacgggtatgtcacggtcgactggtctgatggccaatggcatcggccatgtctggtcatcacgggctggttgcctgggtgaaggtgatgcacatcatcatgtgcggttggaggggctggcacagtgtgggctgaactggagcagttgtccaggctggcgttgaatcagtgagggtttgtgattggcggttgtgaagcaatgactccgcccatattctatttgtgggagctgagatgatggcatgcttgggatgtgcatggatcatggtagtgcagcaaactatattcacctagggctgttggtaggatcaggtgaggccttgcacattgcatgatgtactcgtcatggtgtgttggtgagaggatggatgtggatggatgtgtattctcagacgtagaccttgactggaggcttgatcgagagagtgggccgtattctttgagaggggaggctcgtgccagaaatggtgagtggatgactgtgacgctgtacattgcaggcaggtgagatgcactgtctcgattgtaaaatacattcagatgcaaaaaaaaaaaaaaaaaaaaaaa

SEQ ID NO: 32 - protein, 322 aa, Haematococcus pluvialis
TFHKPVSGASALPHIGPPPHLHRSFAATTMLSKLQSISVKARRVELARDITRPKVCLHAQRCSLVRLRVAAPQTEEALGTVQAAGAGDEHSADVALQQLDRAIAERRARRKREQLSYQAAAIAASIGVSGIAIFATYLRFAMHMTVGGAVPWGEVAGTLLLVVGGALGMEMYARYAHKAIWHESPLGWLLHKSHHTPRTGPFEANDLFAIINGLPAMLLCTFGFWLPNVLGAACFGAGLGITLYGMAYMFVHDGLVHRRFPTGPIAGLPYMKRLTVAHQLHHSGKYGGAPWGMFLGPQELQHIPGAAEEVERLVLELDWSKR

SEQ ID NO: 33 - DNA, 528 bp, Erwinia uredovora
  CDS (1)..(528)
atgttgtggatttggaatgccctgatcgttttcgttaccgtgattggcatggaagtgattgctgcactggcacacaaatacatcatgcacggctggggttggggatggcatctttcacatcatgaaccgcgtaaaggtgcgtttgaagttaacgatctttatgccgtggtttttgctgcattatcgatcctgctgatttatctgggcagtacaggaatgtggccgctccagtggattggcgcaggtatgacggcgtatggattactctattttatggtgcacgacgggctggtgcatcaacgttggccattccgctatattccacgcaagggctacctcaaacggttgtatatggcgcaccgtatgcatcacgccgtcaggggcaaagaaggttgtgtttcttttggcttcctctatgcgccgcccctgtcaaaacttcaggcgacgctccgggaaagacatggcgctagagcgggcgctgccagagatgcgcagggcggggaggatgagcccgcatccgggaagtaa

SEQ ID NO: 34 - protein, 175 aa, Erwinia uredovora
MLWIWNALIVFVTVIGMEVIAALAHKYIMHGWGWGWHLSHHEPRKGAFEVNDLYAVVFAALSILLIYLGSTGMWPLQWIGAGMTAYGLLYFMVHDGLVHQRWPFRYIPRKGYLKRLYMAHRMHHAVRGKEGCVSFGFLYAPPLSKLQATLRERHGARAGAARDAQGGEDEPASGK

SEQ ID NO: 35 - DNA, 1520 bp, Artificial Sequence
  Promotor
ctcgagtaccgaggcggaacggcaggaatgtttccctctcttttagagggcaattctttatccaatgtcatgttgatgctagatatttctgtctcttataataaggcgaatacccatttttgaattgaagttgagataaaaaaaaagggggcccaatttgtcaacgccaaagagtcaagctttttctttggctttagccgaacaatctaagacttattgtttttgaagatatttgaccttttctagatattccttcaagtaaagcttttttcgagttttttttttttttctttgtgaaggatttattgttattggtatccattttttattggaagacaagataagttaatattgattttgcttaaagattaaaaggaaatcagaaaacgacaataaaaaatgtaacggacaaactatggtgtcgattataagtctaaatccttaaaaaatgacaacgagttgctttcctctgaaaacaattcttttgtctttgcaagaaaggtttcttttttgtttgcttgcattacttaaacatcaaatcaaatgaaaggaataaagcagatttgagggcgaataaggattttctggtcaacaagatgtgagtgacacctaaggaactaaatgccattcatttgttttaaaacgacatcaaagattgatgatcaacaggattgagagagagaaaaagaactcgtgtcatttatttctgttgactgaaattttatatttagaaaaaatgtcaaatctatagctttagctatattacataacatttgaaataataataataaaaaaagacacattagagacacttttcaaactctaaataactgtctataaacacaaagaaaacaaagacctctataacaacttattagatttttctcgtacttttgtctaaagatgatgtattcttgttatcccacacttctttcatttgttcttgatgctactaaatatacaaaatttcttttttgcaagagatattattccaaaaattttcaaaaagaaatttttttcacaatagcagttgatcgtgtaacccaaagaggttctttgttattttgcacttccgctttgcggtgatgcatattcaaagtaatatatggaataaacaacgtgtttaagcatgaaagaaaggaaacaaaggccgctttgaacaaatgcataatatttcagacaaaaatgatctaaagcaagcagtaaatcaaacaagaaacattgctgattcgcgttagaaaacgataaaagtctaataagccactaagtatacttcaatgaactttttgtatgcttatggtccaatcagaccaataatttgtgaccattcctgaggtggctttggtgatgcggaaacagaaaaaaattttctcaccaatcgatttaaaaaacaatttctgctttgaaccaaaactttttttttctctttaatcattaactttatcaagtatgtacctaccctcaaagtcctcactcaagcacaattatgctaacattgttccaccttctctttagaaatg

SEQ ID NO: 36 - DNA, 16245 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttaatctatacaatgctccatagactcacattgatattgtcgaagatttcgatgctgacttagtagagcaactacaaaagttagcagagaagcatgatttcttaatctttgaagaccgcaagtttgcagatatcggtatgtgaattctatctattttttttctgatgtgtgcatggatgactcatgatcatattcttaggtaatactgtcaagcatcaatatggcaagggcgtttacaagattgcttcttggtctcatattactaatgctcacacagttcctggagaaggtattatcaagggacttgccgaagtcggcctccctcttggtcgtggcttgcttttgctagcagaaatgtcatctcaaggtgcattaactaagggtatttacactgccgaatctgtcaatatggctcgccgcaacaaagatttcgtttttggctttattgcacaacacaaaatgaatcagtatgatgatgaggattttgttgtcatgtcgcctgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 37 - DNA, 17877 bp, Artificial Sequence
  Promotor
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttttttcgagttttttttttttttctttgtgaaggatttattgttattggtatccattttttattggaagacaagataagttaatattgattttgcttaaagattaaaaggaaatcagaaaacgacaataaaaaatgtaacggacaaactatggtgtcgattataagtctaaatccttaaaaaatgacaacgagttgctttcctctgaaaacaattcttttgtctttgcaagaaaggtttcttttttgtttgcttgcattacttaaacatcaaatcaaatgaaaggaataaagcagatttgagggcgaataaggattttctggtcaacaagatgtgagtgacacctaaggaactaaatgccattcatttgttttaaaacgacatcaaagattgatgatcaacaggattgagagagagaaaaagaactcgtgtcatttatttctgttgactgaaattttatatttagaaaaaatgtcaaatctatagctttagctatattacataacatttgaaataataataataaaaaaagacacattagagacacttttcaaactctaaataactgtctataaacacaaagaaaacaaagacctctataacaacttattagatttttctcgtacttttgtctaaagatgatgtattcttgttatcccacacttctttcatttgttcttgatgctactaaatatacaaaatttcttttttgcaagagatattattccaaaaattttcaaaaagaaatttttttcacaatagcagttgatcgtgtaacccaaagaggttctttgttattttgcacttccgctttgcggtgatgcatattcaaagtaatatatggaataaacaacgtgtttaagcatgaaagaaaggaaacaaaggccgctttgaacaaatgcataatatttcagacaaaaatgatctaaagcaagcagtaaatcaaacaagaaacattgctgattcgcgttagaaaacgataaaagtctaataagccactaagtatacttcaatgaactttttgtatgcttatggtccaatcagaccaataatttgtgaccattcctgaggtggctttggtgatgcggaaacagaaaaaaattttctcaccaatcgatttaaaaaacaatttctgctttgaaccaaaactttttttttctctttaatcattaactttatcaagtatgtacctaccctcaaagtcctcactcaagcacaattatgctaacattgttccaccttctctttagaaatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 38 - DNA, 17238 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttctaccgcttggaccagtccagttccaggaccagtcgctccacctcctccgccgcacctggaatgtgctgcagctcctgtggacccaagaacataccccagggcgcgccaccgtacttgccgctgtggtgtagctggtgggccactgtcaggcgcttcatgtagggcaggccagcgatgggcccggtgggaaagcgcctgtgcaccaggccatcgtgtacaaacatatatgccatgccgtatagcgtgatgcccagccccgctccaaagcaggccgcccccaggacgttgggcagccagaagccaaaggtacacaggagcatggcgggcagtccattgatgattgcaaacaagtcgttggcttcaaagggtccagtgcgaggtgtgtggtggctcttgtgcagcagccagcccagaggcgactcatgccagatggctttgtgtgcatagcgggcatacatctccatgccgagcgcgccaccaaccaccaagaggagagtgccagccacttcaccccatggcactgcgccgcccacggtcatgtgcatggcaaatctcaggtaggtggcgaagatggcaatgcctgacacgccaattgatgctgcaatggcggcagcctggtatgacagctgctcccgtttgcgccgggcacgacgctctgcgatagcccggtcaagctgctggagtgctacatcggcgctgtgctcatcgcccgcgccggcagcctgcacggttcccagcgcctcctctgtctgtggtgctgccactcgcagccgaactaacgagcaccgctgagcatgcaggcagactttgggccgcgtgatgtcgcgggctagttcaacgcggcgggccttgacgctgattgactgcagcttcgacagcatagagataaaataaaaagagaagaaaagaaagtttgtacaatttctttttgtttatataacatacacgctatgtcaacatttagaataagggggaaaaaatcttccatcatattcgaatgcacaagattatttctttgttcgctctttttggtcgggtcatcgagatttagagtgtaatcaaagatactgtcatctcgagagcgttgcacaggctgctgtttgccaaattggatgtttgccgaattagtaaaatacgcaagcatttcttacctttccgctcccttttcctaattctcccaaagactaaatgaggaaagataaaggacaaagaaaatgtaaagacaaagaaattgaaaacgatataaacttgcagcacgtaagaccaaagcaaattggtaactattcttgtgtacaaacatgtataaaaaaaaacttttttttgctcctggaggacaaaatttcaaactccttgaagaagattgcttgtatatctatcatatgcatatatcatatcgatggaaaaagaaagtcaggcatgtatttataaaaagaagaatgtgccatgcttccgaatttcttttcactttcttttccttatctattttaatctcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 39 - DNA, 17238 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttagagataaaataaaaagagaagaaaagaaagtttgtacaatttctttttgtttatataacatacacgctatgtcaacatttagaataagggggaaaaaatcttccatcatattcgaatgcacaagattatttctttgttcgctctttttggtcgggtcatcgagatttagagtgtaatcaaagatactgtcatctcgagagcgttgcacaggctgctgtttgccaaattggatgtttgccgaattagtaaaatacgcaagcatttcttacctttccgctcccttttcctaattctcccaaagactaaatgaggaaagataaaggacaaagaaaatgtaaagacaaagaaattgaaaacgatataaacttgcagcacgtaagaccaaagcaaattggtaactattcttgtgtacaaacatgtataaaaaaaaacttttttttgctcctggaggacaaaatttcaaactccttgaagaagattgcttgtatatctatcatatgcatatatcatatcgatggaaaaagaaagtcaggcatgtatttataaaaagaagaatgtgccatgcttccgaatttcttttcactttcttttccttatctattttaatctcatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 40 - DNA, 18449 bp, Artificial Sequence
  Plasmid
gatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagattgtgactgatagcgagactctgggtcgatgttatctgcctcaacaatggcttagaaaagaagaaacagaacaaatacagcaaggcaacgcccgtagcctaggtgatcaaagactgttgggcttgtctctgaagcttgtaggaaaggcagacgctatcatggtgagagctaagaagggcattgacaagttgccggcaaactgtcaaggcggtgtacgagctgcttgccaagtatatgctgcaattggatctgtactcaagcagcagaagacaacatatcctacaagagctcatctaaaaggaagcgaacgtgccaagattgctctgttgagtgtatacaacctctatcaatctgaagacaagcctgtggctctccgtcaagctagaaagattaagagtttttttgttgattagtgaatttttgttttatttatgtctgatagttcaataaagagacaacacatacaatataaaatcattgtctttaaatgttaatttagtagagtgtaaagcctgcattttttttgtacgcataaacaatgaattcaccccgcttctggtttttaaataattatgtcaaactagggaaaattcttttttttctcttcgttctttttttggcttgttgtggagtcacaggcttgtcttcagattgatagaggttgtatacactcaacagagcaatcttggcacgttcgcttccttttagatgagctcttgtaggatatgttgtcttctgctgcttgagtacagatccaattgcagcatatacttggcaagcagctcgtacaccgccttgacagtttgccggcaacttgtcaatgcccttcttagctctcaccatgatagcgtctgcctttcctacaagcttcagagacaagcccaacagtctttgatcacctaggctacgggcgttgccttgctgtatttgttctgtttcttcttttctaagccattgttgaggcagataacatcgacccaacatcctcgagccatactacagcataaaaggatacgttttctttaacagaaatttacccttttgttatcagcacatacaaaaaaaaagaaatttaagatgagtaggacttccattctctcaaaaattttattcaatccataaatgaattatttttggacaaaaaagaaagattatgcctgattttctctattttttttttttttacaactccaccaatactttctagcccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggg

SEQ ID NO: 41 - DNA, 18449 bp, Artificial Sequence
  Plasmid
gatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcgggcgattgtgactgatagcgagactctgggtcgatgttatctgcctcaacaatggcttagaaaagaagaaacagaacaaatacagcaaggcaacgcccgtagcctaggtgatcaaagactgttgggcttgtctctgaagcttgtaggaaaggcagacgctatcatggtgagagctaagaagggcattgacaagttgccggcaaactgtcaaggcggtgtacgagctgcttgccaagtatatgctgcaattggatctgtactcaagcagcagaagacaacatatcctacaagagctcatctaaaaggaagcgaacgtgccaagattgctctgttgagtgtatacaacctctatcaatctgaagacaagcctgtggctctccgtcaagctagaaagattaagagtttttttgttgattagtgaatttttgttttatttatgtctgatagttcaataaagagacaacacatacaatataaaatcattgtctttaaatgttaatttagtagagtgtaaagcctgcattttttttgtacgcataaacaatgaattcaccccgcttctggtttttaaataattatgtcaaactagggaaaattcttttttttctcttcgttctttttttggcttgttgtggagtcacaggcttgtcttcagattgatagaggttgtatacactcaacagagcaatcttggcacgttcgcttccttttagatgagctcttgtaggatatgttgtcttctgctgcttgagtacagatccaattgcagcatatacttggcaagcagctcgtacaccgccttgacagtttgccggcaacttgtcaatgcccttcttagctctcaccatgatagcgtctgcctttcctacaagcttcagagacaagcccaacagtctttgatcacctaggctacgggcgttgccttgctgtatttgttctgtttcttcttttctaagccattgttgaggcagataacatcgacccaacatcctcgagccatactacagcataaaaggatacgttttctttaacagaaatttacccttttgttatcagcacatacaaaaaaaaagaaatttaagatgagtaggacttccattctctcaaaaattttattcaatccataaatgaattatttttggacaaaaaagaaagattatgcctgattttctctattttttttttttttacaactccaccaatactttctagcccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggg

SEQ ID NO: 42 - DNA, 17593 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttttttcgagttttttttttttttctttgtgaaggatttattgttattggtatccattttttattggaagacaagataagttaatattgattttgcttaaagattaaaaggaaatcagaaaacgacaataaaaaatgtaacggacaaactatggtgtcgattataagtctaaatccttaaaaaatgacaacgagttgctttcctctgaaaacaattcttttgtctttgcaagaaaggtttcttttttgtttgcttgcattacttaaacatcaaatcaaatgaaaggaataaagcagatttgagggcgaataaggattttctggtcaacaagatgtgagtgacacctaaggaactaaatgccattcatttgttttaaaacgacatcaaagattgatgatcaacaggattgagagagagaaaaagaactcgtgtcatttatttctgttgactgaaattttatatttagaaaaaatgtcaaatctatagctttagctatattacataacatttgaaataataataataaaaaaagacacattagagacacttttcaaactctaaataactgtctataaacacaaagaaaacaaagacctctataacaacttattagatttttctcgtacttttgtctaaagatgatgtattcttgttatcccacacttctttcatttgttcttgatgctactaaatatacaaaatttcttttttgcaagagatattattccaaaaattttcaaaaagaaatttttttcacaatagcagttgatcgtgtaacccaaagaggttctttgttattttgcacttccgctttgcggtgatgcatattcaaagtaatatatggaataaacaacgtgtttaagcatgaaagaaaggaaacaaaggccgctttgaacaaatgcataatatttcagacaaaaatgatctaaagcaagcagtaaatcaaacaagaaacattgctgattcgcgttagaaaacgataaaagtctaataagccactaagtatacttcaatgaactttttgtatgcttatggtccaatcagaccaataatttgtgaccattcctgaggtggctttggtgatgcggaaacagaaaaaaattttctcaccaatcgatttaaaaaacaatttctgctttgaaccaaaactttttttttctctttaatcattaactttatcaagtatgtacctaccctcaaagtcctcactcaagcacaattatgctaacattgttccaccttctctttagaaatgttgtggatttggaatgccctgatcgttttcgttaccgtgattggcatggaagtgattgctgcactggcacacaaatacatcatgcacggctggggttggggatggcatctttcacatcatgaaccgcgtaaaggtgcgtttgaagttaacgatctttatgccgtggtttttgctgcattatcgatcctgctgatttatctgggcagtacaggaatgtggccgctccagtggattggcgcaggtatgacggcgtatggattactctattttatggtgcacgacgggctggtgcatcaacgttggccattccgctatattccacgcaagggctacctcaaacggttgtatatggcgcaccgtatgcatcacgccgtcaggggcaaagaaggttgtgtttcttttggcttcctctatgcgccgcccctgtcaaaacttcaggcgacgctccgggaaagacatggcgctagagcgggcgctgccagagatgcgcagggcggggaggatgagcccgcatccgggaagtaagggcctgaccagaggcggccagcagcagcgttaatttttcgggcgtggtcgttgactgccgctgatcccaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 43 - DNA, 16954 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgttgtggatttggaatgccctgatcgttttcgttaccgtgattggcatggaagtgattgctgcactggcacacaaatacatcatgcacggctggggttggggatggcatctttcacatcatgaaccgcgtaaaggtgcgtttgaagttaacgatctttatgccgtggtttttgctgcattatcgatcctgctgatttatctgggcagtacaggaatgtggccgctccagtggattggcgcaggtatgacggcgtatggattactctattttatggtgcacgacgggctggtgcatcaacgttggccattccgctatattccacgcaagggctacctcaaacggttgtatatggcgcaccgtatgcatcacgccgtcaggggcaaagaaggttgtgtttcttttggcttcctctatgcgccgcccctgtcaaaacttcaggcgacgctccgggaaagacatggcgctagagcgggcgctgccagagatgcgcagggcggggaggatgagcccgcatccgggaagtaagggcctgaccagaggcggccagcagcagcgttaatttttcgggcgtggtcgttgactgccgctgatcccaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 44 - DNA, 16954 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttagagataaaataaaaagagaagaaaagaaagtttgtacaatttctttttgtttatataacatacacgctatgtcaacatttagaataagggggaaaaaatcttccatcatattcgaatgcacaagattatttctttgttcgctctttttggtcgggtcatcgagatttagagtgtaatcaaagatactgtcatctcgagagcgttgcacaggctgctgtttgccaaattggatgtttgccgaattagtaaaatacgcaagcatttcttacctttccgctcccttttcctaattctcccaaagactaaatgaggaaagataaaggacaaagaaaatgtaaagacaaagaaattgaaaacgatataaacttgcagcacgtaagaccaaagcaaattggtaactattcttgtgtacaaacatgtataaaaaaaaacttttttttgctcctggaggacaaaatttcaaactccttgaagaagattgcttgtatatctatcatatgcatatatcatatcgatggaaaaagaaagtcaggcatgtatttataaaaagaagaatgtgccatgcttccgaatttcttttcactttcttttccttatctattttaatctcatgttgtggatttggaatgccctgatcgttttcgttaccgtgattggcatggaagtgattgctgcactggcacacaaatacatcatgcacggctggggttggggatggcatctttcacatcatgaaccgcgtaaaggtgcgtttgaagttaacgatctttatgccgtggtttttgctgcattatcgatcctgctgatttatctgggcagtacaggaatgtggccgctccagtggattggcgcaggtatgacggcgtatggattactctattttatggtgcacgacgggctggtgcatcaacgttggccattccgctatattccacgcaagggctacctcaaacggttgtatatggcgcaccgtatgcatcacgccgtcaggggcaaagaaggttgtgtttcttttggcttcctctatgcgccgcccctgtcaaaacttcaggcgacgctccgggaaagacatggcgctagagcgggcgctgccagagatgcgcagggcggggaggatgagcccgcatccgggaagtaagggcctgaccagaggcggccagcagcagcgttaatttttcgggcgtggtcgttgactgccgctgatcccaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 45 - DNA, 19491 bp, Artificial Sequence
  Plasmid
agcttggtaccgagctcggatccactagtaacggccgccagtgtgctggaattcgcccttgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagggtgcggaaccaggcacgctggtttcacacctcatgcctgtgataaggtgtggctagagcgatgcgtgtgagacgggtatgtcacggtcgactggtctgatggccaatggcatcggccatgtctggtcatcacgggctggttgcctgggtgaaggtgatgcacatcatcatgtgcggttggaggggctggcacagtgtgggctgaactggagcagttgtccaggctggcgttgaatcagtgagggtttgtgattggcggttgtgaagcaatgactccgcccatattctatttgtgggagctgagatgatggcatgcttgggatgtgcatggatcatggtagtgcagcaaactatattcacctagggctgttggtaggatcaggtgaggccttgcacattgcatgatgtactcgtcatggtgtgttggtgagaggatggatgtggatggatgtgtattctcagacgtagaccttgactggaggcttgatcgagagagtgggccgtattctttgagaggggaggctcgtgccagaaatggtgagtggatgactgtgacgctgtacattgcaggcaggtgagatgcactgtctcgattgtaaaatacattcagatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgca

SEQ ID NO: 46 - DNA, 21300 bp, Artificial Sequence
  Plasmid
gatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgtcaatactcacttatctggaatttcatctctactatacactacctgtccttgcggcattgtgttggctgctaaagccgtttcactcacagcaagacaatctcaagtataaatttttaatgttgatggccgcctctaccgcatcgatttgggacaattatatcgtttatcatcgcgcttggtggtactgtcctacttgtgttgtggctgtcattggctatgtacctctagaagaatacatgttctttatcatcatgactttaatgactgtcgcgttctcaaactttgttatgcgttggcacttgcatactttctttattagacccaacacttcttggaagcaaacactattagtacgccttgtgcctgtttcagctttattggcaatcacttatcatgcttggcacttgacactgccaaataaaccttcattttatggttcatgcatcctttggtatgcttgtcctgtgttggctattctttggctgggtgctggcgaatatatcttgcgtcgacctgtggctgtccttttgtctattgttatccctagtgtatacctatgttgggctgatatcgtcgctattagtgctggcacatggcatatttctcttagaacaagcactggcaaaatggtagtacccgatttacctgtagaagaatgcctgttttttactttgatcaacacagtcttggtttttgctacctgtgctatagaccgcgctcaggccatcctccatgtgagcgcgcgtaatacgactcactatagggcgaattggagctccaccgcggtggcggccgctctagaactagtggatcccccgggctgcaggaattcggcacgagctacatttcacaagcccgtgagcggtgcaagcgctctgccccacatcggcccacctcctcatctccatcggtcatttgctgctaccacgatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcgggctcaggccatcctccatctgtacaaatcatctgttcaaaatcaaaaccctaaacaagccatttcccttttccagcatgtcaaagagctagcatgggccttctgtcttcctgaccaaatgctcaacaatgaattgtttgatgatcttactatcagctgggatattttacgtaaagcctcaaagtcattctatactgcatctgccgtttttccaagttatgtacgtcaagacttgggtgttctctatgctttctgcagagctaccgatgacctgtgcgatgatgaatccaaatctgttcaagaaagaagagaccaattagatcttactcgacaatttgttcgtgatctctttagccaaaagaccagtgcgcctattgtgattgattgggaattgtatcaaaaccaacttcctgcttcttgtatatcagcctttagagcctttactcgccttcgccatgtccttgaagtagaccctgtagaagaactattagatggttacaaatgggatcttgagcgtcgtcctatccttgatgaacaagacttggaggcatactctgcttgtgtggccagtagtgtgggtgaaatgtgcacacgtgtgattcttgctcaagaccaaaaggaaaatgatgcttggataattgaccgtgcacgtgagatggggctggtgctacaatacgttaacattgctcgagacattgtgactgatagcgagactctgggtcgatgttatctgcctcaacaatggcttagaaaagaagaaacagaacaaatacagcaaggcaacgcccgtagcctaggtgatcaaagactgttgggcttgtctctgaagcttgtaggaaaggcagacgctatcatggtgagagctaagaagggcattgacaagttgccggcaaactgtcaaggcggtgtacgagctgcttgccaagtatatgctgcaattggatctgtactcaagcagcagaagacaacatatcctacaagagctcatctaaaaggaagcgaacgtgccaagattgctctgttgagtgtatacaacctctatcaatctgaagacaagcctgtggctctccgtcaagctagaaagattaagagtttttttgttgattagtgaatttttgttttatttatgtctgatagttcaataaagagacaacacatacaatataaaatcattgtctttaaatgttaatttagtagagtgtaaagcctgcattttttttgtacgcataaacaatgagttcaccccgcttctggtttttaaataattatgtcaaactagggaaaattcttttttttctcttcgttctttttttggcttgttgtggagtcacaggcttgtcttcagattgatagaggttgtatacactcaacagagcaatcttggcacgttcgcttccttttagatgagctcttgtaggatatgttgtcttctgctgcttgagtacagatccaattgcagcatatacttggcaagcagctcgtacaccgccttgacagtttgccggcaacttgtcaatgcccttcttagctctcaccatgatagcgtctgcctttcctacaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggg

SEQ ID NO: 47 - DNA, 17756 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttcattttgctttgtaaatttctggtaactgccaccaagaaatatgaggatattcgtgatgttcctcgtggtagccaaaatgatagcacgtgataaatgaccaccaaataggacggctaattgtttgggcacaatgaggctgaacataaccccctattggttcactatggggtaaaaaagtaccaaaatagaataattgtaatgaacttaaaagcgagggtagcacccaaaagtaagttagattatcacttgggatatggagtatgtatttagcaaagttataaataatagtcaacgcaattatttgcccccaactccagtaacctttcataaaatgaaaataccaagcaaagaaactttggtgtttaccattgtgaaaatccgggtctattgagcttgctggattgtggtggtgtaaccaatgttttttcaatagtttttgatatggtaaaagaccataaagggatagggtcaatgttccaatcaaatgattaatcttggtgttttggggaaatactacgccatgcatggcatcatgagatgtaataaataatcccgtatataaaaatgtttgccatagtataacaggcaataacatccaaaattttagctttgagatgtcaagggaaagtaataaactcaggctaatgacccatgcgctaacaatgacaatagcaatgaaaagccccttaaactgagatttacttctcagtactggagtcagttttgcttgatgactgagtggttgttctaactggatcatttctaaagagaaggtggaacaatgttagcataattgtgcttgagtgaggactttgagggtaggtacatacttgataaagttaatgattaaagagaaaaaaaaagttttggttcaaagcagaaattgttttttaaatcgattggtgagaaaatttttttctgtttccgcatcaccaaagccacctcaggaatggtcacaaattattggtctgattggaccataagcatacaaaaagttcattgaagtatacttagtggcttattagacttttatcgttttctaacgcgaatcagcaatgtttcttgtttgatttactgcttgctttagatcatttttgtctgaaatattatgcatttgttcaaagcggcctttgtttcctttctttcatgcttaaacacgttgtttattccatatattactttgaatatgcatcaccgcaaagcggaagtgcaaaataacaaagaacctctttgggttacacgatcaactgctattgtgaaaaaaatttctttttgaaaatttttggaataatatctcttgcaaaaaagaaattttgtatatttagtagcatcaagaacaaatgaaagaagtgtgggataacaagaatacatcatctttagacaaaagtacgagaaaaatctaataagttgttatagaggtctttgttttctttgtgtttatagacagttatttagagtttgaaaagtgtctctaatgtgtctttttttattattattatttcaaatgttatgtaatatagctaaagctatagatttgacattttttctaaatataaaatttcagtcaacagaaataaatgacacgagttctttttctctctctcaatcctgttgatcatcaatctttgatgtcgttttaaaacaaatgaatggcatttagttccttaggtgtcactcacatcttgttgaccagaaaatccttattcgccctcaaatctgctttattcctttcatttgatttgatgtttaagtaatgcaagcaaacaaaaaagaaacctttcttgcaaagacaaaagaattgttttcagaggaaagcaactcgttgtcattttttaaggatttagacttataatcgacaccatagtttgtccgttacattttttattgtcgttttctgatttccttttaatctttaagcaaaatcaatattaacttatcttgtcttccaataaaaaatggataccaataacaataaatccttcacaaagaaaaaaaaaaaaaactcgaaaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 48 - DNA, 17118 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgatccagttagaacaaccactcagtcatcaagcaaaactgactccagtactgagaagtaaatctcagtttaaggggcttttcattgctattgtcattgttagcgcatgggtcattagcctgagtttattactttcccttgacatctcaaagctaaaattttggatgttattgcctgttatactatggcaaacatttttatatacgggattatttattacatctcatgatgccatgcatggcgtagtatttccccaaaacaccaagattaatcatttgattggaacattgaccctatccctttatggtcttttaccatatcaaaaactattgaaaaaacattggttacaccaccacaatccagcaagctcaatagacccggattttcacaatggtaaacaccaaagtttctttgcttggtattttcattttatgaaaggttactggagttgggggcaaataattgcgttgactattatttataactttgctaaatacatactccatatcccaagtgataatctaacttacttttgggtgctaccctcgcttttaagttcattacaattattctattttggtacttttttaccccatagtgaaccaatagggggttatgttcagcctcattgtgcccaaacaattagccgtcctatttggtggtcatttatcacgtgctatcattttggctaccacgaggaacatcacgaatatcctcatatttcttggtggcagttaccagaaatttacaaagcaaaatagaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 49 - DNA, 18449 bp, Artificial Sequence; identical to SEQ ID NO: 41
  Plasmid

SEQ ID NO: 50 - DNA, 18617 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgatccagttagaacaaccactcagtcatcaagcaaaactgactccagtactgagaagtaaatctcagtttaaggggcttttcattgctattgtcattgttagcgcatgggtcattagcctgagtttattactttcccttgacatctcaaagctaaaattttggatgttattgcctgttatactatggcaaacatttttatatacgggattatttattacatctcatgatgccatgcatggcgtagtatttccccaaaacaccaagattaatcatttgattggaacattgaccctatccctttatggtcttttaccatatcaaaaactattgaaaaaacattggttacaccaccacaatccagcaagctcaatagacccggattttcacaatggtaaacaccaaagtttctttgcttggtattttcattttatgaaaggttactggagttgggggcaaataattgcgttgactattatttataactttgctaaatacatactccatatcccaagtgataatctaacttacttttgggtgctaccctcgcttttaagttcattacaattattctattttggtacttttttaccccatagtgaaccaatagggggttatgttcagcctcattgtgcccaaacaattagccgtcctatttggtggtcatttatcacgtgctatcattttggctaccacgaggaacatcacgaatatcctcatatttcttggtggcagttaccagaaatttacaaagcaaaatagaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 51 - DNA, 18333 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgttgtggatttggaatgccctgatcgttttcgttaccgtgattggcatggaagtgattgctgcactggcacacaaatacatcatgcacggctggggttggggatggcatctttcacatcatgaaccgcgtaaaggtgcgtttgaagttaacgatctttatgccgtggtttttgctgcattatcgatcctgctgatttatctgggcagtacaggaatgtggccgctccagtggattggcgcaggtatgacggcgtatggattactctattttatggtgcacgacgggctggtgcatcaacgttggccattccgctatattccacgcaagggctacctcaaacggttgtatatggcgcaccgtatgcatcacgccgtcaggggcaaagaaggttgtgtttcttttggcttcctctatgcgccgcccctgtcaaaacttcaggcgacgctccgggaaagacatggcgctagagcgggcgctgccagagatgcgcagggcggggaggatgagcccgcatccgggaagtaagggcctgaccagaggcggccagcagcagcgttaatttttcgggcgtggtcgttgactgccgctgatcccaaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgatccagttagaacaaccactcagtcatcaagcaaaactgactccagtactgagaagtaaatctcagtttaaggggcttttcattgctattgtcattgttagcgcatgggtcattagcctgagtttattactttcccttgacatctcaaagctaaaattttggatgttattgcctgttatactatggcaaacatttttatatacgggattatttattacatctcatgatgccatgcatggcgtagtatttccccaaaacaccaagattaatcatttgattggaacattgaccctatccctttatggtcttttaccatatcaaaaactattgaaaaaacattggttacaccaccacaatccagcaagctcaatagacccggattttcacaatggtaaacaccaaagtttctttgcttggtattttcattttatgaaaggttactggagttgggggcaaataattgcgttgactattatttataactttgctaaatacatactccatatcccaagtgataatctaacttacttttgggtgctaccctcgcttttaagttcattacaattattctattttggtacttttttaccccatagtgaaccaatagggggttatgttcagcctcattgtgcccaaacaattagccgtcctatttggtggtcatttatcacgtgctatcattttggctaccacgaggaacatcacgaatatcctcatatttcttggtggcagttaccagaaatttacaaagcaaaatagaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 52 - DNA, 17 bp, Artificial Sequence
  Primer
gcngarggnathtggta

SEQ ID NO: 53 - DNA, 20 bp, Artificial Sequence
  Primer
tcngcnagraadatrttrtg

SEQ ID NO: 54 - DNA, 27 bp, Artificial Sequence
  Primer
aagtgacaccggttacacgcttgtctt

SEQ ID NO: 55 - DNA, 27 bp, Artificial Sequence
  Primer
gcttatcaccatctgttacctccttgc

SEQ ID NO: 56 - DNA, 32 bp, Artificial Sequence
  Primer
agagagggatccttaaatgcgaatatcgttgc

SEQ ID NO: 57 - DNA, 32 bp, Artificial Sequence
  Primer
agagagggatccatgtctgatcaaaagaagca

SEQ ID NO: 58 - DNA, 37 bp, Artificial Sequence
  Primer
actttattggatccttaaatgcgaatatcgttgctgc

SEQ ID NO: 59 - DNA, 38 bp, Artificial Sequence
  Primer
gttccaattggccacatgaagagtaagacaggaaacag

SEQ ID NO: 60 - DNA, 38 bp, Artificial Sequence
  Primer
cctgtcttactcttcatgtggccaattggaaccaacac

SEQ ID NO: 61 - DNA, 38 bp, Artificial Sequence
  Primer
ctattttaatcatatgtctgatcaaaagaagcatattg

SEQ ID NO: 62 - DNA, 16103 bp, Artificial Sequence
  Primer
gatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttgagtctatcgcctccaaaaagtacggtgctgaattcagatatcaatcgcctgttgctaaaattaacactgtcgataaagacaagcgtgtaaccggtgtcactttggaaagcggagaagtcattgaagccgatgcagtcgtatgtaatgcggatcttgtttatgcttatcaccatctgttacctccttgcaattggacaaagaagacattagcctcaaagaaactcacttcatcatctatttcgttttattggtccatgtcaacaaaggtgcctcaattagacgtacacaatatcttcttggctgaagcctacaaggaaagttttgatgagattttcaacgacttcggtttgccctctgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggg

SEQ ID NO: 63 - DNA, 25 bp, Artificial Sequence
  Primer
ggcgtacttgaaggaacccttaccg

SEQ ID NO: 64 - DNA, 25 bp, Artificial Sequence
  Primer
attgatgctcccggtcaccgtgatt

SEQ ID NO: 65 - DNA, 500 bp, Blakeslea trispora
aatctatacaatgctccatagactcacattgatattgtcgaagatttcgatgctgacttagtagagcaactacaaaagttagcagagaagcatgatttcttaatctttgaagaccgcaagtttgcagatatcggtatgtgaattctatctattttttttctgatgtgtgcatggatgactcatgatcatattcttaggtaatactgtcaagcatcaatatggcaagggcgtttacaagattgcttcttggtctcatattactaatgctcacacagttcctggagaaggtattatcaagggacttgccgaagtcggcctccctcttggtcgtggcttgcttttgctagcagaaatgtcatctcaaggtgcattaactaagggtatttacactgccgaatctgtcaatatggctcgccgcaacaaagatttcgtttttggctttattgcacaacacaaaatgaatcagtatgatgatgaggattttgttgtcatgtcgcctg

SEQ ID NO: 66 - DNA, 611 bp, Blakeslea trispora
gagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctct

SEQ ID NO: 67 - DNA, 720 bp, Blakeslea trispora
atgtcaatactcacttatctggaatttcatctctactatacactacctgtccttgcggcattgtgttggctgctaaagccgtttcactcacagcaagacaatctcaagtataaatttttaatgttgatggccgcctctaccgcatcgatttgggacaattatatcgtttatcatcgcgcttggtggtactgtcctacttgtgttgtggctgtcattggctatgtacctctagaagaatacatgttctttatcatcatgactttaatgactgtcgcgttctcaaactttgttatgcgttggcacttgcatactttctttattagacccaacacttcttggaagcaaacactattagtacgccttgtgcctgtttcagctttattggcaatcacttatcatgcttggcacttgacactgccaaataaaccttcattttatggttcatgcatcctttggtatgcttgtcctgtgttggctattctttggctgggtgctggcgaatatatcttgcgtcgacctgtggctgtccttttgtctattgttatccctagtgtatacctatgttgggctgatatcgtcgctattagtgctggcacatggcatatttctcttagaacaagcactggcaaaatggtagtacccgatttacctgtagaagaatgcctgttttttactttgatcaacacagtcttggtttttgctacctgtgctatagaccgc

SEQ ID NO: 68 - DNA, 1089 bp, Blakeslea trispora
ctgtacaaatcatctgttcaaaatcaaaaccctaaacaagccatttcccttttccagcatgtcaaagagctagcatgggccttctgtcttcctgaccaaatgctcaacaatgaattgtttgatgatcttactatcagctgggatattttacgtaaagcctcaaagtcattctatactgcatctgccgtttttccaagttatgtacgtcaagacttgggtgttctctatgctttctgcagagctaccgatgacctgtgcgatgatgaatccaaatctgttcaagaaagaagagaccaattagatcttactcgacaatttgttcgtgatctctttagccaaaagaccagtgcgcctattgtgattgattgggaattgtatcaaaaccaacttcctgcttcttgtatatcagcctttagagcctttactcgccttcgccatgtccttgaagtagaccctgtagaagaactattagatggttacaaatgggatcttgagcgtcgtcctatccttgatgaacaagacttggaggcatactctgcttgtgtggccagtagtgtgggtgaaatgtgcacacgtgtgattcttgctcaagaccaaaaggaaaatgatgcttggataattgaccgtgcacgtgagatggggctggtgctacaatacgttaacattgctcgagacattgtgactgatagcgagactctgggtcgatgttatctgcctcaacaatggcttagaaaagaagaaacagaacaaatacagcaaggcaacgcccgtagcctaggtgatcaaagactgttgggcttgtctctgaagcttgtaggaaaggcagacgctatcatggtgagagctaagaagggcattgacaagttgccggcaaactgtcaaggcggtgtacgagctgcttgccaagtatatgctgcaattggatctgtactcaagcagcagaagacaacatatcctacaagagctcatctaaaaggaagcgaacgtgccaagattgctctgttgagtgtatacaacctctatcaatctgaagacaagcctgtggctctccgtcaagctagaaagattaagagtttttttgttgattag

SEQ ID NO: 69 - DNA, 611 bp, Blakeslea trispora
agagataaaataaaaagagaagaaaagaaagtttgtacaatttctttttgtttatataacatacacgctatgtcaacatttagaataagggggaaaaaatcttccatcatattcgaatgcacaagattatttctttgttcgctctttttggtcgggtcatcgagatttagagtgtaatcaaagatactgtcatctcgagagcgttgcacaggctgctgtttgccaaattggatgtttgccgaattagtaaaatacgcaagcatttcttacctttccgctcccttttcctaattctcccaaagactaaatgaggaaagataaaggacaaagaaaatgtaaagacaaagaaattgaaaacgatataaacttgcagcacgtaagaccaaagcaaattggtaactattcttgtgtacaaacatgtataaaaaaaaacttttttttgctcctggaggacaaaatttcaaactccttgaagaagattgcttgtatatctatcatatgcatatatcatatcgatggaaaaagaaagtcaggcatgtatttataaaaagaagaatgtgccatgcttccgaatttcttttcactttcttttccttatctattttaatctc

SEQ ID NO: 70 - DNA, 882 bp, Haematococcus pluvialis
atgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtag

SEQ ID NO: 71 - DNA, 528 bp, Erwinia uredovora; identical to SEQ ID NO: 33

SEQ ID NO: 72 - DNA, 762 bp, Nostoc sp. PCC73102
atgatccagttagaacaaccactcagtcatcaagcaaaactgactccagtactgagaagtaaatctcagtttaaggggcttttcattgctattgtcattgttagcgcatgggtcattagcctgagtttattactttcccttgacatctcaaagctaaaattttggatgttattgcctgttatactatggcaaacatttttatatacgggattatttattacatctcatgatgccatgcatggcgtagtatttccccaaaacaccaagattaatcatttgattggaacattgaccctatccctttatggtcttttaccatatcaaaaactattgaaaaaacattggttacaccaccacaatccagcaagctcaatagacccggattttcacaatggtaaacaccaaagtttctttgcttggtattttcattttatgaaaggttactggagttgggggcaaataattgcgttgactattatttataactttgctaaatacatactccatatcccaagtgataatctaacttacttttgggtgctaccctcgcttttaagttcattacaattattctattttggtacttttttaccccatagtgaaccaatagggggttatgttcagcctcattgtgcccaaacaattagccgtcctatttggtggtcatttatcacgtgctatcattttggctaccacgaggaacatcacgaatatcctcatatttcttggtggcagttaccagaaatttacaaagcaaaatga

SEQ ID NO: 73 - DNA, 617 bp, Haematococcus pluvialis
tagggtgcggaaccaggcacgctggtttcacacctcatgcctgtgataaggtgtggctagagcgatgcgtgtgagacgggtatgtcacggtcgactggtctgatggccaatggcatcggccatgtctggtcatcacgggctggttgcctgggtgaaggtgatgcacatcatcatgtgcggttggaggggctggcacagtgtgggctgaactggagcagttgtccaggctggcgttgaatcagtgagggtttgtgattggcggttgtgaagcaatgactccgcccatattctatttgtgggagctgagatgatggcatgcttgggatgtgcatggatcatggtagtgcagcaaactatattcacctagggctgttggtaggatcaggtgaggccttgcacattgcatgatgtactcgtcatggtgtgttggtgagaggatggatgtggatggatgtgtattctcagacgtagaccttgactggaggcttgatcgagagagtgggccgtattctttgagaggggaggctcgtgccagaaatggtgagtggatgactgtgacgctgtacattgcaggcaggtgagatgcactgtctcgattgtaaaatacattcagatgc

SEQ ID NO: 74 - DNA, 1208 bp, Haematococcus pluvialis
attgtgactgatagcgagactctgggtcgatgttatctgcctcaacaatggcttagaaaagaagaaacagaacaaatacagcaaggcaacgcccgtagcctaggtgatcaaagactgttgggcttgtctctgaagcttgtaggaaaggcagacgctatcatggtgagagctaagaagggcattgacaagttgccggcaaactgtcaaggcggtgtacgagctgcttgccaagtatatgctgcaattggatctgtactcaagcagcagaagacaacatatcctacaagagctcatctaaaaggaagcgaacgtgccaagattgctctgttgagtgtatacaacctctatcaatctgaagacaagcctgtggctctccgtcaagctagaaagattaagagtttttttgttgattagtgaatttttgttttatttatgtctgatagttcaataaagagacaacacatacaatataaaatcattgtctttaaatgttaatttagtagagtgtaaagcctgcattttttttgtacgcataaacaatgaattcaccccgcttctggtttttaaataattatgtcaaactagggaaaattcttttttttctcttcgttctttttttggcttgttgtggagtcacaggcttgtcttcagattgatagaggttgtatacactcaacagagcaatcttggcacgttcgcttccttttagatgagctcttgtaggatatgttgtcttctgctgcttgagtacagatccaattgcagcatatacttggcaagcagctcgtacaccgccttgacagtttgccggcaacttgtcaatgcccttcttagctctcaccatgatagcgtctgcctttcctacaagcttcagagacaagcccaacagtctttgatcacctaggctacgggcgttgccttgctgtatttgttctgtttcttcttttctaagccattgttgaggcagataacatcgacccaacatcctcgagccatactacagcataaaaggatacgttttctttaacagaaatttacccttttgttatcagcacatacaaaaaaaaagaaatttaagatgagtaggacttccattctctcaaaaattttattcaatccataaatgaattatttttggacaaaaaagaaagattatgcctgattttctctattttttttttttttacaactccaccaatactttctag

SEQ ID NO: 75 - DNA, 6316 bp, Blakeslea trispora
  misc_feature (2694)..(2694): n is a, c, g, or t
aaggatgaagaatccaactctaataaaaatcttatggatatctttgatcgactcaaaaaggctttcaatgctattgctattaaaaaaaaagagagagagagaactatgagcaaaaggactctatgccaagatggcaaaaaggcaccagaaacccttagtttattattgcataatccagtcgagctagtacttctgtagctcaagcttaaccgaggatcttggaatcaactcgtctcgtcactcttgccgatgatcctagaaatggtatctatggatgttatactaacattgttatctttcaaggcctcgaagatgttattgttgcggtgataaataggctgctatgtactgaagttgctctgtaaaatgaatctagttcactgcctactcagcaaatggttgtttctaatgtctttaaagaaagaaaaaaagatacatatagactacccttcctttcaagactgtaatcgagaatcggccgatggtttattacaattagacgctgggaataagcaaaaggattcatctttgtaaataagagactggtgcatatgaaagcaaggatcgtatcaaggaatagttttgatcgagcatcaccagcaaatgctgctaatgttggcttcttctttgcttcctgagattgaatgggatgtgcctagagcattgctatttttaagtgtatactttagatttgtgtctttagatttgtgtcattttatttagtcaagaaagatccccctttctctatgtatgctaagaagaaggagcaagaagtgtatttacaagttggaatgagattgaaatattgtacataataataataaaaagaaaggtagatcaaaaaaaatgttctgcctattgtaagaaatcgggaccaacaggtgcttgataaccagaagtagcttccaattcaggtagaggctctagggacaaatacacaattatgacaggaattttcttgttgacttgaacactacaagagaaacgggtcagcacaaaatccgaaaaaaaaaagaaacggaccattcatgtcttacctatctagctctttgtcttcaattgcatcccattgctcaaccacagatacgcttcccaattgagtatattgatgaagtgttccctgcatttttcgcttgactaattccactacagtcacagtcttattaatgttttgtcctttaccagtcaggataatatgatctttttgcttcttctatcaaaaaaataattcttgttttgaataaaaaaaacaaatatttaaagaaactactttgatgacggtacctggaataactcgagacacacatctacatatgcgttgattttattgtggctaattcgaacctcattttctgctggtgggggctgttgactttcagttgctgagacgtccttcttgcttcttttatagtcttccactatgattttaatcaagaaagtaagtcagtgatgattgttacaagctatatatcttgaaaaagaacagagaggtattattatcagatgcaacatggttttctgtatcattttcatttcagtttctctgttcaaaaaaaaaaagaacactttctctttccactcctcaaattttttctgctaaactcctcgcaaaacatgtatttgctttaaactacaagttgcaattgtctgatttagcaatttcaatatgccttttgtgaatccacccaaaaataaacaagtgcttgagtatacttgggttcagttcaaaagaaagcaagcttttttttttctttcttgggaaagaaaaaaaaatattgttgagccatcctttaccagcagtatgcgagctacgacatagctggtctaacaatgactgcaagcaatagatcgagcttagtctttctattgcttcyttgtttgatctatgttcggccttacgctgacctatccaatactcgagataggcaacaagatttcgaacagtaatgaaataaatttcggataacagttgtggatgaggaagagaaagcgacttgaactcgagaaactttgttgaaatgaaatccgaccttttacgtgatcatcatgtattatcctctttttcttttttttcgtagtgaattacttactgattgcgctcaagtcgcgtctttataaagaagaaaaaaaaatattagaactttcaaaaaatataactgaaaataaaagtgtggctcggagagcaaataccacatcctttgtcttcgctttggtaacacggttaataagccactataggtgaataatgatcatttctgagaataaagcgcggcttgaagcttatatccatatcaggattcatattaggcacaactcacaattgaggttccagaagtgccaatttttttttcctgatagcctgtccaattaagatcaaaaaccactgagttttctctatatatttttttttttcataattcttaactcttcttcctctctctctctctctctctctttttggcttgcaaaaaaaatctttagtaataccaaagaaagcaaaccttttccttttcttatttccttgcttgttttttaatttttgatttctctatgctttaaatacccatttctttctttcttctgctattacctatcttttcattcctctcccccctctctctcttggtctataaacatcatgaagtcctcttttaaaagttcgcttgacatttatgctgtttatatacagcatcntgtgttttccaagtggttcattcttgcttttgttctttcgattttcctcaacacttatctactgaacgcttcgaagcaacagcccaaagtgataatcaaaaaggttattgagcgggtagaagtaccaagtagagaacaacctaaatcagtcataaagccctcctccaagaaacactcttctcatcatcagtctgatgtcattcgccctcttgatgaagtattgggtttgctcggaacacccgaggccttgactgatgaagagatcatctctattgttcaagctggtaaaatggccccctatgctcttgaaaaggtcttgggcgatttagagcgcgctgtccatatccgtcgtgctttgatctcccgtgactctcgtacgaaaactttggaagacagtatgcttcccgtgaaaaactatcattatgataaagtcatgggtgcttgttgtgaaaatgtcattggttatatgcctattccagtaggtgtcgcaggtaagaagttcaacaagtcgcgatatttgacaagttgctcatcattttcgaaacaggtcctttggtgattgatggtgattctattcatattcccatggcaactacggaaggttgtttagttgcttctactgccagaggttgtaaagcaatcaatgctggtggtggtgccaacacaattgttgttgctgatggtatgactcgaggtccttgtgtcgaatttcctacaatcactcgcgctgctgactgtaaacgatggattgaacaagagggtgaagctatcgtgaccgaggcattcaattcaacttctcgttttgctcgtgttcgtaaattgaaagttgctcttgccggtcgtctagtctacatccgtttctctaccactacaggtgatgcaatgggcatgaacatgatctccaagggttgtgaaaaggctttaagcaagattgctgagagatatcctgatatgcagatcatttctctttctggtaactattgtactgacaagaaacctgctgctatcaactggattgaaggacgtggtaaatctgttgttgctgasgctgtcatccctggtacggttgtcgaaaaggtattgaagacctctgttagtgctttggttgagctgaacatctctaaaaacctggttggttctgctatggctggctccgtcggtggctttaacgctcatgctgctaatattctaactgccatttaccttgctactggtcaagatcctgctcaaaatgtasagagttctaactgtattactttgatgaaagctgtcaatggcgaaagagaccttcatatctcttgtacaatgccctgtattgaagtaggcaccattggtggtggtactattttgcctcctcaacaagccatgttggatttcattggtgtgcgtggtcctcaccctaccgaacctggtgccaatgcccgwcgccttgctcgtgttatctgtgcctctgtgatggctggtgaattgtctttatgtgcagctttggctgctggtcatcttgtaaaggcacacatggctcataatcgtaataccactgctgctgccgctgttgttcctgcccctaanggcatagttgatgtctctacacctcctgctacacctgcagaaaagaatgatcctattcctggaagttgtatcaagtcatagaattaatattatatatatatcatatacaaaaaaaagaaaaaaaaaacactacatctatttatatttctccatgtacacacacacacacacatataaaaactctttattttccaatattttgcttttataaataatcttatttcattctaaataaactgtttttttttattaatcatcaaaccctgctgagagctgtgcaatatcatctatgttttcatggtttaactctggtatcggwcgagcctcctctgtacttgaagtttgtaggcagtttttatttaaggctgctggtcgatcatgatcatcakcaaacctgacagcatgaagttttgactgatgagcaatttcactaagggcagaatctgaactctttcgcttcctactattgaccatattgtctttaggtggaatgagtgaatagcgtcttgtcatatgtaacacagaatcaacaatatcctggtgatgaaactcggccaaacatagcgcctttctcccccaacaattataataatcaaaatgagaatgacatgtacggttttcctcgatgacaatatccaacgtcttgtcataatcctctgtgcgyataccattcatcttttggaagaacgcacggtagctctcacaagctgtcctcagagagttccgtgccatgtttcccaatgctcctggcaagtcgaaatgaagttgtcgaatctggcgatgtatgtctacaatgtcgcctgtttctttcattagatcaagcattcgtgtagcccaaatgatgtctatgttatgattttctttcattccagtaataactatagtttctcggcaaatcgaatgastgatggagtaaattcatcaaaagtgcaagtaatacatacagtgcttgaagaaatcttgtgtagcacgcctatattatgtaatataggatcgattctcgaaactcgacataaccaccaggctttagcaagcgttttatttcattcatgacaagctattgttaattcytgcttaataaaacaaaatgaaaaaaacatacccccctcmaaacttacttcccactcttgattggaaaaacaggtatagacgtgacgcatatgtatataatcaaaacactcatcaggatagggtaaaccattgagcacatcgcattgggtgaagaaagtattaggaggcttgatggctgtaggatatataggtgcaatatcaataccgtaaaactcagcatttgggaattctgtagccatctccagaatccaagtacctgtgccacaagcaacatcaagcactttaggtaagggtatacattgttgttcttgttgttgttgttgacaatcacttgagtctgagtttcgttttgattgttttaatgacaataattcttttacaggtgctgagaaattaccgtcaaatagatacttgtaaataaaatgctaaaaataaaaacaatagaaaaaaaaattgacgctcatttcattactatggaaataactgcaaaatcttaccacttgtacaagtctatcttgctcaatctcatcgtttggcagaatgtatttattgttgtagtattgatatcttctaccattcatgatataactgtcgcttctaatgctctgaggtgaagtacttgtaggtgaaggtggaagtgacgcaattttgtcaagcttaacaggatcctctcggctacatgttttctgcatatcaggaaaatcttgtttatttgaaacatcaacagtagatgtggtgtgatcttttttgaaaatatcgatgccttcctttgaaagccttttgaaaggctcttttaacttttttgagtgagagctacccatgatagcttatgaagaattaaaaagaaaaaagcaaaaaaaattaaaaaaaaaaaaagtagcaaaaaattctgtcgtaattatacaagccaatcaaaatcgaaattcatgcaaggcatagatgttcacgtggatttgatggttgatcctttttttttgcaaga

SEQ ID NO: 76 - DNA, 1170 bp, Thermus thermophilus
atgaagcgcctttccctgagggaggcctggccctacctgaaagacctccagcaagatcccctcgccgtcctgctggcgtggggccgggcccacccccggctcttccttcccctgccccgcttccccctggccctgatctttgaccccgagggggtggagggggcgctcctcgccgaggggaccaccaaggccaccttccagtaccgggccctctcccgcctcacggggaggggcctcctcaccgactggggggaaagctggaaggaggcgcgcaaggccctcaaagaccccttcctgccgaagaacgtccgcggctaccgggaggccatggaggaggaggcccgggccttcttcggggagtggcggggggaggagcgggacctggaccacgagatgctcgccctctccctgcgcctcctcgggcgggccctcttcgggaagcccctctccccaagcctcgcggagcacgcccttaaggccctggaccggatcatggcccagaccaggagccccctggccctcctggacctggccgccgaagcccgcttccggaaggaccggggggccctctaccgcgaggcggaagccctcatcgtccacccgcccctctcccaccttccccgagagcgcgccctgagcgaggccgtgaccctcctggtggcgggccacgagacggtggcgagcgccctcacctggtcctttctcctcctctcccaccgcccggactggcagaagcgggtggccgagagcgaggaggcggccctcgccgccttccaggaggccctgaggctctacccccccgcctggatcctcacccggaggctggaaaggcccctcctcctgggagaggaccggctccccccgggcaccaccctggtcctctccccctacgtgacccagaggctccacttccccgatggggaggccttccggcccgagcgcttcctggaggaaagggggaccccttcggggcgctacttcccctttggcctggggcagaggctctgcctggggcgggacttcgccctcctcgagggccccatcgtcctcagggccttcttccgccgcttccgcctagaccccctccccttcccccgggtcctcgcccaggtcaccctgaggcccgaaggcgggcttcccgcgcggcctagggaggaggtgcgggcgtga

SEQ ID NO: 77 - DNA, 2981 bp, Blakeslea trispora
tctagaattcattccattcgaaaggatcaacataaccaatttaatgactactagctaatggatacaaatatacgcacaaaaaaagaaagaattctatgatcaaagagaacacagacacagagtgatacatttaaatggttaagttcttatgatgttaaaatggtaactttattattgaattaaatgcgaatatcgttgctgctttgtacttggaaaacgttaggtaaaagttggttaatgaaagaagcaggagttgtagtatcatctcttgggaagaaatagaaaaagaggaaagtaacaaagtaacaagcaagacaataatagatccaatggctttcggtcttacgagtttgttcaggagcatacttcttttggctatcttgtaactttcttggtaagggattctggccaaagcttttacagacttggtcggaagtaagcttacttccagcaagaacgataggaacaccagtacctggatgtgtactacaaagaaaagagaaatgagtacgtgcgttattaaaaaaaagaaaaaaagagggcaaaagtattacctagctccgacaaagaaaagattatcataacggtttgtggaatccttggtactaggtctgaaccagagaacttggaacacatcatgagaaagaccaagaatagaacctctccaaaggttaaacttgctttgccaaacactaggatcattcacttcttcatgttcaatcaaattagcaaagttgtttactcccaaacgacgttcgataacttccagaaccatcttgcgtgcacggtttaccaactcaggataattttcttcagcactgtttcctgtcttactcttcatatggccaattggaaccaacacaataatggagtccttgttgggaggtgcggcagattcatcaattcgagatggaacgttgacatagaatgaagcttcagagggcaaaccgaagtcgttgaaaatctcatcaaaactttccttgtaggcttcagccaagaagatattgtgtacgtctaattgaggcacctttgttgacatggaccaataaaacgaaatagatgatgaagtgagtttctttgaggctaatgtcttctttgtccaattgcaaggaggtaacagatggtgataagcataaacaagatccgcattacatacgactgcatcggcttcaatgacttctccgctttccaaagtgacaccggttacacgcttgtctttatcgacagtgttaattttagcaacaggcgattgatatctgaattcagcaccgtactttttggaggcgatagactcaagcttctgaacaaccatgttgaaaccaccacgaggataccagataccttcagcaaactcggtgtattgtaacaaactgtaaactgctggagcatcataaggcgacatactatattccaaaaatagaaaatagaacaatgaatatcaaaattcctttcacttgccctttttcacatttctcttttcccacccccgaccggtctcactcatttttttttcatcccacaccacgcgttgtatgtgtacttaccccatatacattgtttgaaaagtaaaagccatacgcattttcttggtttggaaatatttactggctcggtcatagatcttaccaaacaagtgcaagcgaaagatttcaggcacatactgaagacgaatcaaatcccaaatggtttcaaagttgcgcttgatagcaataaatgtaccttgttcataatggacatgtgtttccttcatgaaatccaagaatctaccaaatccaaggggaccctcaatacggtccaattcgcccttcatcttggttaaatcggaagagagttgtacggcatcaccgtcgtcaaaatgaaccttatagttattgtcacagcgaagcaaatccaaatgatcaccaatacgttcatccaaatcagcaaatgcatcttcaaaaagcttaggcatcaaatagagtgagggaccctgatcaaagcgatgaccatcgtgatgaatgaatgaacaacggccaccggaaaagtcgttcttttcaacaacagtaactcgaaaaccttcacgagcaagacgagcagcagtagcagttccgccaataccggcaccaatgacaacaatatgcttcttttgatcagacatgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgtcaatactcacttatctggaatttcatctctactatacactacctgtccttgcggcattgtgttggctgctaaagccgtttcactcacagcaagacaatctcaagtataaatttttaatgttgatggccgcctctaccgcatcgatttgggacaattatatcgtttatcatcgcgcttggtggtactgtcctacttgtgttgtggctgtcattggctatgtacctctaga

SEQ ID NO: 78 - DNA, 1749 bp, Blakeslea trispora
atgtctgatcaaaagaagcatattgttgtcattggtgccggtattggcggaactgctactgctgctcgtcttgctcgtgaaggttttcgagttactgttgttgaaaagaacgacttttccggtggccgttgttcattcattcatcacgatggtcatcgctttgatcagggtccctcactctatttgatgcctaagctttttgaagatgcatttgctgatttggatgaacgtattggtgatcatttggatttgcttcgctgtgacaataactataaggttcattttgacgacggtgatgccgtacaactctcttccgatttaaccaagatgaagggcgaattggaccgtattgagggtccccttggatttggtagattcttggatttcatgaaggaaacacatgtccattatgaacaaggtacatttattgctatcaagcgcaactttgaaaccatttgggatttgattcgtcttcagtatgtgcctgaaatctttcgcttgcacttgtttggtaagatctatgaccgagccagtaaatatttccaaaccaagaaaatgcgtatggcttttacttttcaaacaatgtatatgggtatgtcgccttatgatgctccagcagtttacagtttgttacaatacaccgagtttgctgaaggtatctggtatcctcgtggtggtttcaacatggttgttcagaagcttgagtctatcgcctccaaaaagtacggtgctgaattcagatatcaatcgcctgttgctaaaattaacactgtcgataaagacaagcgtgtaaccggtgtcactttggaaagcggagaagtcattgaagccgatgcagtcgtatgtaatgcggatcttgtttatgcttatcaccatctgttacctccttgcaattggacaaagaagacattagcctcaaagaaactcacttcatcatctatttcgttttattggtccatgtcaacaaaggtgcctcaattagacgtacacaatatcttcttggctgaagcctacaaggaaagttttgatgagattttcaacgacttcggtttgccctctgaagcttcattctatgtcaacgttccatctcgaattgatgaatctgccgcacctcccaacaaggactccattattgtgttggttccaattggccatatgaagagtaagacaggaaacagtgctgaagaaaattatcctgagttggtaaaccgtgcacgcaagatggttctggaagttatcgaacgtcgtttgggagtaaacaactttgctaatttgattgaacatgaagaagtgaatgatcctagtgtttggcaaagcaagtttaacctttggagaggttctattcttggtctttctcatgatgtgttccaagttctctggttcagacctagtaccaaggattccacaaaccgttatgataatcttttctttgtcggagctagtacacatccaggtactggtgttcctatcgttcttgctggaagtaagcttacttccgaccaagtctgtaaaagctttggccagaatcccttaccaagaaagttacaagatagccaaaagaagtatgctcctgaacaaactcgtaagaccgaaagccattggatctattattgtcttgcttgttactttgttactttcctctttttctatttcttcccaagagatgatactacaactcctgcttctttcattaaccaacttttacctaacgttttccaagtacaaagcagcaacgatattcgcatttaa

SEQ ID NO: 79 - DNA, 25 bp, Artificial Sequence
  Primer
ccgatggcgacgacggaaggttgtt

SEQ ID NO: 80 - DNA, 25 bp, Artificial Sequence
  Primer
catgttcatgcccattgcatcacct

(19) United States
(12) Patent Application Publication
(10) Pub. No.: US 2006/0234333 A1
(43) Pub. Date: Oct. 19, 2006

(54) Method for producing carotenoids or their precursors using genetically modified organisms of the blakeslea genus, carotenoids or their precursors produced by said method and use thereof

(75) Inventors: Markus Matuschek, Weinheim (DE); Daniela Klein, Mannhelm (DE); Thorsten Heinekamp, Hannover (DE); Andre Schmidt, Springe (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT Patents, Trademarks and Licenses, ludwigshafen (DE)

(21) Appl. No.: 10/541,750

(22) PCT Filed: Jan. 9, 2004
(86) PCT No.: PCT/EP04/00099

§ 371 Date: Sep. 6, 2005

(30) Foreign Application Priority Data
Jan. 9, 2003 (DE) 103 00 649.4
Sep. 8, 2003 (DE) 103 41 271.9

(51) Int. Cl. C12P 23/00; C12N 15/74; C12N 1/14

(52) U.S. Cl. 435/67; 435/254.1; 435/483

(57) ABSTRACT

The invention relates to a method for producing carotenoids or their precursors using genetically modified organisms of the Blakeslea genus. Said method comprises the following steps (i) transformation of at least one of the cells, (ii) optional homokaryotic conversion of the cells obtained in step (i) to produce cells, in which one or more genetic characteristics of the nuclei are all modified in an identical manner and said modification manifests itself in the cells, (iii) selection and reproduction of the genetically modified cell or cells, (iv) cultivation of the genetically modified cells, (v) preparation of the carotenoids produced by the genetically modified cells or the carotenoid precursor produced by said genetically modified cells. The invention also relates to carotenoids or their precursors produced according to said method and to the use thereof.

The invention relates to a method for producing carotenoids or their precursors using genetically modified organisms of the Blakeslea genus, to carotenoids or their precursors produced by said method and to the use and provision thereof, in particular as highly pure carctenoids, as foodstuffs comprising carotenoid-producing organisms and at least one carotenoid, in particular animal feedstuffs, animal feed supplements and food supplements, and to the use of the carotenoids obtainable by said method for producing cosmetic, pharmaceutical, dermatological preparations, foodstuffs or food supplements.

Blakeslea trispora is a known producer organism for β-carotene (Ciegler, 1965, Adv Appl Microbiol. 7:1) and lycopene (EP 1201762, EP 1184464, WO 03/038064).

Various DNA sequences of Blakeslea trispora are known already, in particular the DNA sequence coding for the genes of carotenoid biosynthesis from geranylgeranyl pyrophosphate to β-carotene (WO 03/027293).

The high productivity achieved by Blakeslea for producing of lycopene and β-carotene, in particular, renders this organism suitable for fermentative production of carotenoids.

It is also of interest to further increase the productivities of carotenes and their precursors which have previously been produced naturally and to enable further carotenoids such as, for example, xanthophylls to be produced which have been produced by and isolated from Blakeslea only to a very low extent, if at all, previously.

Carotenoids are added to feedstuffs, foodstuffs, food supplements, cosmetics and medicaments. Carotenoids are used especially as pigments for coloring. Aside from this, the antioxidative action of carotenoids and other properties of these substances are utilized. The carotenoids are divided into the pure hydrocarbons, the carotenes and the oxygen-containing hydrocarbons, the xanthophylls. Xanthophylls such as canthaxanthin and astaxanthin are employed, for example, in the pigmentation of hens' eggs and fish (Britton et al. 1998, Carotinoids, Vol. 3, Biosynthesis and Metabolism). The carotenes β-carotene and lycopene are employed especially in human nutrition. β-Carotene, for example, is used as a colorant for beverages. Lycopene has disease-preventing action (Argwal and Rao, 2000, CMAJ 163:739-744; Rao and Argwal 1999, Nutrition Research 19:305-323). The colorless carotenoid precursor phytoene is especially suitable for applications as antioxidant in cosmetic, pharmaceutical or dermatological preparations.

Most of the carotenoids and their precursors which are employed as additives in the abovementioned applications are prepared by chemical synthesis. Said chemical synthesis is very complicated and causes high production costs. In contrast, fermentative processes are comparatively simple and based on inexpensive starting materials. Fermentative processes to produce carotenoids and their precursors may be economically attractive and capable of competing with chemical synthesis, if the productivity of previous fermentative processes were increased or new carotenoids were able to be prepared on the basis of the known producer organisms.

This requires a genetically engineered, i.e. specific genetic, modification of Blakeslea, in particular if xanthophylls are to be produced, since these compounds are not naturally synthesized by the Blakeslea wild type.

For example, two previous methods are known for producing phytoene by means of fermentation of Blakeslea trispora:
(i) Random mutagenesis using chemical agents such as MNNG may generate mutants which cannot convert phytoene to lycopene, and thus further to β-carotene (Mehta and Cerdá-Olmedo, 1995, Appl. Microbiol. Biotechnol. 42:836-838).
(ii) Addition of inhibitors of the enzyme phytoene desaturase, such as, for example, diphenylamine and cinnamyl alcohol, can block further conversion of phytoene, causing the latter to accumulate (Cerdá-Olmedo, 1989, In: E. Vandamme, ed. Biotechnology of vitamin, growth factor and pigment production. London: Elsevier Applied Science, pp. 27-42).

The methods mentioned for preparing phytoene using Blakeslea trispora, however, have a number of disadvantages.

Said random mutagenesis usually affects not only the genes of carotenoid biosynthesis for further conversion of phytoene but also other important genes. For this reason, growth and synthetic performance of the mutants are often impaired. The generation of, for example, phytoene overproducers by random mutagenesis of lycopene overproducers or β-carotene overproducers can therefore be achieved only with great experimental complexity, if at all. The addition of inhibitors increases production costs and may cause a contamination of the product. In addition, cell growth may be impaired by the inhibitor, thus limiting production of carotenoids or their precursors, in particular phytoene.

The abovementioned disadvantages of random mutagenesis and addition of inhibitor could be avoided by a genetically engineered modification.

Thus far, however, no methods for the genetically engineered, i.e. specific genetic, modification of Blakeslea, in particular Blakeslea trispora, are known.

A method for the production of genetically modified fungi which has been successfully employed in some cases is Agrobacterium-mediated transformation. Thus, for example, the following organisms have been transformed by agrobacteria: Saccharomyces cerevisiae (Bundock et al., 1995, EMBO Journal, 14:3206-3214), Aspergillus awamori, Aspergillus nidulans, Aspergillus niger, Colletotrichum gloeosporioides, Fusarium solani pisi, Neurospora crassa, Trichoderma reesei, Pleurotus ostreatus, Fusarium graminearum (van der Toorren et al., 1997, EP 870835), Agraricus bisporus, Fusarium venenatum (de Groot et al., 1998, Nature Biotechnol. 16:839-842), Mycosphaerella graminicola (Zwiers et al. 2001, Curr. Genet. 39:388-393), Glarea lozoyensis (Zhang et al., 2003, Mol. Gen. Genomics 268:645-655), Mucor miehei (Monfort et al. 2003, FEMS Microbiology Lett. 244:101-106).

Of particular interest is a homologous recombination which involves as many sequence homologies as possible between the DNA to be introduced and the cellular DNA, so that it is possible to introduce or eliminate site-specifically genetic information in the genome of the recipient organism. Otherwise, the donor DNA will be integrated into the genome of the recipient organism by illegitimate or nonhomologous recombination which is not site-specific.

Agrobacterium-mediated transformation and subsequent homologous recombination of the transferred DNA have been detected previously for the following organisms: Aspergillus awamori (Gouka et al. 1999, Nature Biotech 17:598-601), Glarea lozoyensis (Zhang et al., 2003, Mol. Gen. Genomics 268:645-655), Mycosphaerella graminicola ((Zwiers et al. 2001, Curr. Genet. 39:388-393).

Another known method for transforming fungi is electroporation. Hill, Nucl. Acids. Res. 17:8011 has shown the integrative transformation of yeast by electroporation. Transformation of filamentous fungi has been described by Chakaborty and Kapoor (1990, Nucl. Acids. Res. 18:6737).

A “biolistic” method, i.e. the transfer of DNA by bombardment of cells with DNA-loaded particles, has been described, for example, for Trichoderma harzianum and Gliocladium virens (Lorito et al. 1993, Curr. Genet. 24:349-356).

However, it has not been possible previously to successfully employ these methods for specific genetic modification of Blakeslea and in particular Blakeslea trispora.

A particular difficulty in producing genetically modified Blakeslea and Blakeslea trispora is the fact that their cells are multinuclear at all stages of the sexual and vegetative cell cycles. For example, spores of the Blakeslea trispora strains NRRL2456 and NRRL2457 were found to have an average of 4.5 nuclei per spore (Metha and Cerdá-Olmedo, 1995, Appl. Microbiol. Biotechnol. 42:836-838). As a consequence of this, the genetic modification is usually present only in one or a few nuclei, i.e. the cells are heterokaryotic.

If the genetically modified Blakeslea, in particular Blakeslea trispora, are intended to be used for production, it is important, in particular in the case of gene deletion, that the genetic modification is present in all nuclei of the producer strains so as to make possible a stable and high synthetic performance without byproducts. The strains must consequently be homokaryotic with respect to said genetic modification.

A method of generating homokaryotic cells has been described only for Phycomyces blakesleeanus (Roncero et al., 1984, Mutat. Res. 125:195). According to the method described there, nuclei are eliminated in the cells by adding the mutagenic agent MNNG (N-methyl-N′-nitro-N-nitrosoguanidine) so as to obtain statistically a certain number of cells with only one functional nucleus. The cells are then subjected to a selection in which only mononuclear cells having a recessive selection marker can grow into a mycelium. The progeny of these selected cells are multinuclear and homokaryotic. An example of a recessive selection marker for Phycomyces blakesleanus is dar. dar⁺ strains absorb the toxic riboflavin analog 5-carbon-5-deazariboflavin, unlike dar⁻ strains (Delbrück et al. 1979, Genetics 92:27). Recessive mutants are selected by adding 5-carbon-5-deazariboflavin (DARF).

However, this method is unknown for Blakeslea, in particular Blakeslea trispora, and has in particular not been described in relation to a transformation or production of carotenoids or their precursors.

Isolation from natural resources is also carried out. A known example of obtaining phytoene is to extract a mixture of carotenoids, vitamin E and other components, which also contains phytoene, from tomatoes, carrots or palm oil etc. A problem here is the separation of the individual carotenoids from one another. Thus, for example, phytoene cannot be obtained in a pure form by this method. In particular, the naturally occurring amount of carotenoids in the plants is low.

In contrast, fermentative processes are comparatively simple and based on inexpensive starting materials. Fermentative processes to produce carotenoids may be economically attractive and capable of competing with chemical synthesis, if the productivity of previous fermentative processes were increased or new carotenoids were able to be prepared on the basis of the known producer organisms. A problem of the fermentative production of carotenoids, however, are the work-up processes which provide only small amounts of highly pure carotenoids. Moreover, they usually require processes with multiple steps, if appropriate with the use of large amounts of solvents. Thus, large amounts of waste are produced or a lot of effort has to go into the recycling process.

The production of carotenoids by various microorganisms is known per se. Thus, for example, WO 00/13654 A2 discloses the extraction of a mixture of phytoene and phytofluene from algae of the species Dunaliella sp. This method too, does not produce phytoene in a pure form, and the latter must be separated from the other products. Moreover, the algae are genetically unmodified and their biosynthesis must be influenced by means of an added inhibitor.

WO 98/03480 A1 also discloses Blakeslea trispora as producer organism for β-carotene. Here, β-carotene crystals are obtained from Blakeslea trispora biomass by means of extraction. However, the method described requires large amounts of different solvents in order to obtain crystals with high purity by several extraction and washing steps. The amounts of β-carotene obtained are also small based on the amount of biomass used.

WO 01/83437 A1 discloses a method for extracting astaxanthin from yeast, which comprises treating the culture broth with microwave radiation for sterilization and cell disruption. According to this, cell disruption by means of microwave radiation is required in order to obtain astaxanthin from yeast without destroying it. Subsequently, astaxanthin is to be extracted by means of methanol, ethanol or acetone or mixtures thereof. This, however, requires large amounts of solvent (5 to 20 parts of solvent to 1 part of suspension) and a long time (24 h). Moreover, astaxanthin purities are not indicated and the amounts obtained are small. However, experiments of the applicant and other publications confirm that extraction by means of methanol or ethanol is not possible.

WO 98/50574 likewise discloses the isolation of carotenoid crystals from a microorganism biomass, and here, in contrast to WO 01/83437 A1, it is possible to use methanol, ethanol, acetone only for removing lipids from the biomass, i.e. for washing. Accordingly, the solvent used for extracting carotenoids is ethyl acetate, hexane or an oil. Subsequently, a plurality of purification and washing steps with large amounts of ethanol and water are required, resulting in a purity of only 93.3% with a yield of 35%.

WO 03/038064 A2 describes the fermentative production of lycopene by cocultivation of mutated Blakeslea trispora mating type (−) and Blakeslea trispora mating type (+) which produce lycopene without addition of inhibitors of carotenoid biosynthesis. The mutant employed for fermentation is generated by nonselective chemical mutation and subsequent screening. The culture broth is worked up by means of cell disruption and subsequent purification with different aqueous media with varying salt content and pH and with water-immiscible organic solvents such as ethyl acetate, hexane and 1-butanol, in order to remove lipids. An extraction using large amounts of ethyl acetate is described as an alternative. Information about the purity is missing. Since ethyl acetate and hexane are solvents for lycopene, it can be assumed that part of the lycopene is washed out, thus reducing the theoretically possible yield.

WO 01/55100 A1 also describes the isolation of carotenoids in general, and specifically β-carotene, from the biomass by applying a plurality of washing and purification steps to the disrupted biomass without extraction by means of solvents. This involves washing disrupted Blakeslea trispora biomass with water, lye, acid, butanol and ethanol so that the use of a large number of different solvents and aqueous media is required. The purity of the β-carotene obtained is 96-98%. However, there is no information regarding the yield.

WO 97/36996 A2 generally describes a method for isolating substances (inter alia carotenoids) from microorganisms, said substances being isolated from the biomass by means of solid/liquid extraction. Cell disruption is apparently not required here but the biomass must first be rendered granulated and porous by extrusion. The possibility of isolating only carotenoids and information about their purity or yield are not indicated. The residue from the extrusion may subsequently be used as feed additive.

In all the methods described above, large amounts of solvent must be used for extraction, in order to increase the amount of isolated carotenoid by complete extraction, and/or large amounts of aqueous media must be used for purification and washing. This causes high costs and complicated recycling measures and, if appropriate, waste.

Moreover, the nutritious culture broth and the biomass present therein are treated as waste, after extraction or isolation of the carotenoids. Aside from these superficial disadvantages, the methods indicated above have another decisive disadvantage, namely the fact that the carotenoids must be added to the foodstuffs subsequently, i.e. they are not part of the foodstuffs per se or are present only in an insufficient amount. It would therefore be greatly advantageous if the carotenoid content in the foodstuffs would already be covered by the actual foodstuffs themselves.

Likewise, it is necessary to further increase the productivities of the previously naturally produced carotenes and their precursors and to enable further carotenoids such as, for example, xanthophylls, particularly preferably astaxanthin or zeaxanthin, and phytoene or bixin to be produced which have previously been produced by and isolated from the wild types of the microorganisms only to a very low extent, if at all.

It is an object of the invention to provide genetically modified cells of Blakeslea strains, in particular Blakeslea trispora, which produce carotenoids or their precursors, in particular xanthophylls, particularly preferably astaxanthin or zeaxanthin, and phytoene or bixin. Moreover, the method is intended to allow carotenoid productivity of the modified cells to be increased compared to the corresponding wild types. The method is also intended to allow new cells or mycelium composed thereof to be generated which are suitable for the use in the production of carotenoids or their precursors which were previously unobtainable from the naturally occurring fungi in economically interesting quantities, in particular xanthophylls, particularly preferably astaxanthin or zeaxanthin, and phytoene or bixin. In this context, the method is intended to enable Blakeslea strains, in particular Blakeslea trispora, to be genetically modified and to allow production of homokaryotic genetically modified producer strains.

Furthermore, the method is intended to enable further carotenoids such as, for example, xanthophylls, in particular astaxanthin or zeaxanthin, and phytoene or bixin to be produced which have previously been produced by and isolated from the wild types of the microorganisms only to a very low extent, if at all.

It is also an object of the present invention to make available a method for producing carotenoids from genetically modified cells of Blakeslea strains, in particular Blakeslea trispora, which method allows the use of smaller amounts of solvent and essentially does not produce waste and, moreover, allows high purity and higher yields.

In this connection, it is intended to utilize a very large portion of the nutrients present in the fermenter, both carotenoids and other nutrients present in the microorganisms.

Thus, it is also an object of the present invention to provide a method for producing a carotenoid-containing foodstuff which itself covers the carotenoid requirements without additives. In particular, the nutrient content of the foodstuffs obtainable by said method is intended to be at least equal to that of the previously obtainable foodstuffs. The method is also intended to enable the produced carotenoids to be utilized efficiently.

This object is achieved by a method for producing carotenoids or their precursors using genetically modified organisms of the Blakeslea genus, which method comprises the following steps:
(i) transformation of at least one of the cells,
(ii) optional homokaryotic conversion of the cells obtained in step (i) to produce cells in which one or more genetic characteristics of the nuclei are all modified in an identical manner and said genetic modification manifests itself in the cells, and
(iii) selection and reproduction of the genetically modified cell or cells,
(iv) cultivation of the genetically modified cells,
(v) preparation of the carotenoid produced by the genetically modified cells or the carotenoid precursor produced by said genetically modified cells.

The method of the invention enables Blakeslea to be genetically modified in a specific and stable manner, in order to obtain in this way mycelium of cells with uniform nuclei, which produces carotenoids or their precursors, in particular xanthophylls, particularly preferably astaxanthin or zeaxanthin, and phytoene or bixin. The cells are preferably those of fungi of the Blakeslea trispora species. The carotenoids or their precursors produced here are essentially free of contaminations, and it is possible to achieve high concentrations of said carotenoids or their precursors in the culture medium.

Transformation means the transfer of genetic information into the organism, in particular fungus. This should include any possible methods known to the skilled worker of introducing said information, in particular DNA, for example bombardment with DNA-loaded particles, transformation using protoplasts, microinjection of DNA, electroporation, conjugation or transformation of competent cells, chemicals or agrobacteria-mediated transformation. Genetic information means a gene section, a gene or a plurality of genes. The genetic information may be introduced into the cells, for example, with the aid of a vector or as free nucleic acid (e.g. DNA, RNA) and in any other manner, and either be incorporated into the host genome by recombination or be present in a free form in the cell. Particular preference is given here to homologous recombination.

The preferred transformation method is the transformation mediated by Agrobacterium tumefaciens. To this end, the donor DNA to be transferred is first inserted into a vector which (i) carries the T-DNA ends flanking the DNA to be transferred, (ii) includes a selection marker and (iii) has, if appropriate, promoters and terminators for gene expression of the donor DNA. Said vector is transferred into an Agrobacterium tumefaciens strain harboring a Ti plasmid containing the vir genes. vir genes are responsible for DNA transfer in Blakeslea. This two-vector system is used for transferring the DNA from Agrobacterium into Blakeslea. To this end, the Agrobacteria are first incubated in the presence of Acetosyringone. Acetosyringone induces the vir genes. Spores of Blakeslea trispora are then incubated together with the induced cells of Agrobacterium tumefaciens on Acetosyringone-containing medium and thereafter transferred to medium which enables selection of the transformants, i.e. of the genetically modified Blakeslea strains.

The term vector is used in the present application to refer to a DNA molecule which is used for introducing foreign DNA into and, if appropriate, propagating said foreign DNA in a cell (see also “vector” in Römpp Lexikon Chemie—CDROM Version 2.0, Stuttgart/New York: Georg Thieme Verlag 1999). In the present application, the term “vector” is intended to include also plasmids, cosmids etc. which serve the same purpose.

Expression means in the present application the transfer of genetic information, starting from DNA or RNA, to a gene product (here preferably enzymes for producing carotenoids and in particular xanthophylls, particularly preferably astaxanthin or zeaxanthin, and phytoene or bixin), and is also intended to include the term overexpression, meaning increased expression so as for a gene product which is already produced in the untransformed cell (wild type) to be increasingly produced or to form a large part of the entire cell content.

Genetic modification means the introduction of genetic information into a recipient organism so that said information is expressed in a stable manner and passed on during cell division. In this context, homokaryotic conversion is the production of cells which contain only uniform nuclei, i.e. nuclei having the same genetic information content.

This homokaryotic conversion is only required if the genetic information introduced by transformation is recessive, i.e. does not manifest itself. However, if transformation results in the presence of dominant genetic information, i.e. if said information manifests itself, homokaryotic conversion is not absolutely necessary.

The homokaryotic conversion preferably comprises selecting the mononuclear spores. A small proportion of the Blakeslea trispora spores is by nature mononuclear so that these spores can be sorted out, if appropriate after specific labeling, for example staining, of the cell nuclei. This is preferably carried out using FACS (Fluorescence Activated Cell Sorting), on the basis of the lower fluorescence of the mononuclear cells.

Alternatively, the homokaryotic conversion can be carried out by first reducing the number of nuclei. To this end, a mutagenic agent may be employed, in particular N-methyl-N′-nitronitrosoguanidine (MNNG). High energy radiation such as UV radiation or X rays may also be used for reducing the number of nuclei. The subsequent selection may be carried out using the FACS method or recessive selection markers.

Selection means the selection of cells whose nuclei include the same genetic information, i.e. cells which have the same properties such as resistances or production or increased production of a product. Preference is given to using for selection, aside from the FACS method, 5-carbon-5-deazariboflavin (DARF) and hygromycin (hyg) or 5′-fluororotate (FOA) and uracil.

The vector employed in the transformation (i) can be designed so as for the genetic information comprised in said vector to be integrated into the genome of at least one cell. In this connection, genetic information in the cell may be switched off. This may be carried out directly, i.e. by way of a deletion. However, it is also possible for the vector employed in the transformation (i) to be designed in such a way that the genetic information comprised in said vector is expressed in the cell, i.e. genetic information is introduced which is not present in the corresponding wild type or which is increased or overexpressed by said transformation and whose product switches off the gene. The introduced genetic information may, however, switch off genetic information in the cell also indirectly, for example by way of producing an inhibitor.

The vector employed comprises genetic information or parts of said genetic information for producing carotenoids or their precursors, in particular carotenes or xanthophylls or their precursors. The vector employed comprises preferably genetic information for producing astaxanthin, zeaxanthin, echinenone, β-cryptoxanthin, andonixanthin, adonirubine, canthaxanthin, 3-hydroxyechinenone, 3′-hydroxyechinenone, lycopene, lutein, phytofluene, bixin or phytoene. Very particularly preferably, the vector comprises information for producing bixin, phytoene, canthaxanthin, astaxanthin or zeaxanthin.

The vector may comprise any genetic information for genetic modifications of organisms of the Blakeslea genus.

“Genetic information” means preferably nucleic acids whose introduction into the organism of the Blakeslea genus results in a genetic modification in organisms of the Blakeslea genus, i.e., for example, in causing, increasing or reducing enzyme activities in comparison with the starting organism.

The vector may comprise, for example, genetic information for producing lipophilic substances such as, for example, carotenoids and their precursors, phospholipids, triacylglycerides, steroids, waxes, fat-soluble vitamins, provitamins and cofactors or genetic information for producing hydrophilic substances such as, for example, proteins, amino acids, nucleotides and water-soluble vitamins, provitamins and cofactors.

The vector employed preferably comprises genetic information for producing carotenoids or xanthophylls or their precursors.

The vector preferably comprises genetic information causing the carotenoid biosynthesis enzymes to be located in the cell compartment in which carotenoid biosynthesis takes place.

Particular preference is given to genetic information for producing astaxanthin, zeaxanthin, echinenone, β-cryptoxanthin, andonixanthin, adonirubin, canthaxanthin, 3- and 3′-hydroxyechinenone, lycopene, lutein, β-carotene, phytoene and/or phytofluene. Very particular preference is given to genetic information for producing phytoene, bixin, lycopene, zeaxanthin, canthaxanthin and/or astaxanthin.

Accordingly, a preferred variant of the invention comprises producing and culturing organisms having an increased rate of synthesis of carotenoid biosynthesis intermediates and consequently increased productivity for final products of carotenoid biosynthesis. The rate of synthesis of carotenoid biosynthesis intermediates is increased in particular by increasing the activities of the enzymes 3-hydroxy-3-methylglutaryl coenzyme A reductase (HMG-CoA reductase), isopentenyl pyrophosphate isomerase and geranyl pyrophosphate synthase.

Accordingly, a particularly preferred variant of the invention comprises producing and culturing organisms having an increased HMG-CoA reductase activity compared to the wild type.

HMG-CoA reductase activity means the enzyme activity of an HMG-CoA reductase (3-hydroxy-3-methylglutaryl coenzyme A reductase).

HMG-CoA reductase means a protein which has the enzymic activity of converting 3-hydroxy-3-methylglutaryl coenzyme A to mevalonate.

Accordingly, HMG-CoA reductase activity means the amount of 3-hdroxy-3-methylglutaryl-coenzyme A converted or the amount of mevalonate produced by the protein HMG-CoA reductase within a particular time.

In the case of increased HMG-CoA reductase activity compared with the wild type, thus the protein HMG-CoA reductase increases the amount of 3-hydroxy-3-methylglutaryl coenzyme A converted or the amount of mevalonate produced within a particular time in comparison with the wild type.

This increase in HMG-CoA reductase activity is preferably at least 5%, more preferably at least 20%, more preferably at least 50%, more preferably at least 100%, particularly preferably at least 300%, still more preferably at least 500%, in particular at least 600%, of the HMG-CoA reductase activity of the wild type.

In a preferred embodiment, the HMG-CoA reductase activity is increased compared to the wild type by increasing gene expression of a nucleic acid encoding an HMG-CoA reductase.

In a particularly preferred embodiment of the method of the invention, gene expression of a nucleic acid encoding an HMG-CoA reductase is increased by introducing into the organism a nucleic acid construct comprising a nucleic acid encoding an HMG-CoA reductase whose expression in said organism is subject to a reduced regulation, compared with the wild type.

Reduced regulation compared with the wild type means a reduced, preferably no, regulation at the expression or protein level in comparison with the wild type defined above.

Reduced regulation may preferably also be achieved by a promoter which is functionally linked to the coding sequence in the nucleic acid construct and which is subject to a reduced regulation in the organism, compared with the wild type promoter.

For example, the promoters ptef1 of Blakeslea trispora and pgpdA of Aspergillus nidulans are subject only to reduced regulation and are therefore particularly preferred promoters.

These promoters exhibit nearly constitutive expression in Blakeslea trispora so that transcriptional regulation no longer takes place via the intermediates of carotenoid biosynthesis.

In a further preferred embodiment, said reduced regulation can be achieved by using a nucleic acid encoding an HMG-CoA reductase, whose expression in said organism is subject to a reduced regulation, compared with the orthologous nucleic acid intrinsic to said organism.

Particular preference is given to using a nucleic acid which encodes only the catalytic region of HMG-CoA reductase (truncated (t-)HMG-CoA reductase). The membrane domain responsible for regulation is absent. The nucleic acid used is thus subject to reduced regulation and thus results in an increase of gene expression of HMG-CoA reductase.

In a particularly preferred embodiment, nucleic acids comprising the sequence SEQ ID. NO. 75 are introduced into Blakeslea trispora.

Further examples of HMG-CoA reductases and thus also of the t-HMG-CoA reductases reduced to the catalytic region or the encoding genes can readily be found, for example, from various organisms whose genomic sequence is known by homology comparisons of the sequences from databases with SEQ ID. NO. 75.

Further examples of HMG-CoA reductases and thus also of the t-HMG-CoA reductases reduced to the catalytic region or the encoding genes can furthermore readily be found, for example starting from the sequence SEQ ID. NO. 75, from various organisms whose genomic sequence is not known, by hybridization and PCR techniques in a manner known per se.

In a particularly preferred embodiment, said reduced regulation is achieved by using a nucleic acid encoding an HMG-CoA reductase, whose expression in said organism is subject to a reduced regulation, compared with the orthologous nucleic acid intrinsic to said organism, and using a promoter which is subject to a reduced regulation in said organism, compared with the wild type promoter.

Accordingly, a preferred variant of the invention comprises the transformation switching off phytoene desaturase gene expression, thus enabling the phytoene produced by the organisms to be isolated. The vector employed in the transformation (i) therefore comprises in one embodiment of the invention preferably a sequence coding for a fragment of the gene of phytoene desaturase, in particular Blakeslea trispora carB, with SEQ ID NO: 69.

Accordingly, a preferred variant of the invention comprises lycopene cyclase gene expression being switched off by transformation, thus enabling the lycopene produced by the organisms to be isolated. The vector employed in said transformation therefore comprises in one embodiment of the invention preferably a sequence coding for a fragment of the lycopene cyclase gene, in particular Blakeslea trispora carR.

In a preferred embodiment, the organisms of the Blakeslea genus are enabled, for example, to produce xanthophylls such as, for example, canthaxanthin, zeaxanthin or astaxanthin, bixin or phytoene by causing a hydroxylase activity and/or ketolase activity in the genetically modified organisms of the Blakeslea genus, in comparison with the wild type.

Thus, in a further, preferred variant of the invention, the vector employed in the transformation (i) comprises genetic information which, after expression, displays a ketolase and/or hydroxylase activity so that the organisms produce zeaxanthin or astaxanthin.

Ketolase activity means the enzyme activity of a ketolase.

A ketolase means a protein which has the enzymic activity of introducing a keto group at the optionally substituted β-ionone ring of carotenoids.

A ketolase means in particular a protein which has the enzymic activity of converting β-carotene to canthaxanthin.

Accordingly, ketolase activity means the amount of β-carotene converted or the amount of canthaxanthin produced by the protein ketolase within a particular time.

According to the invention, the term “wild type” means the corresponding genetically unmodified starting organism of the Blakeslea genus.

The term “organism” may mean the starting organism (wild type) of the Blakeslea genus or a genetically modified organism according to the invention of the Blakeslea genus or both, depending on the context.

Preferably “wild type” for causing the ketolase activity and for causing the hydroxylase activity means in each case a reference organism.

This reference organism of the Blakeslea genus is Blakeslea trispora ATCC 14271 or ATCC 14272 which differ merely with respect to the mating type.

The ketolase activity in genetically modified organisms according to the invention of the Blakeslea genus and in wild type or reference organisms is preferably determined under the following conditions:

The ketolase activity in organisms of the Blakeslea genus is determined following the method of Frazer et al., (J. Biol. Chem. 272(10): 6128-6135, 1997). The ketolase activity in extracts is determined using the substrates beta-carotene and canthaxanthin in the presence of lipid (soya lecithin) and detergent (sodium cholate). Substrate-to-product ratios of the ketolase assays are determined by means of HPLC.

In this preferred embodiment, the genetically modified organism according to the invention of the Blakeslea genus has, in comparison with the genetically unmodified wild type, a ketolase activity and is thus preferably capable of transgenically expressing a ketolase.

In a further preferred embodiment, the ketolase activity in the organisms of the Blakeslea genus is caused by causing gene expression of a nucleic acid encoding a ketolase.

In this preferred embodiment, gene expression of a nucleic acid encoding a ketolase is preferably caused by introducing nucleic acids encoding ketolases into the starting organism of the Blakeslea genus.

For this purpose, it is possible in principle to use any ketolase gene, i.e. any nucleic acid encoding a ketolase.

Any of the nucleic acids mentioned in the description may be an RNA, DNA or cDNA sequence for example.

In the case of genomic ketolase sequences from eukaryotic sources, which include introns, preference is given to using already processed nucleic acid sequences such as the corresponding cDNAs, if the host organism of the Blakeslea genus is unable or cannot be made to express the corresponding ketolase.

Examples of nucleic acids encoding a ketolase and the corresponding ketolases, which may be used in the method of the invention, are, for example, sequences from:

Haematoccus pluvialis, in particular from Haematoccus pluvialis Flotow em. Wille (accession NO: X86782; nucleic acid: SEQ ID NO: 11, protein SEQ ID NO: 12),

Haematoccus pluvialis, NIES-144 (accession NO: D45881; nucleic acid: SEQ ID NO: 13, protein SEQ ID NO: 14),

Agrobacterium aurantiacum (accession NO: D58420; nucleic acid: SEQ ID NO: 15, protein SEQ ID NO: 16),

Alicaligenes spec. (accession NO: D58422; nucleic acid: SEQ ID NO: 17, protein SEQ ID NO: 18),

Paracoccus marcusii (accession NO: Y15112; nucleic acid: SEQ ID NO: 19, protein SEQ ID NO: 20).

Synechocystis sp. Strain PC6803 (accession NO: NP442491; nucleic acid: SEQ ID NO: 21, protein SEQ ID NO: 22).

Bradyrhizobium sp. (accession NO: AF218415; nucleic acid: SEQ ID NO: 23, protein SEQ ID NO: 24).

Nostoc sp. Strain PCC7120 (accession NO: AP003592, BAB74888; nucleic acid: SEQ ID NO: 25, protein SEQ ID NO: 26),

Nostoc punctiforme ATTC 29133, Nucleic acid: Acc. No. NZ_AABC01000195, base pair 55,604 to 55,392 (SEQ ID NO: 27); Protein: Acc. No. ZP_{—}00111258 (SEQ ID NO: 28) (annotated as putative protein),

Nostoc punctiforme ATTC 29133, Nucleic acid: Acc. No. NZ_AABC01000196, base pair 140,571 to 139,810 (SEQ ID NO: 29), protein: (SEQ ID NO: 30) (not annotated),

Further natural examples of ketolases and ketolase genes, which may be used in the process of the invention, can be readily found, for example, from various organisms whose genomic sequence is known by comparing the identities of the amino acid sequences or of the corresponding back translated nucleic acid sequences from databases with those of the previously described sequences and in particular with those of the sequences SEQ ID NO: 12, 26 and/or 33.

Further natural examples of ketolases and ketolase genes can furthermore be readily found, starting from the previously described nucleic acid sequences, in particular starting from the sequences SEQ ID NO: 12, 26 and/or 30, from various organisms whose genomic sequence is not known, using hybridization techniques in a manner known per se.

The hybridization may be carried out under moderate (low stringency) or, preferably, under stringent (high stringency) conditions.

Hybridization conditions of these types are described, for example, in Sambrook, J., Fritsch, E. F., Maniatis, T., in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6.

For example, the conditions during the washing step may be selected from the range of conditions limited by those of low stringency (with 2×SSC at 50° C.) and those of high stringency (with 0.2×SSC at 50° C., preferably at 65° C.) (20×SSC: 0.3 M sodium citrate, 3 M sodium chloride, pH 7.0).

An additional possibility is to rise the temperature during the washing step from moderate conditions at room temperature, 22° C., up to stringent conditions at 65° C.

Both parameters, the salt concentration and temperature, can be varied simultaneously, and it is also possible to keep one of the two parameters constant and vary only the other one. It is also possible to employ denaturing agents such as, for example, formamide or SDS during the hybridization. Hybridization in the presence of 50% formamide is preferably carried out at 42° C.

Some examples of conditions for hybridization and washing step are given below:

(1) hybridization conditions with, for example,

(i) 4×SSC at 65° C., or

(ii) 6×SSC at 45° C., or

(iii) 6×SSC at 68° C., 100 mg/ml denatured fish sperm DNA, or

(iv) 6×SSC, 0.5% SDS, 100 mg/ml denatured, fragmented salmon sperm DNA at 68° C., or

(v) 6×SSC, 0.5% SDS, 100 mg/ml denatured, fragmented salmon sperm DNA, 50% formamide at 42° C., or

(vi) 50% formamide, 4×SSC at 42° C., or (vii) 50% (vol/vol) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C., or

(viii) 2× or 4×SSC at 50° C. (moderate conditions), or

(ix) 30 to 40% formamide, 2× or 4×SSC at 42° C. (moderate conditions).

(2) Washing steps of 10 minutes each with, for example,

(i) 0.015 M NaCl/0.0015 M sodium citrate/0.1% SDS at 50° C., or

(ii) 0.1×SSC at 65° C., or

(iii) 0.1×SSC, 0.5% SDS at 68° C., or

(iv) 0.1×SSC, 0.5% SDS, 50% formamide at 42° C., or

(v) 0.2×SSC, 0.1% SDS at 42° C., or

(vi) 2×SSC at 65° C. (moderate conditions).

In a preferred embodiment of the genetically modified organisms according to the invention of the Blakeslea genus, nucleic acids are introduced which encode a protein comprising the amino acid sequence SEQ ID NO: 12 or a sequence which is derived from this sequence by substitution, insertion or deletion of amino acids and which has an identity of at least 20%, preferentially at least 30%, 40%, 50%, 60%, preferably at least 70%, 80%, particularly preferably at least 90%, in particular 91%, 92%; 93%, 94%, 95%, 96%, 97%, 98% or 99%, at the amino acid level with the sequence SEQ ID NO: 12 and which has the enzymic property of a ketolase.

In this connection, it is possible for the ketolase sequence to be a natural one which can be found as described above by identity comparison of the sequences from other organisms, or for the ketolase sequence to be an artificial one which has been modified starting from the sequence SEQ ID NO: 12 by artificial variation, for example by substitution, insertion or deletion of amino acids.

A further, preferred embodiment of the methods of the invention involves introducing nucleic acids which encode a protein comprising the amino acid sequence SEQ ID NO: 26 or a sequence which is derived from this sequence by substitution, insertion or deletion of amino acids and which has an identity of at least 20%, preferentially at least 30%, 40%, 50%, 60%, preferably at least 70%, 80%, particularly preferably at least 90%, in particular 91%, 92%; 93%, 94%, 95%, 96%, 97%, 98% or 99%, at the amino acid level with the sequence SEQ ID NO: 26 and which has the enzymic property of a ketolase.

In this connection, it is possible for the ketolase sequence to be a natural one which can be found as described above by identity comparison of the sequences from other organisms, or for the ketolase sequence to be an artificial one which has been modified starting from the sequence SEQ ID NO: 26 by artificial variation, for example by substitution, insertion or deletion of amino acids.

A further, preferred embodiment of the methods of the invention involves introducing nucleic acids which encode a protein comprising the amino acid sequence SEQ ID NO: 30 or a sequence which is derived from this sequence by substitution, insertion or deletion of amino acids and which has an identity of at least 20%, preferentially at least 30%, 40%, 50%, preferably at least 60%, 70%, more preferably at least 80%, 85%, particularly preferably at least 90%, in particular 91%, 92%, 93%, 94%, 95%, 96%, 97%, 98% or 99%, at the amino acid level with the sequence SEQ ID NO: 30 and which has the enzymic property of a ketolase.

In this connection, it is possible for the ketolase sequence to be a natural one which can be found as described above by identity comparison of the sequences from other organisms, or for the ketolase sequence to be an artificial one which has been modified starting from the sequence SEQ ID NO: 30 by artificial variation, for example by substitution, insertion or deletion of amino acids.

The term “substitution” means in the description substitution of one or more amino acids by one or more amino acids. Preference is given to carrying out “conservative” substitutions in which the replaced amino acid has a similar property to the original amino acid, for example substitution of Glu by Asp, Gln by Asn, Val by Ile, Leu by Ile, Ser by Thr.

Deletion is the replacement of an amino acid by a direct bond. Preferred positions for deletions are the termini of the polypeptide and the linkages between the individual protein domains.

Insertions are insertions of amino acids into the polypeptide chain, with formal replacement of a direct bond by one or more amino acids.

Identity between two proteins means the identity of the amino acids over the entire length of each protein, in particular the identity calculated by comparison with the aid of Lasergene software from DNASTAR, inc. Madison, Wis. (USA) using the Clustal method (Higgins D G, Sharp P M. Fast and sensitive multiple sequence alignments on a microcomputer. Comput Appl. Biosci. 1989 April; 5(2):151-1), setting the following parameters:

Multiple Alignment Parameter:

| Multiple alignment parameter: | |
|---|---|
| Gap penalty | 10 |
| Gap length penalty | 10 |
| Pairwise alignment parameter: | |
| K-tuple | 1 |
| Gap penalty | 3 |
| Window | 5 |
| Diagonals saved | 5 |

Accordingly, a protein which has an identity of at least 20% at the amino acid level with the sequence SEQ ID NO: 12 or 26 or 30 means a protein which, on comparison of its sequence with the sequence SEQ ID NO: 12 or 26 or 30, in particular using the above program logarithm with the above set of parameters, has an identity of at least 20%, preferably 30%, 40%, 50%, particularly preferably 60%, 70%, 80%, in particular 85%, 90, 95%.

Suitable nucleic acid sequences can be obtained, for example, by back translation of the polypeptide sequence in accordance with the genetic code.

The codons preferably used for this purpose are those frequently used according to the Blakeslea-specific codon usage. The codon usage can easily be found by means of computer analyses of other, known genes of organisms of the Blakeslea genus.

In a particularly preferred embodiment, a nucleic acid comprising the sequence SEQ ID NO: 11 is introduced into the organism of said genus.

In a particularly preferred embodiment, a nucleic acid comprising the sequence SEQ ID NO: 25 is introduced into the organism of said genus.

In a particularly preferred embodiment, a nucleic acid comprising the sequence SEQ ID NO: 29 is introduced into the organism of said genus.

All the aforementioned ketolase genes can moreover be prepared in a manner known per se by chemical synthesis from the nucleotide building blocks, for example by fragment condensation of individual overlapping, complementary nucleic acid building blocks of the double helix. Chemical synthesis of oligonucleotides is possible, for example, in a known manner by the phosphoamidite method (Voet, Voet, 2nd edition, Wiley Press New York, pages 896-897). Addition of synthetic oligonucleotides and filling in of gaps with the aid of the Klenow fragment of DNA polymerase and ligation reactions, and also general cloning methods are described in Sambrook et al. (1989), Molecular cloning: A laboratory manual, Cold Spring Harbor Laboratory Press.

The vector employed in the transformation (i) therefore comprises in one embodiment of the invention preferably a sequence coding for a ketolase, in particular the Nostoc punctiforme ketolase with SEQ ID NO: 72.

Hydroxylase activity means the enzymic activity of a hydroxylase.

A hydroxylase means a protein having the enzymic activity of introducing a hydroxyl group on the, optionally substituted, β-ionone ring of carotenoids.

In particular, a hydroxylase means a protein having the enzymic activity of converting β-carotene to zeaxanthin or cantaxanthin to astaxanthin.

Accordingly, hydroxylase activity means the amount of β-carotene or cantaxanthin converted, or amount of zeaxanthin or astaxanthin produced, by the hydroxylase protein in a particular time.

Thus, when the hydroxylase activity is increased compared with the wild type, the amount of β-carotene or cantaxantin converted or the amount of zeaxanthin or astaxanthin produced in a particular time by the hydroxylase protein is increased in comparison with the wild type.

This increase in hydroxylase activity is preferably at least 5%, further preferably at least 20%, further preferably at least 50%, further preferably at least 100%, more preferably at least 300%, still more preferably at least 500%, in particular at least 600%, of the hydroxylase activity of the wild type.

The hydroxylase activity in the genetically modified organisms of the invention and in wild-type and reference organisms is preferably determined under the following conditions:

The hydroxylase activity is determined by the method of Bouvier et al. (Biochim. Biophys. Acta 1391 (1998), 320-328) in vitro. Ferredoxin, Ferredoxin-NADP oxidoreductase, katalase, NADPH and beta-carotene are added with mono- and digalactosyl glycerides to a defined amount of organism extract.

The hydroxylase activity is particularly preferably determined under the following conditions of Bouvier, Keller, d'Harlingue and Camara (Xanthophyll biosynthesis: molecular and functional characterization of carotenoid hydroxylases from pepper fruits (Capsicum annuum L.; Biochim. Biophys. Acta 1391 (1998), 320-328):

The in vitro assay is carried out in a volume of 0.250 ml. The mixture contains 50 mM potassium phosphate (pH 7.6), 0.025 mg of spinach ferredoxin, 0.5 unit of spinach ferredoxin-NADP+ oxidoreductase, 0.25 mM NADPH, 0.010 mg of beta-carotene (emulsified in 0.1 mg of Tween 80), 0.05 mM of a mixture of mono- and digalactosyl glycerides (1:1), 1 unit of catalysis, 200 mono- and digalactosyl glycerides, (1:1), 0.2 mg of bovine serum albumin and organism extract in a varying volume. The reaction mixture is incubated at 30° C. for 2 hours. The reaction products are extracted with an organic solvent such as acetone or chloroform/methanol (2:1) and determined by HPLC.

The hydroxylase activity is particularly preferably determined under the following conditions of Bouvier, d'Harlingue and Camara (Molecular Analysis of carotenoid cyclae inhibition; Arch. Biochem. Biophys. 346(1) (1997) 53-64):

The in vitro assay is carried out in a volume of 250 μl. The mixture contains 50 mM potassium phosphate (pH 7.6), varying amounts of organism extract, 20 nM lycopene, 250 μg of paprika chromoplastid stromal protein, 0.2 mM NADP+, 0.2 mM NADPH and 1 mM ATP. NADP/NADPH and ATP are dissolved in 10 ml of ethanol with 1 mg of Tween 80 immediately before addition to the incubation medium. After a reaction time of 60 minutes at 30° C., the reaction is stopped by adding chloroform/methanol (2:1). The reaction products extracted into chloroform are analyzed by HPLC.

An alternative assay with radioactive substrate is described in Fraser and Sandmann (Biochem. Biophys. Res. Comm. 185(1) (1992) 9-15).

The hydroxylase activity can be increased in various ways, for example by switching off inhibitory regulatory mechanisms at the expression and protein levels or by increasing gene expression of nucleic acids encoding a hydroxylase, compared with the wild type.

Gene expression of the nucleic acids encoding a hydroxylase can likewise be increased, compared with the wild type, in various ways, for example by inducing the hydroxylase gene by activators or by introducing one or more hydroxylase gene copies, i.e. by introducing at least one nucleic acid encoding a hydroxylase into the organism of the Blakeslea genus.

In a preferred embodiment, gene expression of a nucleic acid encoding a hydroxylase is increased by introducing at least one nucleic acid encoding a hydroxylase into the organism of the Blakeslea genus.

It is possible to use for this purpose in principle any hydroxylase gene, i.e. any nucleic acid which encodes a hydroxylase and any nucleic acid which encodes a cyclase.

In the case of genomic hydroxylase sequences from eukaryotic sources, which comprise introns, preference is given to using nucleic acid sequences which have already been processed, such as the corresponding cDNAs, if the host organism is unable or cannot be made to express the corresponding hydroxylase.

One example of a hydroxylase gene is a nucleic acid encoding a Haematococcus pluvialis hydroxylase, with accession No. AX038729 (WO 0061764; nucleic acid: SEQ ID NO: 31, protein: SEQ ID NO: 32), an Erwinia uredovora 20D3 hydroxylase (ATCC 19321, accession No. D90087; nucleic acid: SEQ ID NO: 33, protein: SEQ ID NO: 34) or Thermus thermophilus hydroxylase (DE 102 34 126.5) encoded by the sequence SEQ ID NO 76.

and also hydroxylases of the following accession numbers: |emb|CAB55626.1, CAA70427.1, CAA70888.1, CAB55625.1, AF499108_{—}1, AF315289_{—}1, AF296158_{—}1, AAC49443.1, NP_{—}194300.1, NP_{—}200070.1, AAG10430.1, CAC06712.1, AAM88619.1, CAC95130.1, AAL80006.1, AF162276_{—}1, AA053295.1, AAN85601.1, CRTZ_ERWHE, CRTZ_PANAN, BAB79605.1, CRTZ_ALCSP, CRTZ_AGRAU, CAB56060.1, ZP_{—}00094836.1, AAC44852.1, BAC77670.1, NP_{—}745389.1, NP_{—}344225.1, NP_{—}849490.1, ZP_{—}00087019.1, NP_{—}503072.1, NP_{—}852012.1, NP_{—}115929.1, ZP_{—}00013255.1

Thus, in this preferred embodiment, at least one further hydroxylase gene is present in the preferred transgenic organisms according to the invention of the Blakeslea genus, compared with the wild type.

In this preferred embodiment, the genetically modified organism has, for example, at least one exogenous nucleic acid encoding a hydroxylase or at least two endogenous nucleic acids encoding a hydroxylase.

In the preferred embodiment described above, preference is given to using as hydroxylase genes nucleic acids which encode proteins comprising the amino acid sequence SEQ ID NO: 32, 34 or encoded by the sequence SEQ ID NO 76 or a sequence which is derived from this sequence by substitution, insertion or deletion of amino acids and which has an identity of least 30%, preferably at least 50%, more preferably at least 70%, still more preferably at least 80%, more preferably at least 90%, in particular 91%, 92%, 93%, 94%, 95%, 96%, 97%, 98%, 99%, at the amino acid level to the sequence SEQ. ID. NO: 32, 34, or encoded by the sequence with SEQ ID NO 76, and which have the enzymic property of a hydroxylase.

Further examples of hydroxylases and hydroxylase genes can readily be found, for example, from various organisms whose genomic sequence is known, as described above, by homology comparisons of the amino acid sequences or of the corresponding back-translated nucleic acid sequences from databases with SEQ ID. NO: 31, 33 or 76.

Further examples of hydroxylases and hydroxylase genes can furthermore readily be found in a manner known per se, for example starting from the sequence SEQ ID NO: 31, 33 or 76, from various organisms whose genomic sequence is unknown, as described above, by hybridization and PCR techniques.

In a further particularly preferred embodiment, nucleic acids which encode proteins comprising the amino acid sequence of the hydroxylase of sequence SEQ ID NO: 32, 34 or encoded by the sequence SEQ ID NO 76 are introduced into organisms to increase the hydroxylase activity.

Suitable nucleic acid sequences can be obtained, for example, by back translation of the polypeptide sequence in accordance with the genetic code.

Preference is given to using for this purpose those codons which are frequently used in accordance with the organism-specific codon usage. The codon usage can readily be determined on the basis of computer analyses of other, known genes of the organisms in question.

In a particularly preferred embodiment, a nucleic acid comprising the sequence SEQ. ID. NO: 31, 33 or 76 is introduced into the organism.

All the aforementioned hydroxylase genes can furthermore be prepared in a manner known per se by chemical synthesis from the nucleotide building blocks, for example by fragment condensation of individual overlapping, complementary nucleic acid building blocks of the double helix. Chemical synthesis of oligonucleotides is possible, for example, in a known manner by the phosphoamidite method (Voet, Voet, 2nd edition, Wiley Press New York, pages 896-897). Addition of synthetic oligonucleotides and filling in of gaps with the aid of the Klenow fragment of DNA polymerase and ligation reactions, and also general cloning methods are described in Sambrook et al. (1989), Molecular cloning: A laboratory manual, Cold Spring Harbor Laboratory Press.

The vector employed in the transformation (i) therefore comprises in a further embodiment of the invention preferably a sequence coding for a hydroxylase, in particular a Haematococcus pluvialis hydroxylase with SEQ ID NO: 70 or an Erwinia uredova hydroxylase with SEQ ID NO: 71 or a Thermus thermophilus hydroxylase encoded by the sequence SEQ ID NO 76.

Preference is given to the transformation switching off the gene of phytoene desaturase.

The vector employed in the transformation (i) preferably also includes regions which control and support expression, in particular promoters and terminators.

The vector employed in the transformation (i) preferably includes the gpd and/or the ptef1 promoter and/or the trpc terminator, all of which have proved to be particularly successful in the transformation of Blakeslea. The use of “inverted repeats” familiar to the skilled worker (IR, Römpp Lexikon der Biotechnologie 1992, Thieme Verlag Stuttgart, page 407 “Inverse repetitive sequences”) for controlling expression and transcription is also within the scope of the invention.

The gpd promoter employed in the vector has advantageously the sequence SEQ ID NO: 1. The trpc terminator employed in the vector has advantageously the sequence SEQ ID NO: 2. The ptef1 promoter employed in the vector has advantageously the sequence SEQ ID NO: 35.

Preference is given here to using in particular the gpd promoter and the trpC terminator from Aspergillus nidulans and the ptef1 promoter from Blakeslea trispora.

The vector employed in the transformation (i) in particular comprises a resistance gene. The latter is preferably a hygromycin resistance gene (hph), in particular one from E. coli. This resistance gene has proved particularly suitable in the detection of transformation and selection of the cells.

The preferred promoter utilized for hph thus is p-gpdA, the promoter of glyceraldehyde 3-phosphate dehydrogenase coding for Aspergillus nidulans. The preferred terminator utilized for hph is t-trpC, the terminator of the trpC gene coding for Aspergillus nidulans anthranilate synthase components.

Derivatives of the pBinAHyg vector have proved to be particularly suitable vectors. The vector employed for transformation thus preferably comprises SEQ ID NO: 3. To this will be added, depending on the desired carotenoid or its precursor, a sequence coding for a hydroxylase, ketolase, phytoene desaturase etc., as described above. The vectors thus comprise in one embodiment of the invention the sequence SEQ ID NO: 69 coding for said phytoene desaturase. The vectors also comprise in a further embodiment of the invention the sequence SEQ ID NO: 72 coding for a ketolase. The vectors further comprise in a further embodiment of the invention the sequence SEQ ID NO: 70 or 71 or 76 coding for a hydroxylase. Corresponding combinations of the abovementioned sequences are also within the scope of the invention. Thus, the vector comprises in one embodiment both a sequence SEQ ID NO: 72 coding for a ketolase and the sequence SEQ ID NO: 70 or 71 or 76 coding for a hydoxylase and thus enables astaxanthin to be produced.

In particular, it is possible to use within the scope of the invention vectors selected from the group consisting of SEQ ID NO: 37 to 51 and 62.

The genetically modified organisms may be used for producing carotenoids, xanthophylls or their precursors, in particular bixin, phytoene, astaxanthin, zeaxanthin and canthaxanthin. It is also possible, by introducing the appropriate genetic information, for new carotenoids which do not occur naturally in the wild type to be generated by the specifically genetically modified cells or by the mycelium formed thereby and subsequently to be isolated.

After selection, the genetically modified cells are cultured in order to be able to provide carotenoids or their precursors.

Preference is given to obtaining carotenoids or their precursors using the specifically genetically modified cells or the mycelium formed thereby.

The cultivation of the organisms has no special requirements. Advantageously, in particular when using Blakeslea trispora, opposite mating types are cultured together, since this results in better growth and production.

If the genetic modification is carried out only in cells of one of the mating types found ((+) or (−) for Blakeslea trispora), the corresponding other, unmodified mating type is added to the cultivation, since it is possible in this way to achieve good production of the carotenoids or their precursors, owing to the substances released by the second, unmodified mating type (e.g. trisporic acids). Advantageously, however, the genetic modification is carried out in cells of both mating types which are then cultured together, thereby achieving particularly good growth and optimal production of the carotenoids or their precursors. An (artificial) addition of trisporic acids is possible and useful.

Trisporic acids are sex hormones in Mucorales fungi such as Blakeslea, which stimulate the formation of zygophores and production of β-carotene (van den Ende 1968, J. Bacteriol. 96:1298-1303, Austin et al. 1969, Nature 223:1178-1179, Reschke Tetrahedron Lett. 29:3435-3439, van den Ende 1970, J. Bacteriol. 101:423-428).

It is possible to use any media familiar to the skilled worker, as long as they are suitable for culturing the organisms used and carotenoid production thereof. In particular, the use of carotenoid biosynthesis inhibitors is not necessary when the “GMO” (genetically modified organisms) are used. The media employed preferably include additives such as one or more carbon sources, one or more nitrogen sources, mineral salts and thiamine. Preference is given to employing additives as disclosed in WO 03/038064 A2, page 4, line 30 to page 5 line 7. A particularly preferred carbon source is glucose and particularly preferred nitrogen sources are asparagine, plant or animal extracts such as cotton seed oil, soybean oil, cotton seed meal or yeast extract.

The cultivation may be carried out either under aerobic or anaerobic conditions. A mixed, first aerobic and then anaerobic, cultivation, as disclosed in DE 101 30 323, is also possible. In this case, temperature and humidity are set in each case for optimal growth. The temperature of the cultivation is preferably between approx. 20 and approx. 34° C., in particular between approx. 26° C. and approx. 28° C. Furthermore, the cultivation may be carried out continuously or batchwise.

The cultivation is preferably carried out up to a solids content between about 1 and about 20%, preferably 3 and 15% and particularly preferably 4 and 11%. Particularly important is the fact that the culture broth remains pumpable so as to remain processible in the subsequent process steps. If the solids content is too low, complicated concentration or drying steps are required.

The cultivation or fermentation may be carried out in the usual apparatus. This includes all apparatus suitable for the microorganisms employed in each case and their products, in particular those indicated under the keyword “bioreactor” on pages 123-126 of Römpp Lexikon Biotechnologie (1992 Georg Thieme Verlag, Stuttgart). Particular preference is given to using stirred tank reactors with various internal fittings, various types of bubble columns, etc.

The carotenoids or their precursors provided by the method of the invention, in particular bixin, phytoene or xanthophylls, particularly preferably astaxanthin or zeaxanthin, are particularly suitable for producing additives for feedstuffs, foodstuffs and food supplements, cosmetic, pharmaceutical or dermatological preparations.

The carotenoid produced by the genetically modified cells or the carotenoid precursor produced by the genetically modified cells is prepared from the culture of the genetically modified microorganisms according to two variants, a) or b), with preference also being given to a combination of a) and b);

a:

I) removal of the biomass,
   IA) optional washing of the biomass with a solvent in which carotenoids are not soluble, in particular water,
   IB) sterilization and cell disruption of the biomass,
   IC) optional drying and/or homogeneous distribution, and
II) partial extraction of the carotenoids from the disrupted biomass by means of a carotenoid-dissolving solvent and separation of said solvent from said biomass,
   IIA)
   1) removal of residual solvent from the carotenoid-containing biomass,
   2) optional homogeneous suspension of the biomass, with a biomass solid content of >2% and <50%, and
   3) drying of the biomass or suspension for producing the foodstuff,
   IIB)
   1) crystallization of the carotenoids from the solvent used and isolation of the carotenoid crystals, in particular by filtration;
      or b):
I) homogeneous suspension of the solids of the culture broth, and
IIA) for a solid content of the culture broth of >2%:
   1) optional concentration of the culture broth to give a solid content of <50%, and
   2) drying of the culture broth to produce the foodstuff, or
IIB) for a solid content of <2% of the culture broth,
   1) concentration of the culture broth to give a solid content of >2% and <50%, and
   2) drying of the suspension to produce the foodstuff, or
IIC) independently of the solid content of the culture broth,
   1) removal of the biomass,
   2) optional washing of the biomass with solvents in which carotenoids are not soluble, in particular water,
   3) sterilization and cell disruption,
   4) optional drying and homogeneous distribution,
   5) partial extraction of the carotenoids from the biomass using a carotenoid-dissolving solvent,
   5a) removal of the carotenoid-containing biomass from the carotenoid-containing solvent,
   5b) removal of residual solvent from the biomass, and
   5c) drying of the biomass to produce the foodstuff,
   6) crystallization of the carotenoids from the solvent used in 5a) and isolation of the carotenoid crystals, in particular by filtration.

The preparation according to the invention of the carotenoid produced by the genetically modified cells or of the carotenoid precursor produced by the genetically modified cells from the culture of the genetically modified microorganisms, carried out according to two variants a) or b), enables two products to be produced at the same time.

By combining according to the invention the production of two products, in particular in the preparation according to variant a), namely the at least one carotenoid and the carotenoid-containing foodstuff, there is no need for complete extraction of the carotenoids from the biomass so that said extraction is less complicated. Despite complete utilization, the carotenoid needs to be extracted only partially, and no product is lost. This requires small amounts of solvent, accompanied by fewer measures for their reuse. Moreover, waste products are largely avoided, since the biomass does not end up as waste but is processed further to give a foodstuff of high value. As a result, the methods become less expensive due to the utilization of synergies.

Thus, foodstuffs obtainable by the method according to the invention with preparation according to variant b) include already after production large amounts of carotenoids which need not be added. Moreover, the nutrient content of said foodstuff is increased, due to the fact that it also contains Blakeslea trispora, in addition to the at least one carotenoid. The increase in nutrient content is particularly large according to the preferred alternatives IIA and IIB, since it includes, aside from the at least one carotenoid and Blakeslea trispora, in addition all media components of the fermentation. Furthermore, the process does not require any additional, complex work-up and preparation steps; rather, the homogenized and, if appropriate, dehydrated culture broth containing Blakeslea trispora can be dried directly to produce the foodstuff. As a result, there are virtually no waste products, apart from the aqueous medium in alternative IIB, which, however, can be purified without problems in a purification plant. In addition, all three alternatives utilize the entire amount of carotenoids produced without or with only marginal losses, since, according to IIA and IIB, no separation or work-up steps with heavy losses need to be carried out. In alternative IIIC, the entire amount of carotenoids produced is likewise utilized without or with only marginal losses, since one part is processed within the biomass to give the foodstuff and the other part is extracted to obtain pure carotenoids. The combination according to the invention of producing two products according to IIC, namely the carotenoid-containing foodstuff and the carotenoids per se, results in the advantage that again essentially no waste products occur and complete extraction of the carotenoids from the biomass is unnecessary so that the usually complex extraction is less complex. Despite complete utilization, the valuable carotenoid(s) need to be extracted only partially, without incurring product losses.

This requires small amounts of solvent, accompanied by fewer measures for their reuse. Moreover, waste products are largely avoided, since the biomass does not end up as waste but is processed further to give a foodstuff of high value. As a result, the methods become less expensive due to the utilization of synergies.

In the present application, “highly pure” means a purity of the at least one carotenoid of at least 95%, preferably >95%, preferentially >96%, particularly preferably >97%, very particularly preferably >98%, most preferably >99%.

Suitable carotenoids which can be produced by the method of the invention are all natural and artificial carotenes and xanthophylls. The at least one carotenoid is in particular selected from the group consisting of astaxanthin, zeaxanthin, echinenone, β-cryptoxanthin, andonixanthin, adonirubin, canthaxanthin, 3-hydroxyechinenone, 3′-hydroxyechinenone, lycopene, β-carotene, lutein, phytofluene, bixin and phytoene. Preference is given here to astaxanthin or zeaxanthin. The carotenoids may be obtained by the method of the invention-individually or as mixtures of two or more of the abovementioned carotenoids. The carotenoid or carotenoids may be produced specifically, in particular when using the genetically modified organisms (GMO) indicated hereinbelow.

Foodstuffs are regarded as compositions used for nutrition. These also include compositions for supplementing nutrition. Animal feedstuffs and animal feed supplements, in particular, are regarded as foodstuffs.

After cultivation, the biomass can be removed from the culture broth according to variant a) of the preparation. To this end, any methods of solid/liquid separation familiar to and usually employable by the skilled worker may be used. These include in particular the mechanical processes, such as filtration and centrifugation, which are based on utilizing gravity, centrifugal force, pressure or vacuum. The processes and apparatus which may be used include in addition, inter alia, cross flow filtration or membrane techniques such as osmosis, reverse osmosis, microfiltration, ultrafiltration, nanofiltration, cake filtration processes (e.g. by means of automatic pressure filters (membrane, frame or chamber) filter presses, (agitated) pressure filters, suction filters, (vacuum) belt filters, (vacuum) drum filters, rotary filters, candle filters), centrifugation processes by means of continuously or batchwise operated centrifuges or filter centrifuges (e.g. inverting filter centrifuges, scraper centrifuges, pusher-type centrifuges, worm/screen centrifuges, slide centrifuges, separators or decanter centrifuges), processes utilizing gravity, such as flotation, sedimentation, sink-float purification and clarifying. The biomass is removed from the culture broth preferably by centrifugation by means of a decanter or by filtration by means of a membrane filtration unit.

The second step of the preparation according to variant b) generates a homogeneously distributed suspension of the solids in the culture broth. To this end, any methods familiar to and usually employable by the skilled worker may be used. Use is made here (on the laboratory scale) in particular of dispersers such as an UltraTurrax®. Cell disruption may be carried out but is not necessary.

The culture broth may, if necessary, be dehydrated in order to achieve a suitable solid content of between >2% and <50%. To this end, any methods of solid/liquid separation familiar to and usually employable by the skilled worker may be used. These include in particular the mechanical processes, such as filtration and centrifugation, which are based on utilizing gravity, centrifugal force, pressure or vacuum. The processes and apparatus which may be used include in addition, inter alia, cross flow filtration or membrane techniques such as osmosis, reverse osmosis, microfiltration, ultrafiltration, nanofiltration, cake filtration processes (e.g. by means of automatic pressure filters (membrane, frame or chamber) filter presses, (agitated) pressure filters, suction filters, (vacuum) belt filters, (vacuum) drum filters, rotary filters, candle filters), centrifugation processes by means of continuously or batchwise operated centrifuges or filter centrifuges (e.g. inverting filter centrifuges, scraper centrifuges, pusher-type centrifuges, worm/screen centrifuges, slide centrifuges, separators or decanter centrifuges), processes utilizing gravity, such as flotation, sedimentation, sink-float purification and clarifying. The biomass is removed from the culture broth preferably by centrifugation by means of a decanter or by filtration by means of a membrane filtration unit. The culture broth is subsequently dried. Again, it is possible to employ herein any processes and apparatus known to the skilled worker. Particularly suitable are apparatus for thermal drying such as convection, contact and radiation drying, for example tray, chamber, channel, flat web, plate, rotary drum, free-fall shaft, sieve belt, stream, fluidized bed, paddle, spherical bed, hotplate, thin film, can, belt, sieve drum, screw, tumble, contact disc, infrared, microwave and freeze driers, spray driers or spray driers with integrated fluidized bed, which are, if appropriate, heated by means of steam, oil, gas or electric current and, if appropriate, operated under reduced pressure. Depending on the apparatus, the mode of operation may be continuous or batchwise. Additionally or in combination therewith, the mechanical processes of solid/liquid separation already indicated above may be used.

However, granulation by extrusion, as disclosed by WO 97/36996 A2, is not necessary. The drying process renders the foodstuff stable and storable.

The culture broth is in particular spray-dried. Preference is given to using for the drying process spray drying as disclosed in DE 101 04 494 A1, DE-A-12 11 911 or EP 0 410 236 A1. In addition, reference is made to cf. Römpp Lexikon Chemie CD-ROM Version 2.0, Georg Thieme Verlag, 1999, “Sprühtrocknung” und Römpp Lexikon Biotechnologie, Georg Thieme Verlag, 1992, “Zerstäubungstrocknung” Spray drying has the advantage of a short dwell time of the product in the hot zone of the drier, thus achieving a particularly gentle drying process.

Intake temperatures of approx. 115° C.-180° C., preferably 120° C.-130° C., and exhaust temperatures of approx. 50° C.-80° C., preferably 55° C.-70° C., are chosen for spray drying. The preferred gas employed in the drying process is nitrogen.

It is possible, if appropriate, to add flow aids such as silicic acids etc., to achieve better flowability. The use of inert carrier materials, i.e. low molecular-weight inorganic carriers such as NaCl, CaCO₃, Na2SO4 or MgSO4, organic carriers such as glucose, fructose, sucrose, dextrins or starch products (rye, barley, oat flour, wheat semolina bran) is conceivable.

The dried product has a residual moisture of preferably less than 10%, preferably less than 5%, based on the dry weight. Its carotenoid content is between 0.05 and 20%, in particular 1 and 10%, based on the dry weight.

The foodstuff produced in this way may either be used directly or be processed by means of further additives, as is likewise disclosed in DE 101 04 494 A1.

According to the alternative IIC, the biomass, after it has been cultured and before it is being dried, is first removed from the culture broth. To this end, any solid/liquid separation methods familiar to and usually employable by the skilled worker, as already mentioned above for dehydration, may be employed. The biomass is removed from the culture broth preferably by centrifugation by means of a decanter or by membrane filtration.

Subsequently, the biomass is optionally washed with a solvent in which carotenoids are not soluble, in particular water, whereby in particular water-soluble components are removed. This step may, if appropriate, be supplemented using further solvents in which carotenoids are not soluble (e.g. alcohols), although this is not necessary within the scope of the invention and is not preferred, in order to avoid waste.

Subsequently, sterilization and subsequent or concomitant disruption of the cells in the biomass are carried out. Sterilization kills the microorganisms and stops enzyme activity which may be present. This is important for stability and for avoiding degradation of the biomass or the substances present therein, in particular the carotenoids.

Sterilization may be carried out using a customary method familiar to the skilled worker. This includes sterilization using steam, in particular at temperatures of more than 120° C. under pressure (≧1 bar) and for ≧approx. 20 min, and also treatment with high-energy radiation such as UV, microwave, gamma or beta rays. The sterilization within the framework of the method of the invention is preferably carried out using steam or microwave radiation.

The subsequent or concomitant cell disruption releases the carotenoids present in the cells. Cell disruption may likewise be carried out using any customary processes known to the skilled worker. These include mechanical and nonmechanical methods. The mechanical methods include dry milling, wet milling, stirring, homogenizing (e.g. in a high pressure homogenizer) and the use of ultrasound or microwaves. Suitable nonmechanical methods are physical, chemical and biochemical methods. These include short time heating, short time freezing, osmotic shock, drying, treatment with acids or bases and enzymic disruption. Advantageously, however, the process used for sterilization is used for cell disruption. Thus, preference is likewise given to carrying out cell disruption using steam or microwave radiation.

Sterilization and/or cell disruption may be carried out continuously or batchwise.

Sterilization and/or cell disruption may be carried out in the bioreactor used for cultivation or in other apparatus such as autoclaves etc. If the procedure is continuous, it is possible to use the microwave-using process disclosed in WO 01/83437 A1 and corresponding apparatus.

Prior to extraction, the biomass is, if appropriate, dried and/or homogenized. Again, it is possible to employ herein any customary processes and devices known to the skilled worker. Particularly suitable are apparatus for thermal drying such as convection, contact and radiation drying, for example tray, chamber, channel, flat web, plate, rotary drum, free-fall shaft, sieve belt, stream, fluidized bed, paddle, spherical bed, hotplate, thin film, can, belt, sieve drum, screw, tumble, contact disc, infrared, microwave and freeze driers, spray driers or spray driers with integrated fluidized bed, which are, if appropriate, heated by means of steam, oil, gas or electric current and, if appropriate, operated under reduced pressure. Depending on the apparatus, the mode of operation may be continuous or batchwise. Additionally or in combination therewith, the mechanical processes of solid/liquid separation already indicated above may be used.

However, granulation by extrusion, as disclosed by WO 97/36996 A2, is not necessary.

Subsequently, the carotenoids are partially extracted from the disrupted biomass by means of a carotenoid-dissolving solvent and separation of said solvent from the biomass. Both the solvent and the biomass then comprise carotenoids, the majority of said carotenoids being preferably present in the solvent.

The highly pure carotenoids are then isolated from the solvent, whereas the biomass is further processed to give a high quality, carotenoid-containing foodstuff which, due to the preceding cell disruption, also has good carotenoid bioavailability.

Accordingly, partial extraction means the deliberately incomplete extraction of the carotenoids from the biomass (cf. above). Preference is thus given to less than 100% of the total amount of carotenoids in the biomass being extracted from the latter by said extraction within the scope of the invention.

This is of great advantage, since complexity of the extraction increases disproportionately with the decreasing amount of carotenoid in the biomass.

The solvents used for extraction are ones which dissolve carotenoids such as, for example, hexane, ethyl acetate, dichloromethane or supercritical carbon dioxide. The preferred solvent used according to the invention is dichloromethane or supercritical carbon dioxide, it being possible, when using supercritical carbon dioxide, to subsequently transfer the carotenoids present therein to dichloromethane or to obtain the product of interest directly by expanding the carbon dioxide. In this connection, the amounts of solvents and the mixing times are chosen so that the desired amount of carotenoids is extracted from the biomass. More specifically, the extraction step is carried out only once, this being technically and economically sensible (cf. above).

The extraction may be carried out using any customary processes and apparatus. More specifically, liquid/liquid extraction is carried out if the biomass has been disrupted but not dried (carotenoid is present in liquid cell components in soluble form and is extracted therefrom), and solid/liquid extraction is carried out if the biomass has been dried. It is possible to use cold and hot extraction within particular temperature ranges, both continuous (e.g. Soxhlet extraction, perforation and percolation) and discontinuous processes which include, for example, extracting by shaking, with bases, by boiling, and digestion. They may also be carried out in a counterflow process.

It is possible to use for liquid/liquid extraction, for example, bubble columns, pulsating columns, columns with rotating internal fittings, mixer-settler batteries or stirred tanks etc.

Solid/liquid extraction may be carried out by means of customary apparatus. Preference is given to using stirred tanks or mixer-settler apparatus.

Alternatively, the cells may be disrupted without prior removal of the fermentation medium, followed by direct separation of a resultant carotenoid suspension from the biomass, for example by means of a decanter. The carotenoid suspension is subsequently taken up in dichloromethane and processed further or, alternatively, purified by washing with various aqueous solutions.

The highly pure carotenoids are isolated from the solvent by crystallizing said carotenoids from the solvent used and isolating the carotenoid crystals, in particular by filtration. The remaining mother liquor may, after distillation, reenter into the process, thus minimizing product losses despite low effort.

Crystallization may be carried out as usual. In addition, reference is made to cf. Römpp Lexikon Chemie CD-ROM Version 2.0, Georg Thieme Verlag, 1999, “Kristallisation”.

Crystallization is preferably carried out by gradually replacing the solvent with a solvent in which carotenoids are not soluble. Thus, carotenoid solubility is continuously decreased until said carotenoids precipitate in the form of pure crystals. Preference is given here to using a “lower alcohol” or water. Lower alcohol means aliphatic alcohols having from 1 to 4 carbon atoms. These include methanol, ethanol, propanol, isopropanol, 1-butanol, tert-butanol and sec-butanol. Preference is given to using methanol.

In this connection, the carotenoid solution may be heated, the temperature being kept preferably <100° C., in particular <60° C., so that dichloromethane is distilled off. It is also conceivable to use reduced pressure. The carotenoid crystals are then isolated and this may be carried out by the usual measures, in particular by filtration. If desired, further optional drying and/or purification steps may follow. These are, however, not necessary, since the carotenoid crystals are already highly pure.

The carotenoids are obtained as highly pure crystals and have a purity of at least 95%, preferably >95%, preferentially >96%, particularly preferably >97%, very particularly preferably >98%, most preferably >99%.

The achievable yields are between 45% and 95%, preferably between 70% and 95%, based on the amount present in the culture broth (0.5-15 g/L, preferably 1-10 g/L).

In order to further process the likewise carotenoid-containing biomass to give a high quality foodstuff, first residual solvents are removed from the carotenoid-containing biomass. This is preferably carried out by way of steam distillations or “stripping” with steam (cf. Römpp Lexikon Chemie CD-ROM Version 2.0, Georg Thieme Verlag, 1999, “Strippen”).

This may be followed, if appropriate, by homogeneously suspending the biomass in the culture broth removed above, in which case a solid content of >100 g/L and <600 g/L should be observed in order for the subsequent drying of the biomass or suspension for producing the foodstuff to be carried out without technical difficulties, i.e. the suspension must be pumpable. Suitable drying processes are all of the processes and apparatus already mentioned. More specifically, spray drying is used for the drying process which may be carried out as disclosed in DE 101 04 494 A1.

Intake temperatures of approx. 100° C.-180° C., preferably 120° C.-130° C., and exhaust temperatures of approx. 50° C.-80° C., preferably 55° C.-70° C., are chosen for spray drying. The preferred gas employed in the drying process is nitrogen.

The foodstuff produced in this way may either be used directly or be processed by means of further additives, as is likewise disclosed in DE 101 04 494 A1.

Foodstuffs are regarded as compositions used for nutrition. These also include compositions for supplementing nutrition. Animal feedstuffs and animal feed supplements, in particular, are regarded as foodstuffs. In addition, reference is made to Römpp Lexikon Chemie CD-ROM Version 2.0, Georg Thieme Verlag, 1999, “Nahrungsmittel”.

The dry product has a residual moisture of preferably less than 5%, based on dry weight. Its carotenoid content is between 0.05 and 20%, in particular 1 and 10%, based on dry weight. The desired carotenoid content can be controlled via the degree of extraction (cf. above).

Thus, foodstuffs obtainable by the method according to the invention include already after production large amounts of carotenoids which need not be added. Moreover, the nutrient content of said foodstuff is increased, due to the fact that it also contains biomass, in addition to the at least one carotenoid. The increase in nutrient content is particularly large according to the preferred alternative, since it includes, aside from the at least one carotenoid and biomass, in addition all media components of the fermentation. As a result, there are virtually no waste products, apart from aqueous media, which, however, can be purified without problems in a purification plant. In addition, the entire amount of carotenoids produced without or with only marginal losses is used, since no separation or work-up steps with heavy losses need to be carried out, in order to extract the total amount of carotenoid.

All of the solvents used in the above-described method of the invention are purified as far as possible and subsequently reused or reentered into the process. More specifically, the dichloromethane used is purified already during solvent replacement and is thereafter ready to be used again. The lower alcohol or methanol is purified, for example, by distillation and is likewise reused. The only waste produced is the distillation bottom which, together with the aqueous media, may be directed without risk to a purification plant where the actual waste produced in the end is only a small amount of sludge. Thus, the method

described is essentially waste-free. The invention is illustrated in more detail below on the basis of examples.

A) Cultivation of Blakeslea trispora

The following media were used for fermentation of Blakeslea trispora to produce the carotenoids:

Medium 1:

| | | |
|---|---|---|
| Glucose | 10.00 | g/l |
| Cotton seed oil | 30.00 | g/l |
| Soybean oil | 30.00 | g/l |
| Dextrin | 60.00 | g/l |
| Cottonseed meal | 75.00 | g/l |
| Triton X 100 | 1.20 | g/l |
| Ascorbic acid | 6.00 | g/l |
| Lactic acid | 2.00 | g/l |
| KH₂PO₄ | 0.50 | g/l |
| MnSO₄ × H2O | 100 | mg/l |
| Thiamine-HCl | 2 | mg/l |
| Isoniazide (isonicotinic acid hydrazide) | 0.75 | g/l |

The pH was adjusted to 6.5.

Medium 2:

| | | |
|---|---|---|
| Glucose | 20 | g/l |
| Asparagine | 2.00 | g/l |
| KH₂PO₄ | 5.00 | g/l |
| MgSO₄ × 7H₂O | 0.50 | g/l |
| CaCl₂ | 28 | mg/l |
| Thiamine-HCl | 1.00 | mg/l |
| Citric acid | 2.00 | mg/l |
| Fe(NO₃)₃ × 9H₂O | 1.50 | mg/l |
| ZnSO₄ × 7H₂O | 1.00 | mg/l |
| MnSO₄ × H₂O | 0.30 | mg/l |
| CuSO₄ × 5H₂O | 0.05 | mg/l |
| Na₂MoO₄ × 2H₂O | 0.05 | mg/l |

Medium 3

| | | |
|---|---|---|
| Glucose | 70.00 | g/l |
| Asparagine | 2.00 | g/l |
| Yeast extract | 1.00 | g/l |
| KH₂PO₄ | 1.50 | g/l |
| MgSO₄ × 7H₂O | 0.50 | g/l |
| Span 20 | 1.00 | g/l |
| Thiamine-HCl | 5.0 | mg/l |

The pH was adjusted to 5.5.

200 ml of the media described were inoculated in each case with spore suspensions of Blakeslea trispora ATCC 14272 Mating Type (−), which comprised 10⁸ (for Medium 2) and, respectively, 10⁷ (for Medium 1 and 3) spores. The cultivation was carried out in each case in 1 l Erlenmeyer flasks with baffles. With each medium, six identical flasks were prepared and incubated on a shaker at 28° C. and 140 rpm for 7 days.

B) Genetic Modification of Blakeslea Trispora

Materials and Methods

Molecular genetics work was carried out, unless described otherwise, by the methods in Current Protocols in Molecular Biology (Ausubel et al., 1999, John Wiley & Sons).

Strains and Growth Conditions

The Blakeslea trispora strains ATCC 14271 (mating type (+)) and ATCC14272 (−) (a wild type) were obtained, mating type (−)) were obtained from the American Type Culture Collection. B. trispora were grown in MEP medium (malt extract-peptone medium): 30 g/l malt extract (Difco), 3 g/l peptone (Soytone, Difco), 20 g/l agar, pH set to 5.5, ad 1000 ml with H₂O at 28° C.

Agrobacterium tumefaciens LBA4404 were grown according to Hoekema et al. (1983, Nature 303:179-180) at 28° C. for 24 h in agrobacterial minimal medium (AMM): 10 mM K₂HPO₄, 10 mM KH₂PO₄, 10 mM glucose, MM salts (2.5 mM NaCl, 2 mM MgSO₄, 700 μM CaCl₂, 9 μM FeSO₄, 4 mM (NH₄)₂SO₄).

Transformation of Agrobacterium tumefaciens

The plasmid pBinAHyg was electroporated into the agrobacterial strain LBA 4404 (Hoekema et al., 1983, Nature 303:179-180) (Mozo and Hooykaas, 1991, Plant Mol. Biol. 16:917-918). The following antibiotics were used for selection during agrobacterial growth: Rifampicin 50 mg/l (selection for the A. tumefaciens chromosome), streptomycin 30 mg/l (selection for the helper plasmid) and kanamycin 100 mg/l (selection for the binary vector).

Transformation of Blakeslea trispora

After 24 h of growth in AMM, the agrobacteria were diluted for transformation to an OD₆₀₀ of 0.15 in induction medium (IM: MM salts, 40 mM MES (pH 5.6), 5 mM glucose, 2 mM phosphate, 0.5% glycerol, 200 μM acetosyringone) and grown again in IM to an OD₆₀₀ of approx. 0.6 overnight.

For coincubation of Blakeslea ATCC 14271 or ATCC14272 and Agrobacterium, 100 μl of agrobacterial suspension were mixed with 100 μl of Blakeslea spore suspension (10⁷ spores/ml in 0.9% NaCl) and distributed in a sterile manner on a nylon membrane (Hybond N, Amersham) on IM-agarose plates (IM+18 g/l agar). After 3 days of incubation at 26° C., the membrane was transferred to an MEP-agar plate (30 g/l malt extract, 3 g/l peptone, pH 5.5, 18 g/l agar). To select for transformed Blakeslea cells, the medium comprised hygromycin at a concentration of 100 mg/l and, to select against agrobacteria, 100 mg/l cefotaxime. The incubation was carried out at 26° C. for approx. 7 days. This was followed by transferring mycelium to fresh selection plates. Resultant spores were rinsed with 0.9% NaCl and plated on CM17-1 agar (3 g/l glucose, 200 mg/l L-asparagine, 50 mg/l MgSO₄×7H₂O, 150 mg/l KH₂PO₄, 25 μg/l thiamine-HCl, 100 mg/l Yeast Extract, 100 mg/l sodium deoxycholate, 100 mg/L hygromycin, 100 mg/L cefotaxime, pH 5.5, 18 g/l agar). Individual genetically modified spores were isolated by putting them individually on selection medium, using an FACS instrument from BectonDickson (Modell Vantage+Diva Option).

Mutagenesis with MNNG

To reduce the number of nuclei per spore, spore suspensions were treated with MNNG (N-methyl-N′-nitro-N-nitrosoguanidine). For this, first a spore suspension containing 1×10⁷ spores/ml in Tris/HCl buffer, pH 7.0 was prepared. The spore suspension was admixed with MNNG at a final concentration of 100 μg/ml. The time of incubation in MNNG was chosen in such a way that the survival rate of the spores was approx. 5%. After incubation with MNNG, the spores were washed three times with 1 g/l Span 20 in 50 mM phosphate buffer pH 7.0 and plated.

Selection of Homonuclear Cells

Homonuclear Blakeslea trispora carB cells were selected in a manner similar to the experimental protocol for Phycomyces blakesleeanus (Roncero et al., 1984, Mutation Research, 125:195-204), modified by growth in the presence of 5-carbon-5-deazariboflavin (1 μg/ml) and Hygromycin 100 (μg/ml).

Preparation of Genetically Modified Blakeslea trispora by agrobacterium-Mediated Transformation

Preparation of the Recombinant Plasmid pBinAHyg

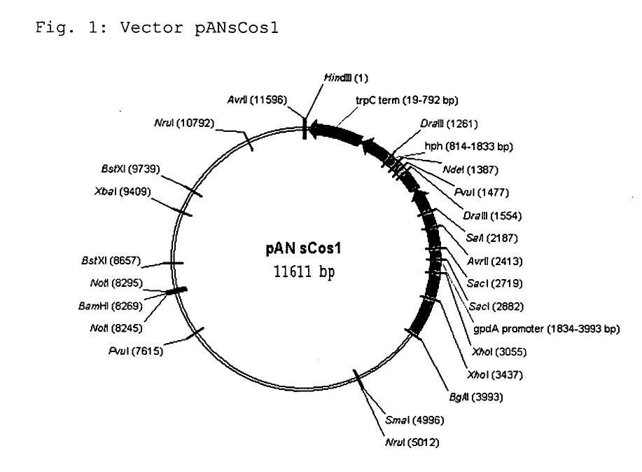

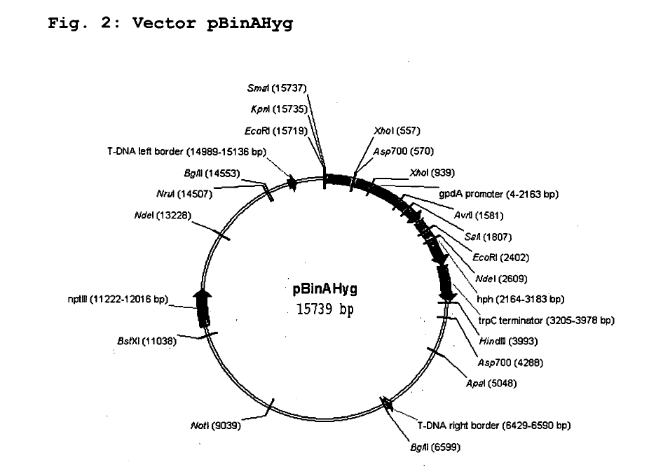

The gpdA-hph-trpC-cassette was isolated as BglII/HindIII fragment from the plasmid pANsCos1 (FIG. 1, Osiewacz, 1994, Curr. Genet. 26:87-90, SEQ ID NO: 4) and ligated into the binary plasmid pBin19 (Bevan, 1984, Nucleic Acids Res. 12:8711-8721) opened with BamHI/HindIII. The vector obtained in this way was referred to as pBinAHyg (FIG. 2, SEQ ID NO: 3) and comprised the E. coli hygromycin resistance gene (hph) under the control of the gpd promoter (SEQ ID NO: 1) and the trpC terminator (SEQ ID NO: 2) from Aspergillus nidulans and the corresponding border sequences required for Agrobacterium DNA transfer. The vectors mentioned in the exemplary embodiments described hereinbelow are pBinAHyg derivatives.

Transfer of pBinAHyg and pBinAHyg Derivatives into Agrobacterium tumefaciens

The transfer of the pBinAHyg plasmid into agrobacteria is described by way of example below. The derivatives were transferred in a similar manner.

The plasmid pBinAHyg was electroporated into the agrobacterial strain LBA 4404 (Hoekema et al., 1983, Nature 303:179-180) (Mozo and Hooykaas, 1991, Plant Mol. Biol. 16:917-918). The following antibiotics were used for selection during agrobacterial growth: Rifampicin 50 mg/l (selection for the A. tumefaciens chromosome), streptomycin 30 mg/l (selection for the helper plasmid) and kanamycin 100 mg/l (selection for the binary vector).

Transfer of pBinAHyg and pBinAHyg Derivatives into Blakeslea trispora

After 24 h of growth in AMM, the agrobacteria were diluted for transformation to an OD₆₆₀ of 0.15 in induction medium (IM: MM salts, 40 mM MES (pH 5.6), 5 mM glucose, 2 mM phosphate, 0.5% glycerol, 200 μM acetosyringone) and grown again in IM to an OD₆₆₀ of approx. 0.6 overnight.

For coincubation of Blakeslea trispora (B.t.) and Agrobacterium tumefasciens (A.t.) 100 μl of agrobacterial suspension were mixed with 100 μl of Blakeslea spore suspension (10⁷ spores/ml in 0.9% NaCl) and distributed in a sterile manner on a nylon membrane (Hybond N, Amersham) on IM-agarose plates (IM+18 g/l agar). After 3 days of incubation at 26° C., the membrane was transferred to an MEP-agar plate (30 g/l malt extract, 3 g/l peptone, pH 5.5, 18 g/l agar).

To select for transformed Blakeslea cells, the medium contained hygromycin at a concentration of 100 mg/l and, to select against agrobacteria, 100 mg/l cefotaxime. The incubation was carried out at 26° C. for approx. 7 days. This was followed by transferring mycelium to fresh selection plates. Resultant spores were rinsed with 0.9% NaCl and plated on CM17-1 agar (3 g/l glucose, 200 mg/l L-asparagine, 50 mg/l MgSO₄×7H₂O, 150 mg/l KH₂PO₄, 25 μg/l thiamine-HCl, 100 mg/l Yeast Extract, 100 mg/l sodium deoxycholate, pH 5.5, 100 mg/L cefotaxime, 100 mg/L hygromycine, 18 g/l agar). The transfer of spores to fresh selection plates was repeated three times. In this way, the transformant Blakeslea trispora GMO 3005 was isolated. Alternatively, the GMO (genetically modified organisms) were selected by applying the spores individually to CM-17 agar containing 100 mg/l cefotaxime, 100 mg/l hygromycin, by means of the BectonDickinson FacsVantage+Diva Option. In this case, fungal mycelium formed only where the spores had been genetically modified.

Detection of the Genetic Modification Due to Transfer of pBinAHyg and pBinAHyg Derivatives in Blakeslea trispora

Detection of the transfer is described by way of example below for pBinAHyg in Blakeslea trispora. Detection of the transfer of the derivatives was carried out in a similar manner.

200 ml of MEP medium (30 g/l malt extract, 3 g/l peptone, pH 5.5) were inoculated with 10⁵ to 10⁷ spores of the Blakeslea trispora GMO 3005 transformant and incubated on a rotary shaker at 200 rpm and 26° C. for 7 days. To detect successful transformation, DNA was isolated from the mycelium (Peqlab Fungal DNA Mini Kit) and used in a PCR (program: 94° C. for 1 min, then 30 cycles of 1 min. at 94° C., 1 min. at 58° C., 1 min. at 72° C., each).

The primers hph-forward (5′-CGATGTAGGAGGGCGTGGATA, SEQ ID NO: 5) and hph-reverse (5′-GCTTCTGCGGGCGATTTGTGT, SEQ ID NO: 6) were used for detecting the hygromycin resistance gene (hph). The expected hph fragment was 800 bp in length.

The primers nptIII-forward (5′-TGAGAATATCACCGGAATTG, SEQ ID NO: 7) and nptIII-reverse (5′-AGCTCGACATACTGTTCTTCC, SEQ ID NO: 8) were used for amplification of the kanamycin resistance gene nptIII and thus as a control for agrobacteria. The expected nptIII fragment was 700 bp in length.

The primers MAT292 (5′-GTGAATGGAAATCCCATCGCTGTC, SEQ ID NO: 9) and MAT293 (5′-AGTGGGTACTCTAAAGGCCATACC, SEQ ID NO: 10) were used for amplification of a fragment of the glycerinaldehyde 3-phosphate dehydrogenase gene gpd1 and thus as a control for Blakeslea trispora. The expected gpd1 fragment was 500 bp in length.

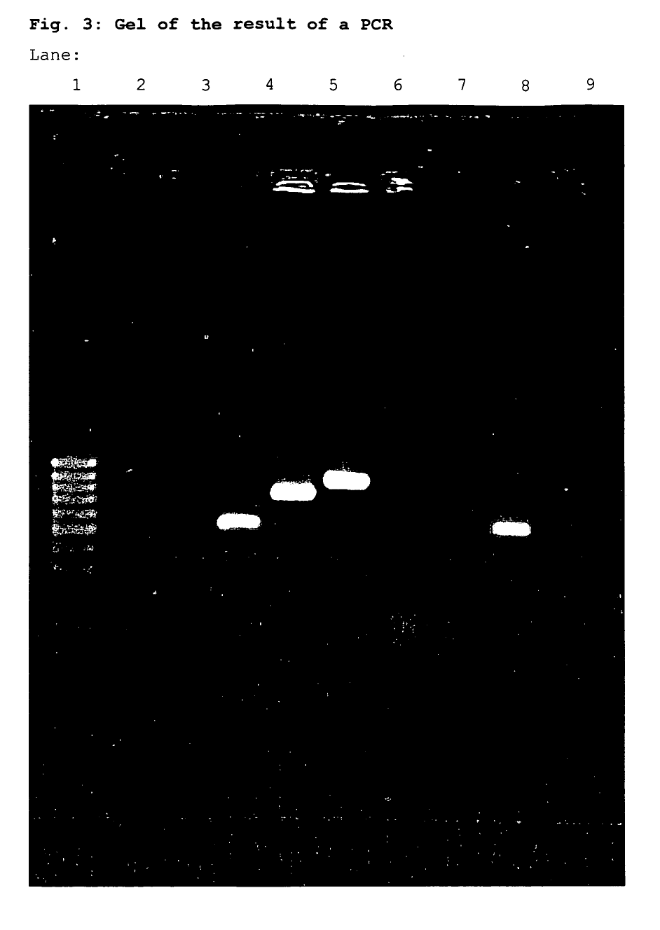

FIG. 3 depicts the result of the PCR of Blakeslea trispora DNA on the basis of a standard gel. The gel lanes were loaded as follows:

| 1) | 100 bp size marker(100 bp-1 kb) | |
|---|---|---|
| 2) | B.t. GMO 3005 | primer nptIII-for/nptIII-rev |
| 3) | B.t. GMO 3005 | primer hph-for/hph-rev |
| 4) | B.t. GMO 3005 | primer MAT292/MAT293 (gpd) |
| 5) | A.t. with pBinAHyg plasmid | primer nptIII-for/nptIII-rev |
| 6) | A.t. with pBinAHyg plasmid | primer hph-for/hph-rev |
| 7) | B.t. 14272 WT | primer nptIII-for/nptIII-rev |
| 8) | B.t. 14272 WT | primer hph-for/hph-rev |
| 9) | B.t. 14272 WT | primer MAT292/MAT293 (gpd) |

The hygromycin resistance gene (hph) and, as a positive control, the glycerinaldehyde 3-phosphate dehydrogenase gene (gpd1) were detected in Blakeslea trispora DNA. In contrast, nptIII was not detected.

Thus, the genetic modification of Blakeslea trispora by Agrobacterium-mediated transformation was detected.

Isolation of Homokaryotic Blakeslea trispora GMOs:Preparation of Homonuclear Strains

The successful transfer of the pBinAHyg vector and pBinAHyg derivatives into Blakeslea trispora produces genetically modified organisms. In GMO of Blakeslea trispora. However, Blakeslea has multinuclear cells at all stages of the vegetative and sexual cell cycle. Therefore, vector foreign DNA is usually inserted only in one nucleus. It is, however, the aim to obtain Blakeslea strains in which vector foreign DNA has been inserted in all nuclei, i.e. the aim is a homonuclear recombinant fungal mycelium.

In order to prepare homokaryotic cells of this kind, spore suspensions of the recombinant strains were first treated with MNNG. For this, first a spore suspension containing 1×10⁷ spores/ml in Tris/HCl buffer, pH 7.0 was prepared. The spore suspension was admixed with MNNG at a final concentration of 100 μg/ml. The time of incubation in MNNG was chosen in such a way that the survival rate of the spores was approx. 5%. After incubation with MNNG, the spores were washed three times with 1 g/l Span 20 in 50 mM phosphate buffer pH 7.0 and plated.

1) Preparation of Homonuclear Recombinant Strains by Means of FACS (Fluorescence-Activated Cell Sorting)

A small proportion of the spores of Blakeslea trispora or of the genetically modified Blakeslea trispora strains is by nature mononuclear. To produce homonuclear recombinant strains comprising the foreign DNA of pBinAHyg or pBinAHyg derivatives, the mononuclear spores were sorted out by means of FACS and plated on MEP (30 g/l malt extract, 3 g/l peptone, pH 5.5, 18 g/l agar) containing 100 mg/l cefotaxime and 100 mg/l hygromycin. The mycelia produced here were homonuclear. For FACS, the spores of a 3 day old smear were washed off with 10 ml of Tris-HCl 50 mMol+0.1% Span20 per agar plate. The spore concentration was from 0.5 to 0.8×10⁷ spores per ml. 1 ml of DMSO and 10 μl of Syto 11 (dye stock solution in DMSO, Molecular Probes No. S-7573) were added to 9 ml of spore suspension. This was followed by staining at 30° C. for 2 h. Selection and application were carried out by means of a BectonDickinson FacsVantage+Diva Option. First, a size selection was carried out in order to separate individual spores from aggregates and contaminations. These spores were then applied sorted according to their fluorescence (excitation=488 nm emission=530 nm). The left shoulder of the Gauss curve of the fluorescence frequency distribution contained the mononuclear spores.

The spores were then plated on MEP agar plates and new spores were generated.

These spores were plated on medium comprising 5-carbon-5-deazariboflavin and, additionally, hygromycin, in a similar manner to the protocol by Roncero et al.

This enabled homokaryotic cells of the genotype hyg^{R} and dar⁻ to be selected.

2) Preparation of Homonuclear Strains by Reducing the Number of Nuclei and Selection with FACS

To reduce the number of nuclei per spore, spore suspensions were treated with MNNG (N-methyl-N′-nitro-N-nitrosoguanidine) prior to selection, thus achieving a reduction in the number of nuclei by chemical mutagenesis.

For this, first a spore suspension containing 1×10⁷ spores/ml in Tris/HCl buffer, pH 7.0 was prepared. The spore suspension was admixed with MNNG at a final concentration of 100 μg/ml. The time of incubation in MNNG was chosen in such a way that the survival rate of the spores was approx. 5%. After incubation with MNNG, the spores were washed three times with 1 g/l Span 20 in 50 mM phosphate buffer pH 7.0 and sorted and selected by the method described under 1).

As an alternative, it was also possible to reduce the number of nuclei in the spores by using X-rays and UV rays, as described by Cerdá-Olmedo and Patricia Reau in Mutation Res., 9(1970), 369-384.

3) Preparation of Homonuclear Strains by Selection for Recessive Selection Markers

A suitable recessive selection marker for selection of homonuclear mycelia is, for example, the recessive selection marker pyrG. Wild-type strains of Blakeslea trispora are pyrG⁺. These strains are unable to grow in the presence of the pyrimidine analog 5-fluoroorotate (FOA), because they convert FOA to lethal metabolites via orotidine 5′-monophosphate decarboxylase. Genetically modified pyrG⁻ homonuclear Blakeslea lack the enzyme activity of orotidine 5′-monophosphate decarboxylase. Consequently, these pyrG⁻ strains are unable to utilize 5-fluoroorotate. Therefore, these strains grow in the presence of FOA and uracil. If the pyrG⁻ mutation and the foreign DNA insert are coupled on the nucleus of a mononuclear spore, this spore may form homonuclear recombinant fungal mycelium.

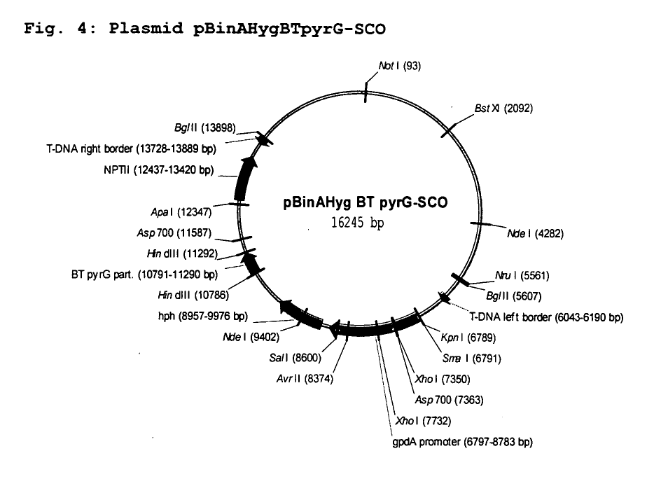

First, the plasmid pBinAHygBTpyrG-SCO (SEQ ID NO: 36, FIG. 4) was generated by inserting a fragment of pyrG (SEQ ID NO: 65) from Blakeslea trispora into pBinAHyg. Said plasmid was transformed into Blakeslea trispora and caused pyrG disruption there due to homologous recombination.

Homonuclear Blakeslea trispora GMO with the pyrG phenotype were selected as follows. Plating on MEP (30 g/l malt extract, 3 g/l peptone, pH 5.5, 18 g/l agar) containing 100 mg/l cefotaxime and 100 mg/l hygromycin for agrobacterium-mediated transformation of pBinAHygBTpyrG-SCO was carried out as described above. The spores of the transformants were washed off with 10 ml of Tris-HCl 50 mM+0.1% Span20 per agar plate. The spore concentration was from 0.5 to 0.8×10⁷ spores per ml. The spores were then plated on FOA medium containing 100 mg/l cefotaxime and 100 mg/l hygromycin. FOA medium comprised, per liter, 20 g of glucose, 1 g of FOA, 50 mg of uracil, 200 ml of citrate buffer (0.5 M, pH 4.5) and 40 ml of trace salt solution according to Sutter, 1975, PNAS, 72:127). Homonuclear pyrG⁻ mutants exhibited growth on the uracil-containing FOA medium but no growth when plated on FOA medium without uracil. In the same way, homonuclear GMO were prepared from the Blakeslea trispora GMO described below for producing xanthophylls.

Alternatively, it is possible to plate the spores according to the protocol by Roncero et al. on medium comprising 5-carbon-5-deazariboflavin and, additionally, hygromycin (Roncero et al., 1984, Mutation Research, 125: 195-204). This enables homokaryotic cells of the genotype hyg^{R} and dar⁻ to be selected.

According to this principle, homokaryotic Blakeslea trispora strains with the phenotype hyg^{R} and dar⁻ are generated.

Exemplary Embodiments for Preparing Genetically Modified Organisms of Blakeslea trispora for Producing Carotenoids and Carotenoid Precursors.

The plasmids mentioned below were generated by the “overlap-extension PCR” method and by subsequent insertion of the amplification products into the pBinAHyg plasmid. The overlap-extension PCR method was carried out as described in Innis et al. (Eds) PCR protocols: a guide to methods and applications, Academic Press, San Diego. Transformation of the pBinAHyg derivatives and preparation of homonuclear genetically modified Blakeslea trispora strains were carried out as described above.

Genetically Modified Blakeslea trispora Strains for Producing Zeaxanthin

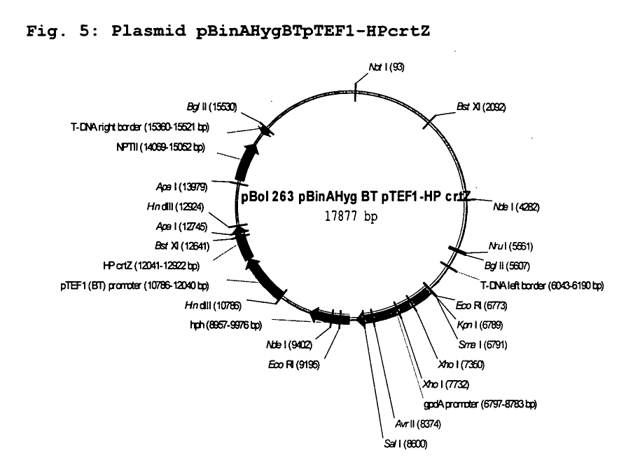

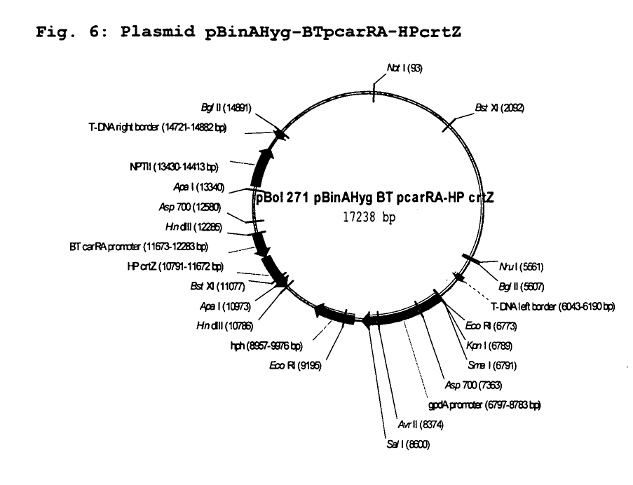

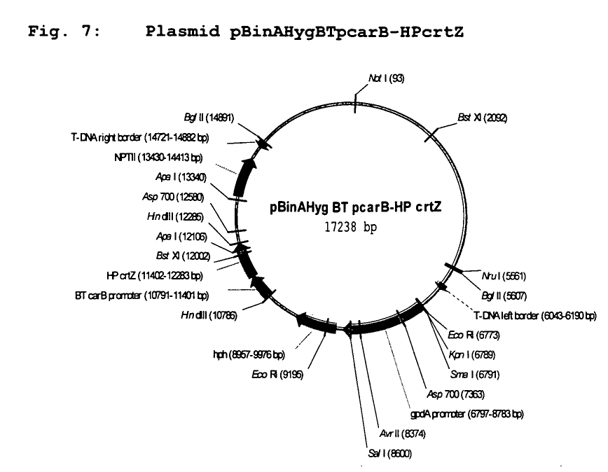

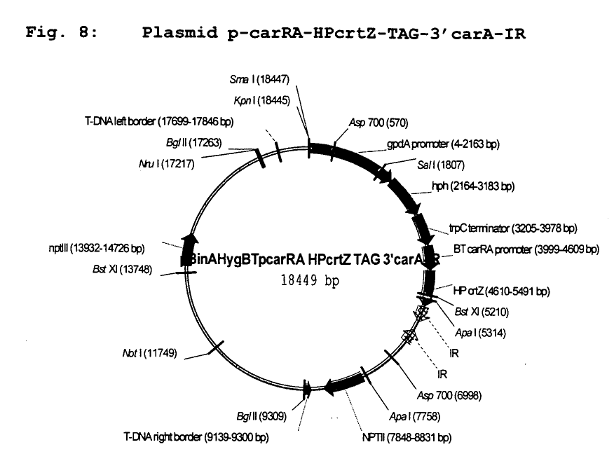

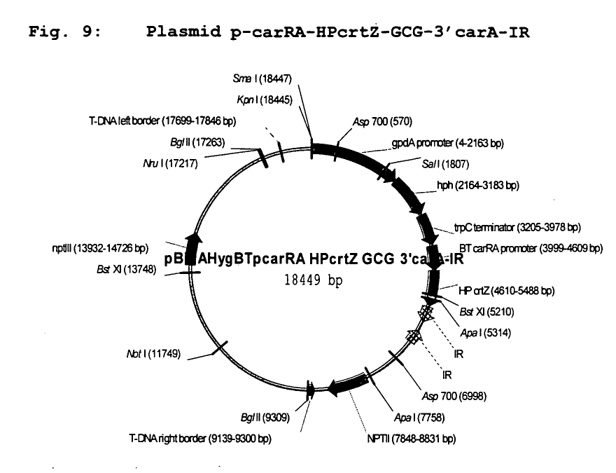

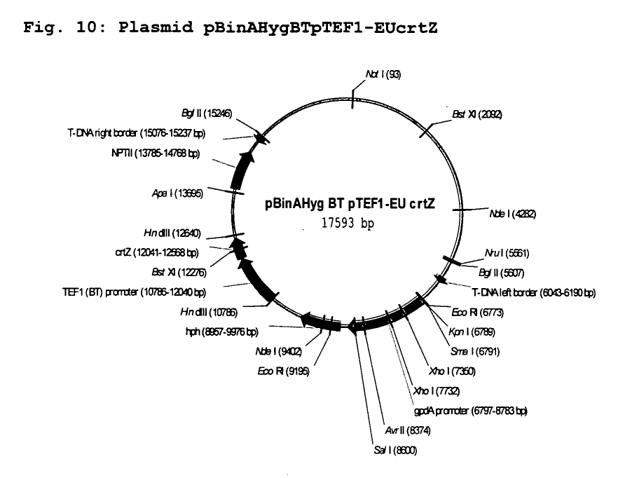

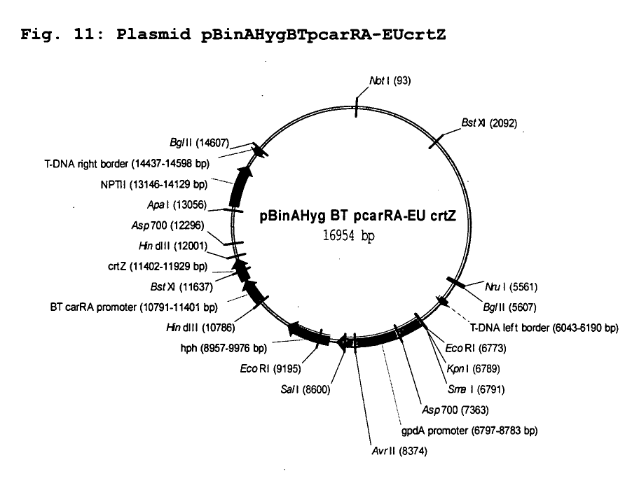

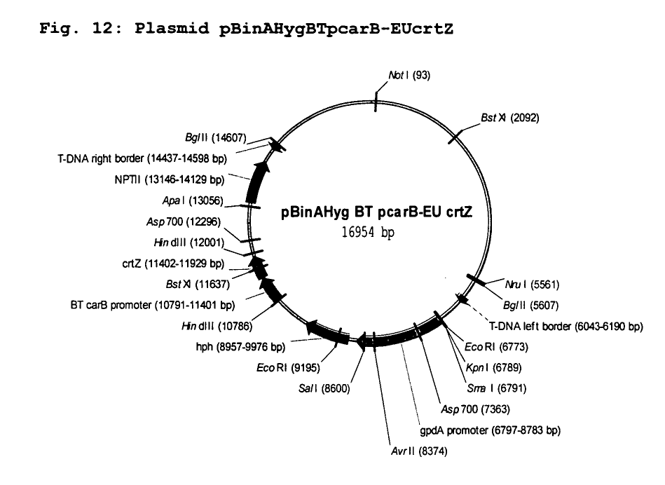

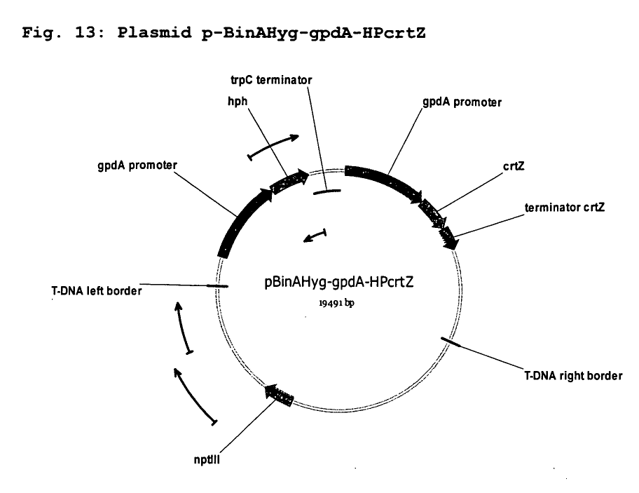

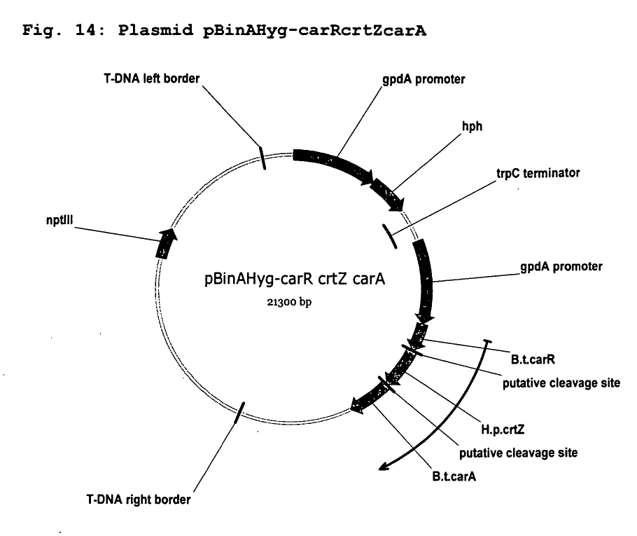

The following plasmids (pBinAHyg derivatives) were used for genetic modification of Blakeslea trispora for the production of zeaxanthin, and thus encode inter alia hydroxylases (crtZ):
ptef1-HPcrtZ, comprising the gene of the HPcrtZ hydroxylase (SEQ ID NO:70) from Haematococcus pluvialis Flotow NIES-144 (Accession No. AF162276) under the control of the Blakeslea trispora ptef1 promoter (Seq. pBinAHygBTpTEF1-HPcrtZ, SEQ ID NO:37, FIG. 5);
p-carRA-HPcrtZ, comprising the gene of the HPcrtZ hydroxylase from Haematococcus pluvialis Flotow NIES-144 under the control of the Blakeslea trispora pcarRA promoter (Seq. pBinAHygBTpcarRA-HPcrtZ, SEQ ID NO:38, FIG. 6);
p-carB-HPcrtZ, comprising the gene of the HPcrtZ hydroxylase from Haematococcus pluvialis Flotow NIES-144 under the control of the Blakeslea trispora pcarB promoter (Seq. pBinAHygBTpcarB-HPcrtZ, SEQ ID NO:39, FIG. 7);
p-carRA-HPcrtZ-TAG-3′carA-IR, comprising the gene of the HPcrtZ hydroxylase from Haematococcus pluvialis Flotow NIES-144 under the control of the Blakeslea trispora pcarRA promoter. An inverted repeat structure is located downstream of the hydroxylase gene, which structure is derived from the 3′ end of carA and the region downstream of carA (IR, SEQ ID NO:74, “Inverted Repeat 1” approx. 350 bp of carA, then approx. 200 bp “Loop” and then approx. 350 bp “Inverted Repeat 2”) (Seq. pBinAHyg-BTpcarRA-HPcrtZ-TAG-3′carA-IR, SEQ ID NO:40, FIG. 8);
p-carRA-HPcrtZ-GCG-3′carA-IR, comprising the gene of the HPcrtZ hydroxylase from Haematococcus pluvialis Flotow NIES-144 under the control of the Blakeslea trispora pcarRA promoter. The hydroxylase gene is fused to an inverted repeat structure which is derived from the 3′ end of carRA and the region downstream of carA (IR, SEQ ID NO:74, “Inverted Repeat 1” approx. 350 bp of carA, then approx. 200 bp “Loop” and then approx. 350 bp “Inverted Repeat 2”). Consequently, the derived fusion protein consists of the Haematococcus pluvialis hydroxylase and the carboxy terminus of Blakeslea trispora CarA (Seq. pBinAHyg-BTpcarRA-HPcrtZ-GCG-3′carA-IR, SEQ ID NO:41, FIG. 9).
p-tef1-EUcrtZ, comprising the gene of the EUcrtZ hydroxylase (SEQ ID NO:71) from Erwinia uredova 20D3 (Accession No. D90087) under the control of the ptef1 promoter (Seq. pBinAHygBTpTEF1-EUcrtZ, SEQ ID NO:42, FIG. 10);
p-carRA-EUcrtZ, comprising the gene of the EUcrtZ hydroxylase from Erwinia uredova 20D3 under the control of the Blakeslea trispora pcarRA promoter (Seq. pBinAHygBTpcarRA-EUcrtZ, SEQ ID NO:43, FIG. 11);
p-carB-EUcrtZ, comprising the gene of the EUcrtZ hydroxylase from Erwinia uredova 20D3 under the control of the Blakeslea trispora pcarB promoter (Seq. pBinAHygBTpcarB-EUcrtZ, SEQ ID NO:44, FIG. 12);
p-gpdA-HPcrtZ-t-crtZ, comprising the gene of the HPcrtZ hydroxylase from Haematococcus pluvialis Flotow NIES-144 under the control of the gpdA promoter and the t-crtZ terminator; i.e. of the sequence section downstream of crtZ from Haematococcus pluvialis Flotow NIES-144 (SEQ ID NO:73) (Seq. pBinAHyg-gpdA-HPcrtZ-tcrtZ, SEQ ID NO:43, FIG. 13).
p-gpdA-BTcarR-HPcrtZ-BTcarA, comprising a gene fusion of genes of lycopine cyclase carR from Blakeslea trispora, of HPcrtZ hydroxylase from Haematococcus pluvialis Flotow NIES-144 and of the phytoene synthase carA from Blakeslea trispora and under the control of the Aspergillus nidulans gpdA promoter (Seq. pBinAHyg-carR_crtZ_carA, SEQ ID NO:46, FIG. 14).
   Preparation of Genetically Modified Blakeslea trispora Strains for Producing Canthaxanthin

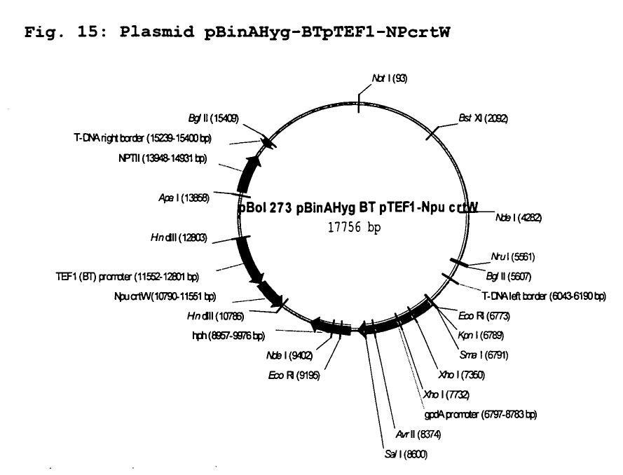

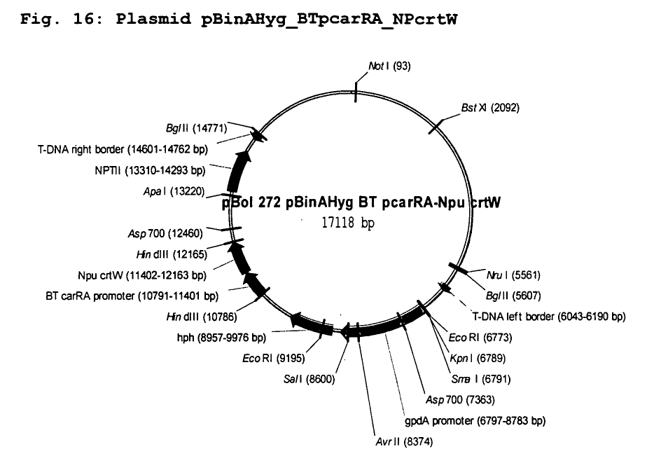

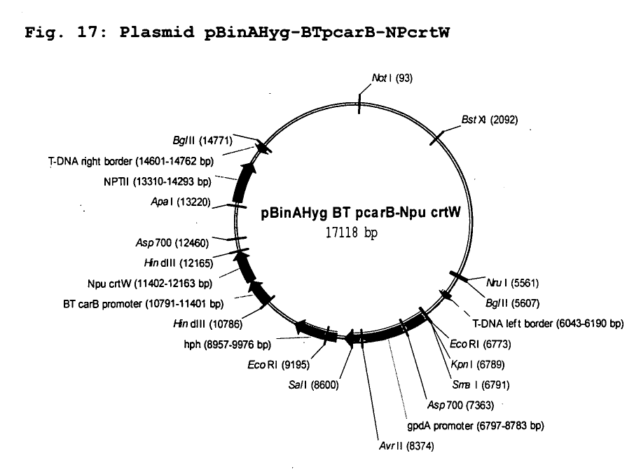

The following plasmids (pBinAHyg derivatives) were used for genetic modification of Blakeslea trispora for the production of canthaxanthin, and thus encode inter alia ketolases (crtW):
p-tef1-NPcrtW, comprising the gene of the NPcrtW ketolase (SEQ ID NO:72) from Nostoc punctiforme PCC73102 (ORF148, Accession No. NZ_AABC01000196) and under the control of the Blakeslea trispora ptef1 promoter (Seq. pBinAHygBTpTEF1-NpucrtW, SEQ ID NO:47, FIG. 15);
p-carRA-NPcrtW, comprising the gene of the NPcrtW ketolase from Nostoc punctiforme PCC73102 and under the control of the Blakeslea trispora pcarRA promoter (Seq. pBinAHygBTpcarRA-NpucrtW, SEQ ID NO:48, FIG. 16);
p-carB-NPcrtW, comprising the gene of the NPcrtW ketolase from Nostoc punctiforme PCC73102 and under the control of the Blakeslea trispora pcarB promoter (Seq. pBinAHygBTpcarB-NpucrtW, SEQ ID NO:49, FIG. 17).
   Preparation of Genetically Modified Blakeslea trispora Strains for Producing Astaxanthin

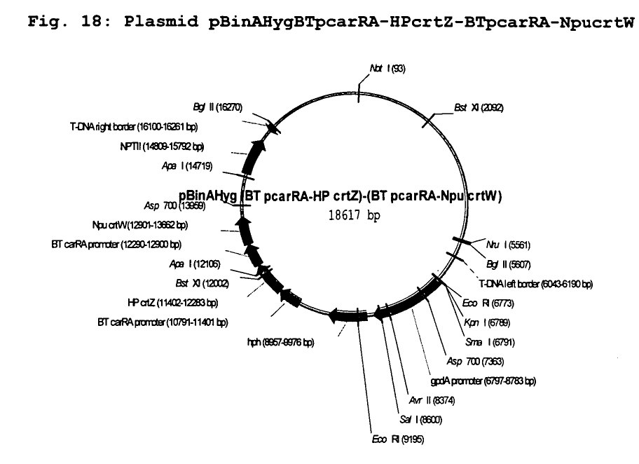

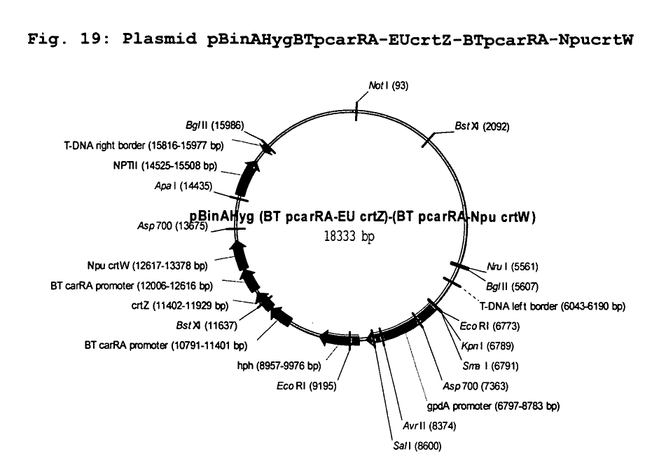

The following plasmids (pBinAHyg derivatives) were used for genetic modification of Blakeslea trispora for producing astaxanthin, i.e. encode inter alia hydroxylases (crtZ) and ketolases (crtW):
p-carRA-HPcrtZ-pcarRA-NPcrtW, comprising the gene of the HPcrtZ hydroxylase from Haematococcus pluvialis Flotow NIES-144 and the gene of the NPcrtW ketolase from Nostoc punctiforme PCC73102 (ORF148, Accession No. NZ-AABC01000196), both in each case under the control of the Blakeslea trispora pcarRA promoter (Seq. pBinAHygBTpcarRA-HPcrtZ-BTpcarRA-NpucrtW, SEQ ID NO:50, FIG. 18);
p-carRA-EUcrtZ-pcarRA-NPcrtW, comprising the gene of the EUcrtZ hydroxylase from Erwinia uredova 20D3 (Accession No. D90087) and the gene of the NPcrTW ketolase from Nostoc punctiforme PCC73102, both in each case under the control of the Blakeslea trispora pcarRA promoter (Seq. pBinAHygBTpcarRA-EUcrtZ-BTpcarRA-NpucrtW, SEQ ID NO:51, FIG. 19).
   Cloning and Sequence Analysis of Genes and Promoters which May be Utilized by Way of Example for Genetic Modification of Blakeslea trispora.

Cloning and sequencing of various Blakeslea trispora genes and promoters are described by way of example below.

Cloning and Sequence Analysis of ptef1

Blakeslea trispora p-tef was cloned on the basis of a sequence, previously published in GenBank, of the structural gene of Blakeslea trispora translation elongation factor 1-α (AF157235). Starting from the sequence entry AF157235 primers were selected for inverted PCR in order to amplify and sequence the promoter region upstream of said structural gene. In the inverted nested PCR of 200 ng of XhoI-cleaved and circularized genomic DNA of Blakeslea trispora ATCC14272, a 3000-bp fragment was obtained in the following reaction mixture: template DNA (1 μg of genomic DNA of Blakeslea trispora ATCC 14272) primers MAT344 5′-GGCGTACTTGAAGGAACCCTTACCG-3′ (SEQ ID NO: 63) and MAT 345 5′-ATTGATGCTCCCGGTCACCGTGATT-3′ (SEQ ID NO: 64), 0.25 μM each, 100 μM dNTP, 10 μl of Herculase polymerase buffer 10×, 5 U of Herculase (addition at 85° C.), H₂O ad 100 μl. The PCR profile was as follows: 95° C., 10 min (1 cycle); 85° C., 5 min (1 cycle); 60° C., 30 s, 72° C., 60 s, 95° C., 30 s (30 cycles); 72° C., 10 min (1 cycle). The sequence section upstream of the putative start codon of the tef1 gene in the 3000-bp fragment was referred to as ptef1 promoter.

Cloning, Sequence Analysis of the Gene of HMG-CoA Reductase from Blakeslea trispora

First, the cosmid vector pANsCos1 was used for preparing a gene library of Blakeslea trispora ATCC 14272, Mating Type (−). The vector was linearized by cleavage with XbaI and then dephosphorylated. Further cleavage with BamHI generated the insertion site into which the Blakeslea trispora genomic DNA, partially cleaved with Sau3AI and dephosphorylated, was ligated. The cosmids produced in this way were subsequently packaged in vitro and transferred into Escherichia coli.

On the basis of the known sequence of a fragment of the Blakeslea trispora gene encoding HMG-CoA reductase (Eur. J. Biochem 220, 403-408 (1994)), a 315-bp DNA probe was prepared by the following PCR. Reaction mixture: 1 μg of genomic DNA of Blakeslea trispora ATCC 14272, primers MAT314 5′-CCGATGGCGACGACGGAAGGTTGTT-3′ [SEQ ID NO: 79] and MAT315 5′-CATGTTCATGCCCATTGCATCACCT-3′ [SEQ ID NO: 80], 0.25 μM each, 100 μM dNTP, 10 μl of Herculase polymerase buffer 10×, 5 U of Herculase (addition at 85° C.), H₂O ad 100 μl. The PCR profile was as follows: 95° C., 10 min (1 cycle); 85° C., 5 min (1 cycle); 58° C., 30 s, 72° C., 30 s, 95° C., 30 s (30 cycles); 72° C., 10 min (1 cycle).

This DNA probe was used for screeing the cosmid gene library. A clone whose cosmid hybridized with said DNA probe was identified. The insert of this cosmid was sequenced. The DNA sequence comprised a section which was assigned to the gene of an MHG-CoA reductase [HMG-CoA-Red.gb].

Cloning and Sequence Analysis of carB

(carB=Blakeslea trispora phytoene desaturase gene) The degenerated primers MAT182 5′-GCNGARGGNATHTGGTA-3′ (SEQ ID 52) and MAT192 5′-TCNGCNAGRAADATRTTRTG-3′ (SEQ ID 53) were derived from comparing the peptide sequences of phytoene desaturases and comparing the corresponding DNA sequences of Phycomyces blakesleeanus, Cercospora nicotianae, Phaffia rhodozyma and Neurospora crassa. The PCR was carried out in 100 μl reaction mixtures. These comprised 200 ng of genomic DNA of Blakeslea trispora ATCC14272, 1 μM MAT182, 1 μM MAT192, 100 μM dNTP, 10 μl of Pfu polymerase buffer 10×, 2.5 U of Pfu polymerase (addition at 85° C.), H₂O ad 100 μl.

The PCR profile was as follows: 95° C., 10 min (1 cycle); 85° C., 5 min (1 cycle); 40° C., 30 s, 72° C., 30 s, 95° C., 30 s (35 cycles); 72° C., 10 min (1 cycle).

This resulted in a 358-bp fragment whose derived peptide sequence is similar to the phytoene desaturase sequences. The method of inverted PCR (Innis et al. in PCR protocols: a guide to methods and applications. 1990. pp. 219-227) was used for amplifying, cloning and sequencing, according to the principle of chromosome walking, the gene regions upstream and downstream of the 350-bp fragment as follows:
(i) a 1.1 kbp fragment, by PCR with the primers MAT219 5′-AAGTGACACCGGTTACACGCTTGTCTT-3′ (SEQ ID 54) and MAT 220 5′-GCTTATCACCATCTGTTACCTCCTTGC-3′ (SEQ ID 55), obtained from 200 ng of EcoRI-cleaved and circularized genomic DNA of Blakeslea trispora ATCC14272, 0.25 μM MAT219, 0.25 μM MAT220, 100 μM dNTP, 10 μl of Herculase polymerase buffer 10×, 5 U of Herculase (addition at 85° C.), H₂O ad 100 μl. The PCR profile as as follows: 95° C., 10 min (1 cycle); 85° C., 5 min (1 cycle); 60° C., 30 s, 72° C., 60 s, 95° C., 30 s (30 cycles); 72° C., 10 min (1 cycle),
(ii) a 2.9 kbp fragment, by PCR with the primers MAT219 and MAT220, obtained from 200 ng of XbaI cleaved and circularized genomic DNA Blakeslea trispora ATCC14272, 0.25 μM MAT219, 0.25 μM MAT220, 100 μM dNTP, 10 μl of Herculase polymerase buffer 10×, 5 U of Herculase (addition at 85° C.), H₂O ad 100 μl. The PCR profile was as follows: 95° C., 10 min (1 cycle); 85° C., 5 min (1 cycle); 60° C., 30 s, 72° C., 3 min, 95° C., 30 s (30 cycles); 72° C., 10 min (1 cycle).

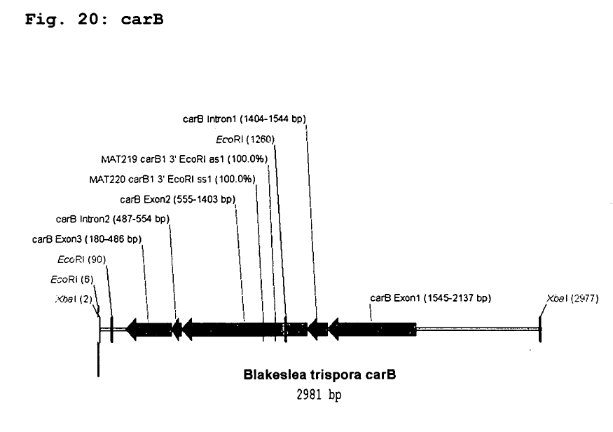

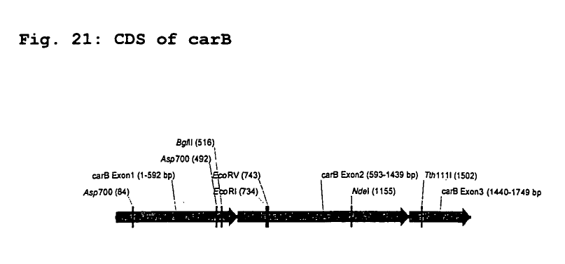

FIG. 20 depicts diagrammatically the cloned sequence section. Sequencing was carried out in strand and counterstrand orientation, using the cloned fragments and the PCR products. FIG. 21 depicts the sequence of the cloned sequence section.

Sequence Comparisons

The nucleotide sequence of carB and the peptide sequence of the derived protein CarB were compared with the known sequences of related proteins. The sequences were compared using the GAP and BESTFIT programs.

CarB—Identical Aminoacyl Residues According to GAP

Program Settings:

| | |
|---|---|
| Gap weight: | 8 |
| Length weight: | 2 |
| Average match: | 2.912 |
| Average mismatch: | −2.003 |

The following values, in %, of amino acid correspondence to CarB of Blakeslea trispora ATCC14272 were found:

| | |
|---|---|
| Phycomyces blakesleeanus: | 72.491 |
| Phaffia rhodozyma: | 50.460 |
| Neurospora crassa: | 47.943 |
| Cercospora nicotianae: | 47.740 |

CarB—Identical Aminoacyl Residues According to BESTFIT

Program Settings:

| | |
|---|---|
| Gap weight: | 8 |
| Length weight: | 2 |
| Average match: | 2.912 |
| Average mismatch: | −2.003 |

The following values, in %, of amino acid correspondence to CarB of Blakeslea trispora ATCC14272 were found:

| | |
|---|---|
| Phycomyces blakesleeanus: | 73.380 |
| Phaffia rhodozyma: | 53.175 |
| Neurospora crassa: | 51.896 |
| Cercospora nicotianae: | 50.791 |

carB—Identical Bases According to GAP

Program Settings:

| | |
|---|---|
| Gap weight: | 50 |
| Length weight: | 3 |
| Average match: | 10.000 |
| Average Mismatch: | 0.000 |

The following values, in %, of base correspondence to CarB of Blakeslea trispora ATCC14272 were found:

| | |
|---|---|
| Phycomyces blakesleeanus: | 64.853 |
| Cercospora nicotianae: | 50.143 |
| Phaffia rhodozyma: | 43.179 |
| Neurospora crassa: | 42.130 |

carB—Identical Bases According to BESTFIT

Program Settings:

| | |
|---|---|
| Gap weight: | 50 |
| Length weight: | 3 |
| Average match: | 10.000 |
| Average mismatch: | −9.000 |

The following values, in %, of base correspondence to CarB of Blakeslea trispora ATCC14272 were found:

| | |
|---|---|
| Phycomyces blakesleeanus: | 68.926 |
| Phaffia rhodozyma: | 62.403 |
| Neurospora crassa: | 60.230 |
| Cercospora nicotianae: | 56.884 |

Cloning for carB Expression

In order to clone and express Blakeslea trispora carB, the possible protein sequences were derived in six reading frames from the above-described cloned sequence section from Blakeslea trispora. These protein sequences were compared with the sequences of the phytoene desaturates from Phycomyces blakesleeanus, Phaffia rhodozyma, Neurospora crassa, Cercospora nicotianae. On the basis of the sequence comparison, three exons were identified in the cloned sequence section of the Blakeslea trispora genomic DNA, which, put together, result in a coding region whose derived gene product has, over its entire length, 72.7% identical aminoacyl residues with the CarB phytoene desaturase of Phycomyces blakesleeanus. This sequence section comprising three possible exons and two possible introns was therefore referred to as gene carB. In order to check the predicted gene structure, the coding sequence of Blakeslea trispora carB was generated by means of PCR using Blakeslea trispora cDNA as template and the primers Bol1425 5′-AGAGAGGGATCCTTAAATGCGAATATCGTTGC-3′ (SEQ ID 56) and Bol1426 5′-AGAGAGGGATCCATGTCTGATCAAAAGAAGCA-3′ (SEQ ID 57). The DNA fragment obtained was sequenced. The location of exons and introns was confirmed by comparing the cDNA with the genomic carB DNA. FIG. 21 depicts diagrammatically the coding sequence of carB. For expression of carB in Escherichia coli, first the NdeI cleavage site in carB was removed by the overlap extension PCR method and an NdeI cleavage site was introduced at the 5′ end of the gene and a BamHI cleavage site was introduced at the 3′ end. The DNA fragment obtained was ligated with the vector pJOE2702. The plasmid obtained was referred to as pBT4 and cloned together with pCAR-AE into Escherichia coli XL1-Blue. Expression was induced with rhamnose. The enzyme activity was detected by way of detecting lycopine synthesis via HPLC. The cloning steps are described below:

PCR 1.1:

Approx. 0.5 μg of Blakeslea trispora cDNA, 0.25 μM MAT350 5′-ACTTTATTGGATCCTTAAATGCGAATATCGTTGCTGC-3′ (SEQ ID 58), 0.25 μM MAT244 5′-GTTCCAATTGGCCACATGAAGAGTAAGACAGGAAACAG-3′ (SEQ ID 59), 100 μM dNTP, 10 μl of Pfu polymerase buffer (10×), 2.5 U of Pfu polymerase (addition at 85° C., “hot start”) and H₂O ad 100 μL.

Temperature Profile:

1. 95° C. 10 min, 2. 85° C. 5 min, 3. 40° C. 30 s, 4. 72° C. 1 min 30 s, 5. 95° C. 30 s, 6. 50° C. 30 s, 7. 72° C. 1 min 30 s, 8. 95° C. 30 s, 9. 72° C. 10 min

Cycles: (1-2.) 1×, (3-5.) 5×, (6-8.) 25×, (9.) 1×

PCR 1.2:

Approx. 0.5 μg of Blakeslea trispora cDNA, 0.25 μM MAT243 5′-CCTGTCTTACTCTTCATGTGGCCAATTGGAACCAACAC-3′ (SEQ ID 60), 0.25 μM MAT353 5′-CTATTTTAATCATATGTCTGATCAAAAGAAGCATATTG-3′ (SEQ ID 61), 100 μM dNTP, 10 μl of Pfu polymerase buffer (10×), 2.5 U of Pfu polymerase (addition at 85° C., “hot start”) and H₂O ad 100 μL.

Temperature Profile:

1. 95° C. 10 min, 2. 85° C. 5 min, 3. 40° C. 30 s, 4. 72° C. 1 min 30 s, 5. 95° C. 30 s, 6. 50° C. 30 s, 7. 72° C. 1 min 30 s, 8. 95° C. 30 s, 9. 72° C. 10 min

Cycles: (1-2.) 1×, (3-5.) 5×, (6-8.) 25×, (9.) 1×

Purification of the PCR Fragments from PCR 1.1, 1.2

For this purpose, PCR 2 was carried out to prepare the coding sequence of Blakeslea trispora carB for cloning into pJOE2702:

Approx. 50 ng of PCR 1.1 product and approx. 50 ng of PCR 1.2 product, with 0.25 μM MAT350 5′-ACTTTATTGGATCCTTAAATGCGAATATCGTTGCTGC-3′ (SEQ ID NO 58), 0.25 μM MAT353 5′-CTATTTTAATCATATGTCTGATCAAAAGAAGCATATTG-3′ (SEQ ID NO 61), 100 μM dNTP, 10 μL of Pfu polymerase buffer (10×), 2.5 U of Pfu polymerase (addition at 85° C., “hot start”) and H₂O ad 100 μL.

Temperature Profile:

1. 95° C. 10 min, 2. 85° C. 5 min, 3. 59° C. 30 s, 4. 72° C. 2 min, 5. 95° C. 30 s, 6. 72° C. 10 min

Cycles: (1-2.) 1×, (3-5.) 22×, (6.) 1×

Subsequently, the fragment obtained (˜1.7 kbp) was purified, followed by ligation into the vector pPCR-Script-Amp, cloning into Escherichia coli XL1-Blue, sequencing of the insert, cleavage with NdeI and BamHI and ligation into pJOE2702. The plasmid obtained was referred to as pBT4.

Characterization and Detection of the Enzyme Activity of CarB (Phytoene Desaturase)

The gene product derived from carB was referred to as CarB. CarB has the following properties, based on peptide sequence analysis:

| | |
|---|---|
| Length: | 582 aminoacyl residues |
| Molecular mass: | 66470 |
| Isoelectric point: | 6.7 |
| Catalytic activity: | Phytoene desaturase |
| Reactant: | Phytoene |
| Product: | Lycopene |
| EC number: | EC 1.14.99- |

The enzyme activity was detected in vivo. Transfer of the plasmid (pCAR-AE) into Escherichia coli XL1-Blue produces the strain Escherichia coli XL1-Blue (pCAR-AE). This strain synthesizes phytoene. An additional transfer of the pBT4 plasmid into Escherichia coli XL1-Blue produces the strain Escherichia coli XL1-Blue (pCAR-AE)(pBT4). Since an enzymicly active phytoene desaturase is formed starting from carB, this strain produces lycopene.

The plasmids pCAR-AE and pBT4 were therefore transferred into Escherichia coli. The carotenoids were extracted from the cells grown in liquid culture and characterized (cf. above).

HPLC analysis revealed that the Escherichia coli XL1-Blue (pCAR-AE) strain produces phytoene and the Escherichia coli XL1-Blue (pCAR-AE)(pBT4) strain produces lycopene. Consequently, CarB has the enzyme activity of a phytoene desaturase.

Preparation of Genetically Modified Blakeslea trispora Strains for Producing Phytoene

The preparation of genetically modified organisms for producing phytoene is described by way of example below.

Vector pBinAHygΔcarB for Generating carB⁻ Mutants of Blakeslea trispora

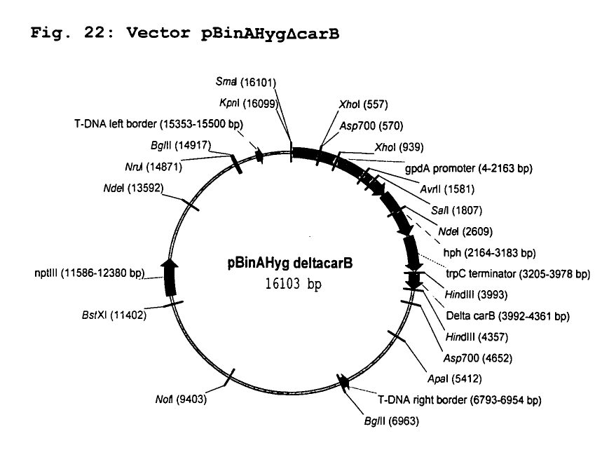

The vector pBinAHygΔcarB (SEQ. ID. NO:62, FIG. 22) was constructed to delete carB in Blakeslea trispora. The precursor of pBinAHygΔcarB is pBinAHyg (SEQ. ID. NO:3, FIG. 2) which was constructed as follows:

The gpdA-hph cassette was isolated as BglII/HindIII fragment from the plasmid pANsCos1 (SEQ. ID. NO:4, FIG. 1, Osiewacz, 1994, Curr. Genet. 26:87-90) and ligated into the BamHI/HindIII-opened binary plasmid pBin19 (Bevan, 1984, Nucleic Acids Res. 12:8711-8721). The vector obtained in this way was referred to as pBinAHyg and comprises the E. coli hygromycin resistance gene (hph) under the control of the gpd promoter and the trpc terminator from Aspergillus nidulans and the appropriate border sequences required for the Agrobacterium DNA transfer.

The carB coding sequence was amplified by means of PCR using the primers MAT350 (SEQ ID NO 58) and MAT353 (SEQ ID NO 61) and the following parameters: 50 ng of pBT4 with 0.25 μM MAT350 5′-ACTTTATTGGATCCTTAAATGCGAATATCGTTGCTGC-3′, 0.25 μM MAT353 5′-CTATTTTAATCATATGTCTGATCAAAAGAAGCATATTG-3′, 100 μM dNTP, 10 μl of Pfu polymerase buffer, 2.5 U of Pfu polymerase (addition at 85° C., “hot start”) and H₂O to 100 μl

Temperature Profile:

1. 95° C. 10 min, 2. 85° C. 5 min, 3. 58° C. 30 s, 4. 72° C. 2 min, 5. 95° C. 30 s, 6. 72° C. 10 min.

Cycles: (1.-2.) 1×, (3-5.) 30×, (6.) 1×

The fragment obtained (˜1.7 kbp) was subsequently purified, followed by cleavage with HindIII, further purification of the 364 bp HindIII fragment carB, followed by cleavage of pBinAHyg with HindIII, ligation of the 364 bp HindIII fragment carB into pBinAHyg, transformation of the vector into Escherichia coli and isolation of the construct and referred to as pBinAHygΔcarB, as described above. Alternatively, partial cleavage with HindIII was carried out and a larger carB HindIII fragment was cloned into pBinAHyg to produce pBinAHygΔcarB.

Generation of carB⁻ Mutants of Blakeslea trispora

The pBinAHygΔcarB plasmid was first transferred into the Agrobacterium strain LBA 4404, for example by electroporation (cf. above). The plasmid was subsequently transferred from Agrobacterium tumefaciens LBA 4404 in Blakeslea trispora ATCC 14272 and in Blakeslea trispora ATCC 14271 (cf. above). Successful detection of the gene transfer into Blakeslea trispora was carried out via polymerase chain reaction according to the following protocol:

approx. 0.5 μg of DNA from Blakeslea trispora ATCC 14272 carB⁻ or ATCC 14271 carB⁻ was reacted with 0.25 μM primer hph forward 5′-CGATGTAGGAGGGCGTGGATA-3′ (SEQ ID NO 5), 0.25 μM primer hph reverse 5′-GCTTCTGCGGGCGATTTGTGT-3′ (SEQ ID NO 6), 100 μM dNTP, 10 μL of Herculase polymerase buffer, 2.5 U of Herculase DNA polymerase (addition at 85° C., “hot start”) and H₂O to 100 μl.

Temperature Profile:

1. 95° C. 10 min, 2. 85° C. 5 min, 3. 58° C. 1 min, 4. 72° C. 1 min, 5. 94° C. 1 min, 6. 72° C. 10 min.

Cycles: (1.-2.) 1×, (3-5.) 30×, (6.) 1×.

It was attempted to amplify the Agrobacterium kanamycin resistance gene as a negative control. For this purpose, the following PCR conditions were used: approx. 0.5 μg of DNA from Blakeslea trispora ATCC 14272 carB⁻ and ATCC 14271 carB⁻ was reacted with 0.25 μM primer nptIII forward 5′-TGAGAATATCACCGGAATTG-3′ (SEQ ID NO 7), 0.25 μM primer nptIII reverse 5′-AGCTCGACATACTGTTCTTCC-3′ (SEQ ID NO 8), 100 μM dNTP, 10 μL of Herculase polymerase buffer, 2.5 U of Herculase DNA polymerase (addition at 85° C., “hot start”) and H₂O to 100 μl.

Temperature Profile:

1. 95° C. 10 min, 2. 85° C. 5 min, 3. 58° C. 1 min, 4. 72° C. 1 min, 5. 94° C. 1 min, 6. 72° C. 10 min—

Cycles: (1-2.) 1×, (3-5.) 30×, (6.) 1×

C) Production of Carotenoids and Carotenoid Precursors by Blakeslea trispora

The carotenoids zeaxanthin, canthaxanthin, astaxanthin and phytoene were produced by fermenting the corresponding genetically modified Blakeslea trispora (+) and (−) strains, detecting the carotenoid produced by means of HPLC analysis and isolating it.

The liquid medium for producing carotenoids comprised, per liter: 19 g of cornflour, 44 g of soybean flour, 0.55 g of KH₂PO₄, 0.002 g of thiamine hydrochloride, 10% sunflower oil. The pH was adjusted to 7.5 with KOH.

To produce the carotenoids, shaker flasks were inoculated with spore suspensions of (+) and (−) strains of the Blakeslea trispora GMO. The shaker flasks were incubated at 26° C. and 250 rpm for 7 days. Alternatively, trisporic acids were added to mixtures of the strains after 4 days, followed by 3 more days of incubation. The final concentration of the trisporic acids was 300-400 μg/ml.

Extraction and Analysis

Extraction:

1. Removal of 10 ml of culture suspension
2. Centrifugation, 10 min, 5000×g
3. Discarding of the supernatant
4. Resuspension of the pellet in 1 ml of tetrahydrofuran (THF) by vortexing
5. Centrifugation, 5 min, 5000×g
6. Removal of the THF phase
7. Repetition of steps 4.-6. (2×)
8. Pooling of the THF phases
9. Centrifugation of the pooled THF phases at 20 000×g for 5 min in order to remove residual aqueous phase.
   Analysis

Phytoene Measurement by Means of HPLC

| | |
|---|---|
| Column: | ZORBAX Eclipse XDB-C8, 5 um, 150*4.6 mm |
| Temperature: | 40° C. |
| Flow rate: | 0.5 ml/min |
| Injection volume: | 10 μl |
| Detection: | UV 220 nm |
| Stop time: | 12 min |
| Post run time: | 0 min |
| Maximum pressure: | 350 bar |
| Eluent A: | 50 mM NaH₂PO₄, pH 2.5 with perchloric acid |
| Eluent B: | Acetonitrile |

Gradient:

| Time [min] | A [%] | B [%] | Flow [ml/min] |
|---|---|---|---|
| 0 | 50 | 50 | 0.5 |
| 12 | 50 | 50 | 0.5 |

Extracts of the fermentation broth were used as matrix. Prior to HPLC, each sample was filtered through a 0.22 μm filter. The samples were kept cool and protected from light. In each case 50-1000 mg/l were weighed and dissolved in THF for calibration. The standard used was phytoene which has a retention time of 7.7 min under the given conditions.

Measurement of Lycopene, β-Carotene, Echinenone, Canthaxanthin, Cryptoxanthin, Zeaxanthin and Astaxanthin by Means of HPLC

| | |
|---|---|
| Column: | Nucleosil 100-7 C18, 250*4.0 mm (Macherey & |
| | Nagel) |
| Temperature: | 25° C. |
| Flow rate: | 1.3 ml/min |
| Injection volume: | 10 μl |
| Detection: | 450 nm |
| Stop time: | 15 min |
| Post run time: | 2 min |
| Maximum pressure: | 250 bar |
| Eluent A: | 10% acetone, 90% H₂O |
| Eluent B: | Acetone |

Gradient:

| Time [min] | A [%] | B [%] | Flow [ml/min] |
|---|---|---|---|
| 0 | 30 | 70 | 1.3 |
| 10 | 5 | 95 | 1.3 |
| 12 | 5 | 95 | 1.3 |
| 13 | 30 | 70 | 1.3 |

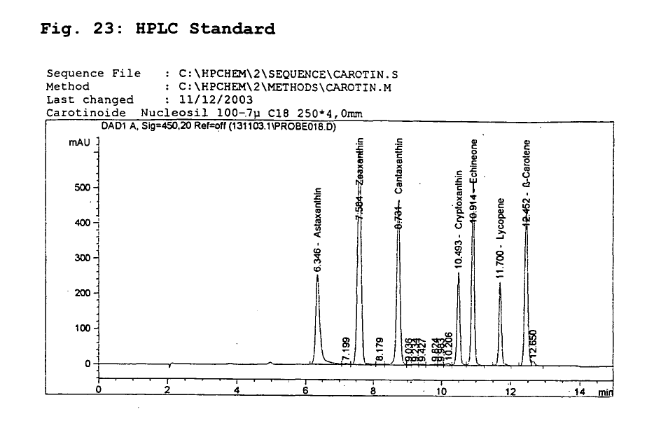

Extracts of the fermentation broth were used as matrix. Prior to HPLC, each sample was filtered through a 0.22 μm filter. The samples were kept cool and protected from light. In each case 10 mg were weighed and dissolved in 100 ml of THF for calibration. The following carotenoids with the following retention times were used as standard: β-carotene (12.5 min), lycopene (11.7 min), echinenone (10.9 min), cryptoxanthin (10.5 min), canthaxanthin (8.7 min), zeaxanthin (7.6 min) and astaxanthin (6.4 min) [see FIG. 23].

Production of Zeaxanthin by Genetically Modified Blakeslea trispora Strains

Production of zeaxanthin by genetically modified organisms (GMO) of Blakeslea trispora is described by way of example below.

The vector pBinAHygBTpTEF1-HPcrtZ was transferred into Blakeslea trispora by Agrobacterium-mediated transformation (see above). A hygromycin-resistant clone was isolated and transferred to a potato-glucose agar plate (Merck KGaA, Darmstadt, Germany).

Starting from this plate, a spore suspension was prepared after three days of incubation at 26° C. A 250 ml Erlenmeyer flask without baffles and comprising 50 ml of growth medium (47 g/l cornflour, 23 g/l soybean flour, 0.5 g/l KH₂PO₄, 2.0 mg/l thiamine-HCl, pH adjusted to 6.2-6.7 with NaOH before sterilization) was inoculated with 1×10⁵ spores. This preculture was incubated at 26° C. and 250 rpm for 48 hours. For the main culture, a 250 ml Erlenmeyer flask without baffles and comprising 40 ml of production medium was inoculated with 4 ml of the preculture and incubated at 26° C. and 150 rpm for 8 days. The production medium comprised 50 g/l glucose, 2 g/l caseine acid hydrolysate, 1 g/l yeast extract, 2 g/l L-asparagine, 1.5 g/l KH₂PO₄, 0.5 g/l MgSO₄×7H₂O, 5 mg/l thiamine-HCl, 10 g/l Span20, 1 g/l Tween 80, 20 g/l linoleic acid, 80 g/l corn steep liquor. After 72 hours, kerosene was added at a final concentration of 40 g/l.

After harvesting the cultures, the remaining culture volume of approximately 35 ml was increased to 40 ml with water. Subsequently, the cells were disrupted in a high pressure homogenizer, type Micron Lab 40, APV Gaulin, 3× at 1500 bar.

The suspension comprising the disrupted cells was admixed with 35 ml of THF and incubated with shaking at 250 rpm and RT in the dark for 60 min. Then 2 g of NaCl were added and the mixture was incubated with shaking once more. The extraction mixture was then centrifuged at 5000×g for 10 min. The colored THF phase was removed and the cell mass was completely colorless.

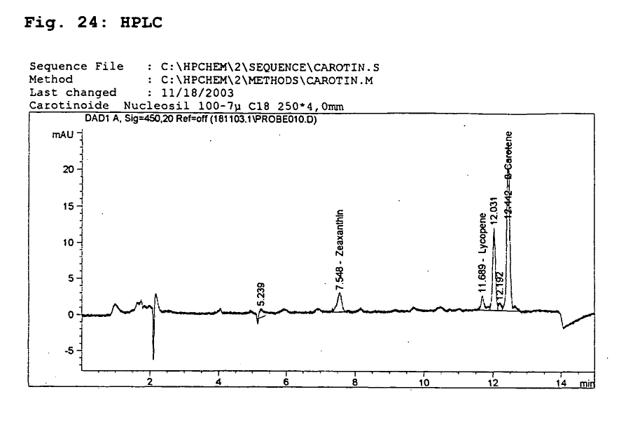

The THF phase was concentrated to 1 ml in a rotary evaporator at 30 mbar and 30° C. and then taken up again in 1 ml of THF. After centrifugation at 20 000×g for 5 min, an aliquot of the upper phase was removed and analyzed by HPLC (FIG. 24, FIG. 23).

D) Work-Up and Isolation of the Carotenoids and the Foodstuff

The culture broths indicated above under A) were worked up as follows in order to obtain highly pure carotenoids and a corresponding foodstuff.

The carotenoid content of the culture broths 1, 2, 3 was between 0.5 and 1.5 g/l.

D1) Example According to Variant a) IIA and Variant b) IIA or IIB

The cultures having identical media (approx. 1 l in total) were combined at the end of the cultivation period and homogenized with the aid of a disperser (Ultra.Turrax®).

The concentration of solids in the media 1 and 2 was 37 g/l and 11 g/l, respectively. The culture broth was dehydrated using a centrifuge. If the cell concentrations and the solids content of the medium are high, the culture broth may also be processed further without prior solid-liquid separation (medium 3: 127 g of solid/l). After previous homogenization using a disperser (Ultra-Turrax®) and with constant stirring of the suspension, the cell mass was applied via a peristaltic pump to the dryer. Injection into the cylinder of the laboratory spray dryer was carried out via a two-component nozzle having a diameter of 2.0 mm, with 2 bar and 4.5 Nm³/h of nitrogen. The intake temperature was approx. 125° C. to 127° C. The drying gas was nitrogen at a flow rate of 22 Nm³/h. The exhaust temperature was between 59° C. and 61° C. For each of the three fermentation broths it was possible to precipitate flowable product on the cyclone of the spray dryer. The wall films in the chamber (where present) flaked off the vessel wall automatically and are classified as unproblematic.

Between 8 and 100 g of powdery foodstuff were obtained which could be used directly as animal feedstuff. It comprised approx. 1-10% carotenoids based on dry weight. The residual moisture was less than 5%.

Example According to Variant b) IIC

D2) Extraction with Tetrahydrofuran

The cells of in each case 40 ml of culture broths 1, 2, 3 were disrupted 3× at 1500 bar by a high pressure homogenizer, type Micron Lab 40, APV Gaulin. In each case 20 ml of the suspensions comprising the disrupted cells were admixed with 20 ml of tetrahydrofuran and incubated with shaking at 30° C. in a rotary shaker at 200 rpm for 30 min. Then 2 g of NaCl were added and the phases were separated by centrifuging at 5000×g for 5 min. The THF phase was removed. Subsequently, the aqueous phase was extracted once more with 20 ml of THF. The extracts were combined. The carotenoid concentration was quantified by HPLC.

D3) Extraction with Dichloromethane

The biomass was removed from the culture broth (200 ml) by centrifugation at 5000×g in a laboratory centrifuge for 10 min.

The removed wet biomass (in each case approx. 10 g to 100 g) was admixed with 10-100 ml of water in order to remove water-soluble components. The biomass was removed (laboratory centrifuge) and then sterilized with steam (T=121, t=30 min, 1 bar) in an autoclave, whereby the cells were disrupted.

25-250 g of dichloromethane were added to the cell debris and the carotenoid was extracted from the biomass by shaking. The biomass was removed in a laboratory centrifuge.

A solvent exchange from dichloromethane to methanol was carried out, for which the carotenoid solution was kept at 40° C. to 60° C. for approx. 4 hours and, over this period, admixed continuously with a total volume of 20-200 ml of methanol. Dichloromethane was recovered as solvent in the process. First carotenoid crystals precipitated. Subsequently, the solution was slowly cooled to approx. 10° C. over 6 h, with the size and number of carotenoid crystals increasing. The mother liquor was then filtered off and the carotenoid crystals were dried. Part of the mother liquor may be reused for solvent exchange. The other part is distilled and the methanol purified in this way is reused in the solvent exchange.

0.0.08 g to 0.24 g of carotenoid crystals were obtained whose purity (HPLC, cf. above) was 95%. The yield of carotenoid crystals was 80% based on the concentration of carotenoid in the biomass.

The removed, dichloromethane-wet biomass, after steam distillation, was spray-dried (T_{I}=125° C., T_{E}=60° C.) and may be used as animal feed supplement.

To this end, the cell mass, after previous homogenization using a disperser (Ultra-Turrax) and with constant stirring of the suspension, was applied via a peristaltic pump to the dryer.

Injection into the cylinder of the laboratory spray dryer was carried out via a two-component nozzle having a diameter of 2.0 mm, with 2 bar and 4.5 Nm³/h nitrogen. The intake temperature was approx. 125° C. to 127° C. The drying gas was nitrogen at a flow rate of 22 Nm³/h. The exhaust temperature was between 59° C. and 61° C. For each of the three fermentation broths it was possible to precipitate flowable product on the cyclone of the spray dryer. The wall films in the chamber (where present) flaked off the vessel wall automatically and were classified as unproblematic.

Approx. 2.5-25 g of powdery foodstuff were obtained which could be used directly as animal feedstuff. It comprised approx. 0.5%-1.5% carotenoids based on dry weight. The residual moisture was less than 5%.

The total yield of carotenoid (including the purified carotenoid foodstuff) was approx. 95% based on the starting amount of carotenoid in the culture broth.

1. A method for producing carotenoids or their precursors using genetically modified organisms of the Blakeslea genus, which method comprises the following steps:
(i) transformation of at least one of the cells,
(ii) homokaryotic conversion of the cells obtained in step (i) to produce cells in which one or more genetic characteristics of the nuclei are all modified in an identical manner and said genetic modification manifests itself in the cells, and
(iii) selection and reproduction of the genetically modified cell or cells,
(iv) cultivation of the genetically modified cells,
(v) preparation of the carotenoid produced by the genetically modified cells or the carotenoid precursor produced by said genetically modified cells.

2. The method according to claim 1, wherein the cells are from fungi of the Blakeslea trispora species.

3. The method according to claim 1, wherein a vector or free nucleic acids are used in the transformation of step (i).

4. The method according to claim 3, wherein the vector employed in the transformation is integrated into the genome of at least one of the cells.

5. The method according to claim 4, wherein the vector employed in the transformation comprises a promoter and/or a terminator.

6. The method according to claim 3, wherein a vector comprising a gpd, pcarB, pcarRA and/or ptef1 promoter and/or a trpC terminator is employed in the transformation.

7. The method according to claim 3, wherein a vector comprising a resistance gene is employed in the transformation.

8. The method according to claim 7, wherein the vector employed in the transformation comprises a hygromycin resistance gene (hph.

9. The method according to claim 6, wherein the gpd promoter comprises the sequence SEQ ID NO: 1.

10. The method according to claim 6, wherein the trpC terminator comprises the sequence SEQ ID NO: 2.

11. The method according to claim 6, wherein the ptef1 promoter comprises the sequence SEQ ID NO: 35.

12. The method according to claim 6, wherein the gpd promoter and the trpC terminator are derived from Aspergillus nidulans.

13. The method according to claim 3, wherein the vector comprises the sequence SEQ ID NO: 3.

14. The method according to claim 1, wherein the transformation is carried out using agrobacteria, conjugation, chemicals, electroporation, bombardment with DNA-loaded particles, protoplasts or microinjection.

15. The method according to claim 1, wherein a mutagenic agent is employed in the homokaryotic conversion of step (ii).

16. The method according to claim 15, wherein the mutagenic agent employed is N-methyl-N′-nitronitrosoguanidine (MNNG), UV radiation or X rays.

17. The method according to claim 1, wherein the selection is carried out by labeling and/or selecting the mononuclear cells.

18. The method according to claim 1, wherein 5-carbon-5-deazariboflavin (darf) and hygromycin (hyg) or 5-fluororotate (FOA) and uracil and hygromycin are employed in the selection.

19. The method according to claim 3, wherein the vector employed in the transformation includes genetic information for producing carotenoids or their precursors.

20. The method according to claim 3, wherein the vector employed in the transformation includes genetic information for producing carotenes or xanthophylls.

21. The method according to claim 3, wherein the vector employed in the transformation includes genetic information for producing astaxanthin, zeaxanthin, echinenone, β-cryptoxanthin, andonixanthin, adonirubin, canthaxanthin, 3-hydroxyechinenone, 3′-hydroxyechinenone, lycopene, β-carotene, α-carotene, lutein, phytofluene, bixin or phytoene.

22. A method for providing at least one highly pure carotenoid and a foodstuff comprising carotenoid-producing organisms and at least the one carotenoid, which method comprises, after cultivation of carotenoid-producing genetically modified organisms of the Blakeslea genus according to claim 1, the following steps:
I) removal of the biomass,
IA) optional washing of the biomass with a solvent in which carotenoids are not soluble, in particular water,
IB) sterilization and cell disruption of the biomass,
IC) optional drying and/or homogeneous distribution, and
II) partial extraction of the carotenoids from the disrupted biomass by means of a carotenoid-dissolving solvent and separation of said solvent from said biomass,
IIA)
1) removal of residual solvent from the carotenoid-containing biomass,
2) optional homogeneous suspension of the biomass, with a biomass solid content of >2% and <50%,
3) drying of the biomass or suspension for producing the foodstuff,
IIB)
1) crystallization of the carotenoids from the solvent used and isolation of the carotenoid crystals, in particular by filtration.

23. The method according to claim 22, wherein the at least one carotenoid is selected from the group consisting of carotenes and xanthophylls.

24. The method according to claim 22, wherein the at least one carotenoid is selected from the group consisting of astaxanthin, zeaxanthin, echinenone, β-cryptoxanthin, andonixanthin, adonirubin, canthaxanthin, 3-hydroxyechinenone, 3′-hydroxy-echinenone, lycopene, β-carotene, lutein, phytofluene, bixin and phytoene.

25. The method according to claim 22, wherein the at least one carotenoid is astaxanthin, zeaxanthin, bixin or phytoene.

26. The method according to claim 22, wherein sterilization and cell disruption are carried out using steam or microwave radiation.

27. The method according to claim 22, wherein the carotenoids are extracted from the biomass using dichloromethane or supercritical carbon dioxide or tetrahydrofuran.

28. The method according to claim 27, wherein the carotenoids dissolved in supercritical carbon dioxide are isolated directly or are taken up in dichloromethane.

29. The method according to claim 22, wherein the carotenoids are extracted from the biomass in a one-stage or, if appropriate, multistage process.

30. The method according to claim 22, wherein solvents are removed from the biomass in step IIA1) using steam distillation.

31. The method according to claim 22, wherein drying in step IIA3) is carried out using spray drying or contact drying.

32. The method according to claim 22, wherein crystallization in step IIB1) is carried out by replacing the solvent gradually with a solvent in which carotenoids are not soluble.

33. The method according to claim 32, wherein the solvent used is replaced with water or with a lower alcohol.

34. The method according to claim 13, wherein the genetically modified organism of the Blakeslea genus can be produced by transformation with a vector which comprises a sequence selected from the group consisting of SEQ ID NOs: 37-51 and SEQ ID NO: 62.

35. A method for producing a foodstuff comprising organisms of the Blakeslea genus and at least one carotenoid, which method comprises, after cultivation of carotenoid-producing genetically modified organisms of the Blakeslea genus according to claim 1, the following steps:
I) homogeneous suspension of the solids of the culture broth, and
IIA) for a biomass solid content of the culture broth of >2%:
1) optional concentration of the culture broth to give a solid content of <50%, and
2) drying of the culture broth to produce the foodstuff, or
IIB) for a solid content of <2% of the culture broth,
1) concentration of the culture broth to give a solid content of >2% and <50%, and
2) drying of the suspension to produce the foodstuff, or
IIC) independently of the solid content of the culture broth,
1) removal of the biomass,
2) optional washing of the biomass with solvents in which carotenoids are not soluble, in particular water,
3) sterilization and cell disruption,
4) optional drying and homogeneous distribution,
5) partial extraction of the carotenoids from the biomass using a carotenoid-dissolving solvent,
5a) removal of the carotenoid-containing biomass from the carotenoid-containing solvent,
5b) removal of residual solvent from the biomass, and
5c) drying of the biomass to produce the foodstuff,
6) crystallization of the carotenoids from the solvent used in 5a) and isolation of the carotenoid crystals, in particular by filtration.

36. The method according to claim 35, wherein the at least one carotenoid is selected from the group consisting of carotenes and xanthophylls.

37. The method according to claim 35, wherein the at least one carotenoid is selected from the group consisting of astaxanthin, zeaxanthin, echinenone, β-cryptoxanthin, andonixanthin, adonirubin, canthaxanthin, 3-hydroxyechinenone, 3′-hydroxyechinenone, lycopene, β-carotene, lutein, bixin and phytoene.

38. The method according to claim 35, wherein the at least one carotenoid is astaxanthin, zeaxanthin, bixin or phytoene.

39. The method according to claim 35, wherein sterilization and cell disruption in step IIC3) are carried out using steam or microwave radiation.

40. The method according to claim 35, wherein the carotenoids are extracted from the biomass in step IIC5) using dichloromethane or supercritical carbon dioxide.

41. The method according to claim 40, wherein the carotenoids dissolved in supercritical carbon dioxide are isolated directly or are taken up in dichloromethane.

42. The method according to claim 35, wherein the carotenoids are extracted from the biomass in a one-stage or, if appropriate, multistage process.

43. The method according to claim 35, wherein solvents are removed from the biomass in step IIC5b) using steam distillation.

44. The method according to claim 35, wherein drying in any of steps IIA2), IIB2) and IIC5c) is carried out using spray drying or contact drying.

45. The method according to claim 35, wherein crystallization in step IIC6) is carried out by replacing the solvent gradually with a solvent in which carotenoids are not soluble.

46. The method according to claim 45, wherein the solvent used is replaced with water or with a lower alcohol.

47. The method according to claim 35, wherein the genetically modified organism of the Blakeslea genus can be produced by transformation with a vector which has a sequence selected from the group consisting of SEQ ID NOs: 37-51 and SEQ ID NO: 62.

48. A foodstuff, in particular animal feedstuff, which can be produced by the method of claim 1.

49. A food supplement, in particular animal feed supplement, which can be produced by the method of claim 1.

50. A method for producing the foodstuff and animal feedstuff of claim 48 comprising a fermentation.

51. A method for producing the food supplement and animal feed supplement of claim 49 comprising a fermentation.

52. The method according to claim 35, wherein at least two products of the group consisting of foodstuff, food supplement, animal feedstuff and animal feed supplement can be obtained from a fermentation.

53. The method according to claim 35, wherein the carotenoids are incorporated in the production of cosmetic, pharmaceutical, dermatological preparations, foodstuffs, food supplements, animal feedstuff or animal feed supplement.

54. The method according to claim 8, wherein the hygromycin resistance gene (hph) is from E. coli.

55. The method according to claim 33, wherein the solvent is replaced with methanol.

56. The method according to claim 46, wherein the solvent is replaced with methanol.
